SEQUENCE LISTING (45 sequences)

SEQ ID NO: 1 - DNA, 725 bp, Homo sapien
  CDS (27)..(584)
ggccaggccagctggacgggcacaccatgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagagcagagcctctagcctcccctggagctccggcctgcccagcaggtcagaagccagagccagcctgctggcctcagctccgggttgggctgagatggctgtgccccaactcccattcacccaccatggacccaataataaacctggccccacccc

SEQ ID NO: 2 - protein, 185 aa, Homo sapien
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 3 - protein, 170 aa, Homo sapien
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSEQSL

SEQ ID NO: 4 - DNA, 2460 bp, Homo sapien
  CDS (22)..(2082)
ggccagctggacgggcacaccatgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcagccttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattgaaaaaaaaaaaaaaaaa

SEQ ID NO: 5 - protein, 686 aa, Homo sapien
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 6 - protein, 671 aa, Homo sapien
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 7 - DNA, 4900 bp, Homo sapien
cctgtcctgcctgcctggaactctgagcaggctggagtcatggagtcgattcccagaatcccagagtcagggaggctgggggcaggggcaggtcactggacaaacagatcaaaggtgagaccagcgtaggactgcagaccaggccaggccagctggacgggcacaccatgaggtaggtgggcgccacagcctccctgcagggtgtggggtgggagcacaggcctgggcctcaccgcccctgccctgcccataggctgctgaccctcctgggccttctgtgtggctcggtggccacccccttaggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaaggtgccgtcagacgggagggctggggtttctcagggtcggggggtccccaaggagtagccagggttcagggacacctgggagcaggggccaggcttggccaggagggagatcaggcctgggtcttgccttcactccctgtgacacctgaccccacagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgagggtgagccaagaggggtcctgcaacatctcagtctgcgcagctggctgtgggggtaactctgtcttaggccaggcagccctgccttcagtttccccacctttcccagggcaggggagaggcctctggcctgacatcatccacaatgcaaagaccaaaacagccgtgacctccattcacatgggctgagtgccaactctgagccagggatctgaggacagcatcgcctcaagtgacgcagggactggccgggcgcggcagctcacgcctgtaattccagcactttgggaggccgaggctggcttgataatttgagggtcaggagttcaaggccagccagggcaacacggtgaaactctatctccactaaaactacaaaaattagctgggcgtggtggtgcgcacctggaatcccagctactagggaggctgaggcaggagaattgcttgaacctgcgaggtggaggctgcagtgaacagagattgcaccactacactccacctgggcgacagactagactccgtctcaaaaaacaaaaaacaaaaaccacgcagggccgagggcccatttacaagctgacaaagtgggccctgccagcgggagcgctgcaggatgtttgattttcagatcccagtccctgcagagaccaactgtgtgacctctggcaagtggctcaatttctctgctccttagaagctgctgcaagggttcagcgctgtagccccgccccctgggtttgattgactcccctcattagctgggtgacctcggccggacactgaaactcccactggtttaacagaggtgatgtttgcatctttctcccagcgctgctgggagcttgcagcgaccctaggcctgtaaggtgattggcccggcaccagtcccgcaccctagacaggacctaggcctcctctgaggtccactctgaggtcatggatctcctgggaggagtccaggctggatcccgcctctttccctcctgacggcctgcctggccctgcctctcccccagacattgacgagtgccaggtggccccgggagaggcgcccacctgcgaccaccactgccacaaccacctgggcggtttctactgctcctgccgcgcaggctacgtcctgcaccgtaacaagcgcacctgctcaggtgagggaggctgcctgggccccaacgcaccctctcctgggatacccggggctcctcagggccattgctgctctgcccaggggtgcggagggcctgggcctggacactgggtgcttctaggccctgctgcctccagctccccttctcagccctgcttcccctctcagcagccaggctcatcagtgccaccctgccctagcactgagactaattctaacatcccactgtgtacctggttccacctgggctctgggaacccctcatgtagccacgggagagtcggggtatctaccctcgttccttggactgggttcctgttccctgcactgggggacgggccagtgctctggggcgtgggcagccccaccctgtggcgctgaccctgctcccccgactcggtttctcctctcggggtctctccttgcctctctgatctctcttccagagcagagcctctagcctcccctggagctccggctgcccagcaggtcagaagccagagccaggctgctggcctcagctccgggttgggctgagatgctgtgccccaactcccattcacccaccatggacccaataataaacctggccccaccccacctgctgccgcgtgtctctggggtgggagggtcgggaggcggtggggcgcgctcctctctgcctaccctcctcacagcctcatgaaccccaggtctgtgggagcctcctccatggggccacacggtccttggcctcaccccctgttttgaagatggggcactgaggccggagaggggtaaggcctcgctcgagtccaggtccccagaggctgagcccagagtaatcttgaaccacccccattcagggtctggcctggaggagcctgacccacagaggagacaccctgggagatattcattgaggggtaatctggtcccccgcaaatccaggggtgattcccactgccccataggcacagccacgtggaagaaggcaggcaatgttggggctcctcacttcctagaggcctcacaactcaaatgccccccactgcagctgggggtggggtggtggtatgggatggggaccaagccttccttgaaggatagagcccagcccaacaccccgccccgtggcagcagcatcacgtgttccagcgaggaaggagagcaccagactcagtcatgatcactgttgccttgaacttccaagaacagccccagggcaagggtcaaaacaggggaaagggggtgatgagagatccttcttccggatgttcctccaggaaccagggggctggctggtcttggctgggttcgggtaggagacccatgatgaataaacttgggaatcactggggtggctgtaagggaatttaggggagctccgaaggggcccttaggctcgaggagatgctcctctcttttcccgaattcccagggacccaggagagtgtcccttcttcctcttcctgtgtgtccatccacccccgccccccgccctggcagagctggtggaactcagtgctctagcccctaccctggggttgcgactctggctcaggacaccaccacgctccctgggggtgtgagtgagggcctgtgcgctccatcccgagtgctgcctgtttcagctaaagcctcaaagcaagagaaaccccctctctaagcggcccctcagccatcgggtgggtcgtttggtttctgggtaggcctcaggggctggccacctgcagggcccagcccaacccagggatgcagatgtcccagccacatccctgtcccagtttcctgctccccaaggcatccaccctgctgttggtgcgagggctgatagagggcacgccaagtcactcccctgcccttccctccttccagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaaggtctggctcctgggcccctcatcttgtcccagatcctcccccttcagcccagctgcaccccctacttcctgcagcatggcccccaccacgttcccgtcaccctcggtgaccccacctcttcaggtgctctatggaggtcaaggctggggcttcgagtacaagtgtgggaggcagagtggggaggggcaccccaatccatggcctgggttggcctcattggctgtccctgaaatgctgaggaggtgggttacttccctccgcccaggccagacccaggcagctgctccccagctttcatgagcttctttctcagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagtgagcaagtgggctcagatccttggtggaagcgcagagctgcctctctctggagtgcaaggagctgtagagtgtagggctcttctgggcaggactaggaagggacaccaggtttagtggtgctgaggtctgaggcagcagcttctaaggggaagcacccgtgccctcctcagcagcacccagcatcttcaccactcattcttcaaccacccattcacccatcactcatcttttacccacccaccctttgccactcatccttctgtccctcatccttccaaccattcatcaatcacccacccatccatcctttgccacacaaccatccacccattcttctacctacccatcctatccatccatccttctatcagcatccttctaccacccatccttcgttcggtcatccatcatcatccatccatc

SEQ ID NO: 8 - protein, 136 aa, Homo sapien
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAA

SEQ ID NO: 9 - protein, 15 aa, Artificial Sequence
  Synthetic Peptide
TPLGPKWPEPVFGRL

SEQ ID NO: 10 - protein, 43 aa, Artificial Sequence
  Synthetic Peptide
TAPPGYRLRLYFTHFDLELSHLCEYDFVKLSSGAKVLATLCGQ

SEQ ID NO: 11 - protein, 35 aa, Artificial Sequence
  Synthetic Peptide
DTERAPGKDTFYSLGSSLDITFRSDYSNEKPFTGF

SEQ ID NO: 12 - protein, 5 aa, Artificial Sequence
  Synthetic Peptide
SEQSL

SEQ ID NO: 13 - protein, 37 aa, Artificial Sequence
  Synthetic Peptide
APTCGHHCHNHLGGFYCSCRAGYVLHRNKRTCSEQSL

SEQ ID NO: 14 - protein, 248 aa, Homo Sapien
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 15 - protein, 6 aa, Artificial Sequence
  Synthetic peptide consensus sequence
XGKXGP

SEQ ID NO: 16 - protein, 5 aa, Artificial Sequence
  Synthetic Peptide
XGKLG

SEQ ID NO: 17 - protein, 16 aa, Artificial Sequence
  Synthetic Peptide
GLRGLQGPXGKLGPXG

SEQ ID NO: 18 - protein, 27 aa, Artificial Sequence
  Synthetic Peptide
GPXGPXGLRGLQGPXGKLGPXGPXGPX

SEQ ID NO: 19 - protein, 53 aa, Artificial Sequence
  Synthetic Peptide
GKDGRDGTKGEKGEPGQGLRGLQGPXGKLGPXGNXGPSGSXGPKGQKGDXGKS

SEQ ID NO: 20 - protein, 33 aa, Artificial Sequence
  Synthetic Peptide
GAXGSXGEKGAXGPQGPXGPXGKMGPKGEXGDX

SEQ ID NO: 21 - protein, 45 aa, Artificial Sequence
  Synthetic Peptide
GCXGLXGAXGDKGEAGTNGKRGERGPXGPXGKAGPXGPNGAXGEX

SEQ ID NO: 22 - DNA, 408 bp, Homo Sapien
atgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagcc

SEQ ID NO: 23 - DNA, 24 bp, Artificial Sequence
  Oligonucleotide
atgaggctgctgaccctcctgggc

SEQ ID NO: 24 - DNA, 27 bp, Artificial Sequence
  Oligonucleotide
acccccttgggcccgaagtggcctgaa

SEQ ID NO: 25 - DNA, 33 bp, Artificial Sequence
  Oligonucleotide
gacattaccttccgctccgactccaacgagaag

SEQ ID NO: 26 - DNA, 15 bp, Artificial Sequence
  Oligonucleotide
gagcagagcctctag

SEQ ID NO: 27 - DNA, 15 bp, Artificial Sequence
  Oligonucleotide
ctagaggctctgctc

SEQ ID NO: 28 - DNA, 26 bp, Artificial Sequence
  Synthetic oligo
cgggatccatgaggctgctgaccctc

SEQ ID NO: 29 - DNA, 38 bp, Artificial Sequence
  Synthetic oligo
ggggtaccctagaggctctgctctgagcaggtgcgctt

SEQ ID NO: 30 - DNA, 25 bp, Artificial Sequence
  Synthetic oligo
tgcggccgctgtaggtgctgtcttt

SEQ ID NO: 31 - DNA, 23 bp, Artificial Sequence
  Synthetic oligo
ggaattcactcgttattctcgga

SEQ ID NO: 32 - DNA, 17 bp, Artificial Sequence
  Synthetic oligo
tccgagaataacgagtg

SEQ ID NO: 33 - DNA, 29 bp, Artificial Sequence
  Synthetic oligo
cattgaaagctttggggtagaagttgttc

SEQ ID NO: 34 - DNA, 27 bp, Artificial Sequence
  Synthetic oligo
cgcggccgcagctgctcagagtgtaga

SEQ ID NO: 35 - DNA, 28 bp, Artificial Sequence
  Synthetic oligo
cggtaagcttcactggctcagggaaata

SEQ ID NO: 36 - DNA, 37 bp, Artificial Sequence
  Synthetic oligo
aagaagcttgccgccaccatggattggctgtggaact

SEQ ID NO: 37 - DNA, 31 bp, Artificial Sequence
  Synthetic oligo
cgggatcctcaaactttcttgtccaccttgg

SEQ ID NO: 38 - DNA, 36 bp, Artificial Sequence
  Synthetic oligo
aagaaagcttgccgccaccatgttctcactagctct

SEQ ID NO: 39 - DNA, 26 bp, Artificial Sequence
  Synthetic oligo
cgggatccttctccctctaacactct

SEQ ID NO: 40 - protein, 9 aa, Artificial Sequence
  Synthetic Peptide
EPKSCDKTH

SEQ ID NO: 41 - DNA, 4960 bp, Homo Sapien
ccggacgtggtggcgcatgcctgtaatcccagctactcgggaggctgaggcaggagaattgctcgaaccccggaggcagaggtttggtggctcacacctgtaatcccagcactttgcgaggctgaggcaggtgcatcgctttggctcaggagttcaagaccagcctgggcaacacagggagacccccatctctacaaaaaacaaaaacaaatataaaggggataaaaaaaaaaaaaagacaagacatgaatccatgaggacagagtgtggaagaggaagcagcagcctcaaagttctggaagctggaagaacagataaacaggtgtgaaataactgcctggaaagcaacttctttttttttttttttttttttgaggtggagtctcactctgtcgtccaggctggagtgcagtggtgcgatctcggatcactgcaacctccgcctcccaggctcaagcaattctcctgcctcagcctcccgagtagctgggattataagtgcgcgctgccacacctggatgatttttgtatttttagtagagatgggatttcaccatgttggtcaggctggtctcaaactcccaacctcgtgatccacccaccttggcctcccaaagtgctgggattacaggtataagccaccgagcccagccaaaagcgacttctaagcctgcaagggaatcgggaattggtggcaccaggtccttctgacagggtttaagaaattagccagcctgaggctgggcacggtggctcacacctgtaatcccagcactttgggaggctaaggcaggtggatcacctgagggcaggagttcaagaccagcctgaccaacatggagaaaccccatccctaccaaaaataaaaaattagccaggtgtggtggtgctcgcctgtaatcccagctacttgggaggctgaggtgggaggattgcttgaacacaggaagtagaggctgcagtgagctatgattgcagcactgcactgaagccggggcaacagaacaagatccaaaaaaaagggaggggtgaggggcagagccaggatttgtttccaggctgttgttacctaggtccgactcctggctcccagagcagcctgtcctgcctgcctggaactctgagcaggctggagtcatggagtcgattcccagaatcccagagtcagggaggctgggggcaggggcaggtcactggacaaacagatcaaaggtgagaccagcgtagggctgcagaccaggccaggccagctggacgggcacaccatgaggtaggtgggcgcccacagcctccctgcagggtgtggggtgggagcacaggcctgggccctcaccgcccctgccctgcccataggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaaggtgccgtcaggacgggagggctggggtttctcagggtcggggggtccccaaggagtagccagggttcagggacacctgggagcaggggccaggcttggccaggagggagatcaggcctgggtcttgccttcactccctgtgacacctgaccccacagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgagggtgagccaagaggggtcctgcaacatctcagtctgcgcagctggctgtgggggtaactctgtcttaggccaggcagccctgccttcagtttccccacctttcccagggcaggggagaggcctctggcctgacatcatccacaatgcaaagaccaaaacagccgtgacctccattcacatgggctgagtgccaactctgagccagggatctgaggacagcatcgcctcaagtgacgcagggactggccgggcgcagcagctcacgcctgtaattccagcactttgggaggccgaggctggctgatcatttgaggtcaggagttcaaggccagccagggcaacacggtgaaactctatctccactaaaactacaaaaattagctgggcgtggtggtgcgcacctggaatcccagctactagggaggctgaggcaggagaattgcttgaacctgcgaggtggaggctgcagtgaacagagattgcaccactacactccagcctgggcgacagagctagactccgtctcaaaaaacaaaaaacaaaaacgacgcaggggccgagggccccatttacagctgacaaagtggggccctgccagcgggagcgctgccaggatgtttgatttcagatcccagtccctgcagagaccaactgtgtgacctctggcaagtggctcaatttctctgctccttaggaagctgctgcaagggttcagcgctgtagccccgccccctgggtttgattgactcccctcattagctgggtgacctcgggccggacactgaaactcccactggtttaacagaggtgatgtttgcatctttctcccagcgctgctgggagcttgcagcgaccctaggcctgtaaggtgattggcccggcaccagtcccgcaccctagacaggacgaggcctcctctgaggtccactctgaggtcatggatctcctgggaggagtccaggctggatcccgcctctttccctcctgacggcctgcctggccctgcctctcccccagacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcacgcttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattgaaaaaaaaaaaaaaaaa

SEQ ID NO: 42 - DNA, 3236 bp, Homo Sapien
ccggacgtggtggcgcatgcctgtaatcccagctactcgggaggctgaggcaggagaattgctcgaaccccggaggcagaggtttggtggctcacacctgtaatcccagcactttgcgaggctgaggcaggtgcatcgctttggctcaggagttcaagaccagcctgggcaacacagggagacccccatctctacaaaaaacaaaaacaaatataaaggggataaaaaaaaaaaaaagacaagacatgaatccatgaggacagagtgtggaagaggaagcagcagcctcaaagttctggaagctggaagaacagataaacaggtgtgaaataactgcctggaaagcaacttctttttttttttttttttttttgaggtggagtctcactctgtcgtccaggctggagtgcagtggtgcgatctcggatcactgcaacctccgcctcccaggctcaagcaattctcctgcctcagcctcccgagtagctgggattataagtgcgcgctgccacacctggatgatttttgtatttttagtagagatgggatttcaccatgttggtcaggctggtctcaaactcccaacctcgtgatccacccaccttggcctcccaaagtgctgggattacaggtataagccaccgagcccagccaaaagcgacttctaagcctgcaagggaatcgggaattggtggcaccaggtccttctgacagggtttaagaaattagccagcctgaggctgggcacggtggctcacacctgtaatcccagcactttgggaggctaaggcaggtggatcacctgagggcaggagttcaagaccagcctgaccaacatggagaaaccccatccctaccaaaaataaaaaattagccaggtgtggtggtgctcgcctgtaatcccagctacttgggaggctgaggtgggaggattgcttgaacacaggaagtagaggctgcagtgagctatgattgcagcactgcactgaagccggggcaacagaacaagatccaaaaaaaagggaggggtgaggggcagagccaggatttgtttccaggctgttgttacctaggtccgactcctggctcccagagcagcctgtcctgcctgcctggaactctgagcaggctggagtcatggagtcgattcccagaatcccagagtcagggaggctgggggcaggggcaggtcactggacaaacagatcaaaggtgagaccagcgtagggctgcagaccaggccaggccagctggacgggcacaccatgaggtaggtgggcgcccacagcctccctgcagggtgtggggtgggagcacaggcctgggccctcaccgcccctgccctgcccataggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaaggtgccgtcaggacgggagggctggggtttctcagggtcggggggtccccaaggagtagccagggttcagggacacctgggagcaggggccaggcttggccaggagggagatcaggcctgggtcttgccttcactccctgtgacacctgaccccacagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgagggtgagccaagaggggtcctgcaacatctcagtctgcgcagctggctgtgggggtaactctgtcttaggccaggcagccctgccttcagtttccccacctttcccagggcaggggagaggcctctggcctgacatcatccacaatgcaaagaccaaaacagccgtgacctccattcacatgggctgagtgccaactctgagccagggatctgaggacagcatcgcctcaagtgacgcagggactggccgggcgcagcagctcacgcctgtaattccagcactttgggaggccgaggctggctgatcatttgaggtcaggagttcaaggccagccagggcaacacggtgaaactctatctccactaaaactacaaaaattagctgggcgtggtggtgcgcacctggaatcccagctactagggaggctgaggcaggagaattgcttgaacctgcgaggtggaggctgcagtgaacagagattgcaccactacactccagcctgggcgacagagctagactccgtctcaaaaaacaaaaaacaaaaacgacgcaggggccgagggccccatttacagctgacaaagtggggccctgccagcgggagcgctgccaggatgtttgatttcagatcccagtccctgcagagaccaactgtgtgacctctggcaagtggctcaatttctctgctccttaggaagctgctgcaagggttcagcgctgtagccccgccccctgggtttgattgactcccctcattagctgggtgacctcgggccggacactgaaactcccactggtttaacagaggtgatgtttgcatctttctcccagcgctgctgggagcttgcagcgaccctaggcctgtaaggtgattggcccggcaccagtcccgcaccctagacaggacgaggcctcctctgaggtccactctgaggtcatggatctcctgggaggagtccaggctggatcccgcctctttccctcctgacggcctgcctggccctgcctctcccccagacattgacgagtgccaggtggccccgggagaggcgcccacctgcgaccaccactgccacaaccacctgggcggtttctactgctcctgccgcgcaggctacgtcctgcaccgtaacaagcgcacctgctcagagcagagcctctagcctcccctggagctccggctgcccagcaggtcagaagccagagccagcctgctggcctcagctccgggttgggctgagatgctgtgccccaactcccattcacccaccatggacccaataataaacctggccccaccccaaaaaaaaaaaaaaaaaa

SEQ ID NO: 43 - protein, 699 aa, Homo Sapien
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIQDHPEVPCPYDYIKIKVGPKVLGPFCGEKAPEPISTQSHSVLILFHSDNSAENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPKDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 44 - protein, 367 aa, Mus Musculus
MSLPCPQLLIFLGLLWSLVATLLGSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSALCSGQVFTGKSGYLSSPEYPQPYPKLSSCTYSIRLEDGFSVILDFVESFDVETHPEAQCPYDSLKIQTDKGEHGPFCGKTLPPRIETDSHKVTITFATDESGNHTGWKIHYTSTARPCPDPTAPPNGSISPVQAIYVLKDRFYVFCKTGFELLQGSVPLKSFTAVCQKDGSWDRPMPECS

SEQ ID NO: 45 - protein, 185 aa, Mus Musculus
MRLLIFLGLLWSLVATLLGSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSEQSL

(19) United States
(12) Patent Application Publication
(10) Pub. No.: US 2006/0018896 A1
(43) Pub. Date: Jan. 26, 2006

(54) Methods for treating conditions associated with lectin-dependent complement activation

(75) Inventors: Hans-Wilhelm Schwaeble, Mountsorrel (GB); Cordula Margaret Stover, Aylestone (GB); Clark E. Tedford, Poulsbo, WA (US); James B. Parent, Bainbridge Island, WA (US); Teizo Fujita, Fukushima (JP)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: University of Leicester, Leicester (GB); Omeros Corporation, Seattle, WA (US)

(21) Appl. No.: 11/150,887

(22) Filed: Jun. 9, 2005

Related U.S. Application Data
(60) Provisional application No. 60/579,034, filed on Jun. 10, 2004.

(51) Int. Cl. A61K 39/395; A61K 38/17

(52) U.S. Cl. 424/130.1; 514/12

(57) ABSTRACT

In one aspect, the invention provides methods of inhibiting the effects of lectin-dependent complement activation in a living subject. The methods comprise the step of administering, to a subject in need thereof, an amount of a MAp19 inhibitory agent effective to inhibit lectin-dependent complement activation. In some embodiments, the MAp19 inhibitory agent inhibits cellular injury associated with lectin-mediated complement pathway activation, while leaving the classical (C1q-dependent) pathway component of the immune system intact. In another aspect, the invention provides MAp19 specific antibodies that do not bind to MASP-2 and methods of producing MAp19 specific antibodies. In another aspect, the invention provides compositions for inhibiting the effects of lectin-dependent complement activation, comprising a therapeutically effective amount of a MAp19 inhibitory agent and a pharmaceutically acceptable carrier.

### CROSS-REFERENCE(S) TO RELATED APPLICATION(S)

This application claims the benefit of U.S. Provisional Application No. 60/579,034, filed Jun. 10, 2004.

### FIELD OF THE INVENTION

The present invention relates to methods of treating conditions associated with lectin-dependent complement activation.

### BACKGROUND OF THE INVENTION

The complement system has been implicated as contributing to the pathogenesis of numerous acute and chronic disease states, including: myocardial infarction, revascularization following stroke, ARDS, reperfusion injury, septic shock, capillary leakage following thermal burns, postcardiopulmonary bypass inflammation, transplant rejection, rheumatoid arthritis, multiple sclerosis, myasthenia gravis, and Alzheimer's disease. In almost all of these conditions, complement is not the cause, but is one of several factors involved in pathogenesis. Nevertheless, complement activation may be a major pathological mechanism and represents an effective point for clinical control in many of these disease states. The growing recognition of the importance of complement-mediated tissue injury in a variety of disease states underscores the need for effective complement inhibitory drugs. No approved drugs that inhibit complement damage currently exist.

Studies performed over the past two decades have led to the discovery of a novel route of complement activation termed the lectin pathway, which is increasingly recognized as an important component of innate complement immune defense system. This pathway is triggered by oligomeric lectins (mannan-binding lectin (MBL), H-ficolin, M-ficolin, and L-ficolin) that recognize arrays of neutral carbohydrates on the surface of pathogens and damaged cells and share the ability to associate with enzymes called mannan-binding lectin-associated serine proteases (MASPs) (Fujita, T., Nature Rev. Immunol. 2:346-353, 2002; Holnskov, U., et al., Ann. Rev. Immunol. 21:547-578, 2003; Teh et al., Immunology, 101: 225-232 (2000)).

Ikeda et al. first demonstrated that MBL could activate the complement system upon binding to yeast mannan-coated erythrocytes in a complement component C4-dependent manner (Ikeda, K., et al., J. Biol. Chem. 262:7451-7454, 1987). MBL, a member of the collectin protein family, is a calcium-dependent lectin that binds carbohydrates with 3- and 4-hydroxy groups oriented in the equatorial plane of the pyranose ring. MBL recognizes the carbohydrate patterns that commonly decorate microorganisms such as bacteria, yeast, parasites and certain viruses. Prominent ligands with high affinity for MBL are thus D-mannose and N-acetyl D-glucosamine, while carbohydrates not fitting this steric requirement have low or undetectable affinity for MBL (Weis, W. I., et al., Nature 360:127-134, 1992). The interaction between MBL and monovalent sugars is also extremely weak, with dissociation constants typically in the 2 mM range. However, MBL achieves tight, specific binding to glycan ligands by interaction with multiple monosaccharide residues simultaneously (Lee, R. T., et al., Archiv. Biochem. Biophys. 299:129-136, 1992).

MBL does not recognize D-galactose and sialic acid, the penultimate and ultimate sugars that usually decorate “mature” complex glycoconjugates present on mammalian plasma and cell surface glycoproteins. This binding specificity for non-mammalian cell surface glycoconjugates is thought to help protect from self activation. However, MBL does bind with high affinity to clusters of high-mannose “precursor” glycans on N-linked glycoproteins and glycolipids sequestered in the endoplasmic reticulum and Golgi of mammalian cells (Maynard, Y., et al., J. Biol. Chem. 257:3788-3794, 1982). Therefore, damaged cells are potential targets for lectin pathway activation via MBL binding.

The ficolins possess a different type of lectin domain than MBL, called the fibrinogen-like domain (Matsushita, M., et al., Immunobiology 205:490, 2002). Ficolins bind sugar residues in a Ca⁺⁺-independent manner. In humans, three kinds of ficolins, L-ficolin, M-ficolin and H-ficolin, have been identified. Both serum ficolins, L-ficolin and H-ficolin, have in common a specificity for N-acetyl-D-glucosamine; however, H-ficolin also binds N-acetyl-D-galactosamine. The difference in sugar specificity of L-ficolin, H-ficolin and MBL means that the different lectins may be complementary and target different, although overlapping, glycoconjugates. Consistent with this concept is the recent report that, of the known lectins in the lectin pathway, only L-ficolin binds specifically to lipoteichoic acid, a cell wall glycoconjugate found on all Gram-positive bacteria (Lynch, N. J., et al., J. Immunol. 172:1198-1202, 2004). The collectins (i.e., MBL) and the ficolins bear no significant similarity in amino acid sequence. However, the two groups of proteins have similar domain organizations and assemble into oligomeric structures, which maximize the possibility of multisite binding. In serum, MBL, H-ficolin and L-ficolin are present as oligomers of homotrimeric subunits, each comprising N-terminal collagen-like fibers prolonged by carbohydrate recognition domains.

The serum concentrations of MBL are highly variable in healthy populations and this is genetically controlled by the polymorphism/mutations in both the promoter and coding regions of the MBL gene. As an acute phase protein, the expression of MBL is further upregulated during inflammation. L-ficolin is present in serum at similar concentrations as MBL. Therefore, the L-ficolin arm of the lectin pathway is potentially comparable to the MBL arm in strength. MBL and ficolins can also function as opsonins, which require interaction of these proteins with phagocyte receptors (Kuhlman, M. et al., J. Exp. Med. 169:1733, 1989; Matsushita, M., et al., J. Biol. Chem. 271:2448-54, 1996). However, the identities of the receptor(s) on phagocytic cells have not been established.

Human MBL forms a specific and high affinity interaction through its collagen-like domain with unique C1r/C1s-like serine proteases, termed MBL-associated serine proteases (MASPs). To date, three MASPs have been described. First a single enzyme “MASP” was identified and characterized as the enzyme responsible for the initiation of the complement cascade (i.e., cleaving complement components C2 and C4) (Ji, Y. H., et al., J. Immunol. 150:571-578, 1993). Later, it turned out that MASP is in fact a mixture of two proteases: MASP-1 and MASP-2 (Thiel, S., et al., Nature 386:506-510, 1997). However, it was demonstrated that the MBL-MASP-2 complex alone is sufficient for complement activation (Vorup-Jensen, T., et al., J. Immunol. 165:2093-2100, 2000). Furthermore, only MASP-2 cleaved C2 and C4 at high rates (Ambrus, G., et al., J. Immunol. 170:1374-1382, 2003). Therefore, MASP-2 is the protease responsible for activating C4 and C2 to generate the C3 convertase, C4b2b. Recently, a third novel protease, MASP-3, has been isolated (Dahl, M. R., et al., Immunity 15:127-35, 2001). MASP-1 and MASP-3 are alternatively spliced products of the same gene. The MASP 1 and MASP 2 genes are located on chromosomes 3 and 1, respectively (Schwaeble, W., et al., Immunobiology 205:455-466, 2002). The biological functions of MASP-1 and MASP-3 remain to be resolved.

MASPs share identical domain organizations with those of C1r and C1s, the enzymatic components of the C1 complex in the classical complement pathway (Sim, R. B., et al., Biochem. Soc. Trans. 28:545, 2000). These domains include an N-terminal C1r/C1s/sea urchin Uegf/bone morphogenic protein (CUB) domain, an epidermal growth factor (EGF)-like domain, a second CUB domain, two complement control protein domains (CCP), and a chymotrypsin-like serine protease domain. As in the C1 proteases, activation of MASP-2 occurs through cleavage of an Arg-Ile bond adjacent to the serine protease domain, which splits the enzyme into disulfide-linked A and B chains, the latter consisting of the serine protease domain. Studies using recombinant human (Thielens, N. M., et al., J. Immunol. 166:5068-5077, 2001; Cseh, S., et al., J. Immunol. 169:5735-5743, 2002) and rat (Chen, C. B., et al., J. Biol. Chem. 276:25894-902, 2001) proteins have shown that MASP-1, MASP-2 and MASP-3 each associate as homodimers through their N-terminal CUB1-EGF moieties. Likewise, the MASPs each individually form Ca⁺⁺-dependent complexes with MBL and L-ficolin. The binding involves primarily the CUB1-EGF domains of each protein, but is strengthened by the CUB2 domain, which decreases the dissociation rate constant (k_{off}) of the interaction (Cseh, S., et al., J. Immunol. 169:5735-5743, 2002; Wallis, R., et al., J. Biol. Chem. 275:30962-69, 2000). All MASP-2 in serum is present in complexes with MBL and ficolins (Moller-Kristensen, M., et al., J. Immunol. Methods 282:159-67, 2003). MBL associates with all MASPs through similar sites located in the same area of collagen-like domain on MBL (Wallis, R., et al., J. Biol. Chem. 279:14065-73, 2004). Recently, a genetically determined deficiency of MASP-2 was described (Stengaard-Pedersen, K., et al., New Eng. J. Med. 349:554-560, 2003). The mutation of a single nucleotide leads to an Asp-Gly exchange in the CUB1 domain and renders MASP-2 incapable of binding to MBL.

MBL and the ficolins are also associated with a nonenzymatic protein referred to as MBL-associated protein of 19 kDa (MAp19) (Stover, C. M., J. Immunol. 162:3481-90, 1999) or small MBL-associated protein (sMAP) (Takahashi, M., et al., Int. Immunol. 11:859-863, 1999). MAp19 is formed by alternative splicing of the MASP 2 gene product and comprises the N-terminal CUB1-EGF domains of MASP-2 prolonged by four unique residues at its C-terminal end (Glu, Gln, Ser and Leu). MAp19 binds to MBL with lower affinity than the full size MASP-2 because it lacks the second CUB domain that is directly involved in the interaction (Thielens, N. M., et al., J. Immunol. 166, 5068-77, 2001; Chen, C. B., et al., J. Biol. Chem. 276:25894-902, 2001).

The structure of human MAp19 has recently been solved by X-ray crystallography (Gregory, L. A., et al., J. Biol. Chem., 279(28):29391-7, 2004). It shows a head to tail homodimer held together by interactions between the CUB1 domain of one monomer and the EGF domain of the counterpart. A Ca⁺⁺ ion bound to each EGF domain stabilizes the dimer interfaces. A second Ca⁺⁺ ion is bound to the distal end of each CUB1 domain, through six ligands contributed by Glu52, Asp60, Asp105, Ser107 Asn108 and a water molecule. Site-directed mutagenesis studies were used to identify the residues on MAp19 involved in the interaction with MBL and L-ficolin. Mutations at Tyr59, Asp60, Glu83, Asp105, Tyr106 and Glu109 either strongly decreased or abolished interaction with both MBL and L-ficolin. These mutations map a common binding site for these proteins located at the distal end of each CUB1 module and stabilized by the Ca⁺⁺ ion. Consistent with these results, a naturally occurring mutation Asp105Gly has been described recently in human MASP-2 and shown to result in a complete loss of interaction with MBL (Stengaard-Pedersen, K., et al., 2003). Not surprisingly, the structure of human MAp19 determined by Gregory, L. A., et al., 2004, appears to resemble in many respects the structure of the homologous regions in the CUB1-EGF-CUB2 segment of rat MASP-2 that was previously solved by X-ray crystallography (Feinberg, H., et al., EMBO J. 22:2348-2359, 2003). The rat and mouse homologues of human MASP-2 and MAp19 have been studied and are surprisingly similar to the human forms at both the genomic and primary structure levels (Stover, C. M., et al., J. Immunol. 163:6848-6859, 1999).

The lectin pathway is widely thought to have a major role in host defense against infection. Strong evidence for the involvement of MBL in host defense comes from analysis of patients with decreased serum levels of functional MBL (Kilpatrick, D. C., Biochim. Biophys. Acta 1572:401-413, 2002). Such patients display susceptibility to recurrent bacterial and fungal infections. These symptoms are usually evident early in life, during an apparent window of vulnerability as maternally derived antibody titer wanes, but before a full repertoire of antibody responses develops. This syndrome often results from mutations at several sites in the collagenous portion of MBL, which interfere with proper formation of MBL oligomers. However, since MBL can function as an opsonin independent of complement, it is not known to what extent the increased susceptibility to infection is due to impaired complement activation.

The role of the lectin pathway in the pathogenesis of non-infectious human diseases is just beginning to be evaluated. Recent studies provide evidence that activation of the lectin pathway can be responsible for complement activation and related inflammation in ischemia/reperfusion injury. Collard et al. (2001) reported that cultured endothelial cells subjected to oxidative stress bind MBL and show deposition of C3 upon exposure to human serum (Collard, C. D., et al., Am. J. Pathol. 156:1549-1556, 2000). Other studies have implicated the classical and alternative complement pathways in the pathogenesis of ischemia/reperfusion injury and the role of the lectin complement pathway in this disease remains controversial (Riedermann, N. C., et al., 162:363-367, 2003).

### SUMMARY OF THE INVENTION

In one aspect, the present invention provides a method of inhibiting the effects of lectin-dependent complement activation in a living subject. The method includes the step of administering, to a subject in need thereof, an amount of a MAp19 inhibitory agent effective to inhibit lectin-dependent complement activation. In another aspect of the invention, the MAp19 inhibitory agent inhibits complement activation via the lectin-dependent system without substantially inhibiting complement activation via the classical or C1q-dependent system, such that the C1q-dependent system remains functional. In yet another aspect, the MAp19 inhibitory agent inhibits cellular injury associated with lectin-mediated alternative complement pathway activation, while leaving the classical (C1q-dependent) pathway component of the immune system intact.

In some embodiments of these aspects of the invention, the MAp19 inhibitory agent is an anti-MAp19 antibody or fragment thereof. In further embodiments, the anti-MAp19 antibody has reduced effector function. In some embodiments, the MAp19 inhibitory agent is a MAp19 inhibitory peptide or a non-peptide MAp19 inhibitor. In another aspect, the present invention provides a MAp19 specific agent that does not bind to MASP-2. In some embodiments the MAp19 specific agent is an anti-MAp19 antibody or fragment(s) thereof. In some embodiments the MAp19 specific agent is a MAp19 specific inhibitory agent. In further embodiments, the anti-MAp19 specific antibody has reduced effector function. In some embodiments, the MAp19 specific inhibitory agent is a MAp19 specific inhibitory peptide or a non-peptide MAp19 specific inhibitor.

In another aspect, the present invention provides methods of producing murine anti-human MAp19 specific antibodies that do not bind to human MASP-2. The method comprises the steps of generating a MASP-2−/− transgenic animal, integrating a human MASP-2 transgene into said animal, introducing a human MAp19 derived antigen into said animal and selecting antibodies that specifically bind to human MAp19 that do not bind to human MASP-2. In one embodiment, the MASP-2−/− transgenic animal is also MAp19−/−. In some embodiments, the invention provides MAp19 specific antibodies produced by the methods of the invention. In some embodiments, the MAp19 specific antibodies produced by the method of the invention are MAp19 inhibitory agents. The MAp19 specific antibodies are useful for specifically detecting MAp19 in vivo and may be used, for example, in the diagnosis of MAp19 associated diseases and conditions. The MAp19 specific antibodies that are MAp19 inhibitory agents are useful in the methods of the invention directed to inhibiting MAp19-dependent complement activation.

In another aspect, the present invention provides compositions for inhibiting the effects of lectin-dependent complement activation, comprising a therapeutically effective amount of a MAp19 inhibitory agent and a pharmaceutically acceptable carrier. Methods are also provided for manufacturing medicaments for use in inhibiting the adverse effects of lectin-dependent complement activation for the treatment of each of the conditions, diseases and disorders described hereinbelow.

The methods, compositions and medicaments of the invention are useful for inhibiting the adverse effects of lectin-dependent complement activation in vivo in mammalian subjects, including humans suffering from an acute or chronic pathological condition or injury, as further described herein. Such conditions and injuries include, without limitation, lectin mediated complement activation in associated autoimmune disorders or inflammatory conditions.

In one aspect of the invention, methods are provided for inhibiting lectin-dependent complement activation in a subject experiencing ischemic reperfusion, including without limitation, after aortic aneurysm repair, cardiopulmonary bypass, vascular reanastomosis in connection with organ transplants (e.g., heart, lung, liver, kidney) and/or extremity/digit replantation, stroke, myocardial infarction, hemodynamic resuscitation following shock and/or surgical procedures, with a therapeutically effective amount of a MAp19 inhibitory agent in a pharmaceutical carrier.

In one aspect of the invention, methods are provided for inhibiting lectin-dependent complement activation in a subject suffering from or prone to atherosclerosis by treating the subject with a therapeutically effective amount of a MAp19 inhibitory agent in a pharmaceutical carrier.

In one aspect of the invention, methods are provided for inhibiting lectin-dependent complement activation in a subject experiencing a vascular condition, including without limitation, cardiovascular conditions, cerebrovascular conditions, peripheral (e.g., musculoskeletal) vascular conditions, renovascular conditions, mesenteric/enteric vascular, and revascularization to transplants and/or replants, by treating such patient with a therapeutically effective amount of a MAp19 inhibitory agent. Such conditions include without limitation the treatment of: vasculitis, including Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); venous gas embolus (VGE); and/or restenosis following stent placement, rotational atherectomy and/or percutaneous transluminal coronary angioplasty (PTCA).

In another aspect of the invention, methods are provided for inhibiting lectin-dependent complement activation in a subject suffering from inflammatory gastrointestinal disorders, including but not limited to: pancreatitis, diverticulitis and bowel disorders including Crohn's disease, ulcerative colitis, and irritable bowel syndrome.

In another aspect of the invention, methods are provided for inhibiting lectin-dependent complement activation in a subject suffering from pulmonary conditions, including but not limited to: acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, and emphysema.

In another aspect of the invention, methods are provided for inhibiting lectin-dependent complement activation in a subject that has undergone, is undergoing, or will undergo an extracorporeal reperfusion procedure, including but not limited to: hemodialysis, plasmapheresis, leukopheresis, extracorporeal membrane oxygenation (ECMO), heparin-induced extracorporeal membrane oxygenation LDL precipitation (HELP), and cardiopulmonary bypass (CPB).

In another aspect of the invention, methods are provided for inhibiting lectin-dependent complement activation in a subject suffering from a musculoskeletal condition, including but not limited to: osteoarthritis, rheumatoid arthritis, gout, neuropathic arthropathy, ochronosis, juvenile rheumatoid arthritis, psoriatic arthritis, ankylosing spondylitis or other spondyloarthropathies and crystalline arthropathies, or systemic lupus erythematosus (SLE).

In another aspect of the invention, methods are provided for inhibiting lectin-dependent complement activation in a subject suffering from a renal condition, including but not limited to: mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis, or IgA nephropathy.

In another aspect of the invention, methods are provided for inhibiting lectin-dependent complement activation in a subject suffering from a skin condition, including but not limited to: psoriasis, autoimmune bullous dermatoses, eosinophilic spongiosis, bullous pemphigoid, epidermolysis bullosa acquisita and herpes gestationis and other skin disorders, or from a thermal or chemical burn injury involving capillary leakage.

In another aspect of the invention, methods are provided for inhibiting lectin-dependent complement activation in a subject that has received an organ or other tissue transplant, including but not limited to: allotransplantation or xenotransplantation of whole organs (e.g., kidney, heart, liver, pancreas, lung, cornea, etc.) or grafts (e.g., valves, tendons, bone marrow, etc.).

In another aspect of the invention, methods are provided for inhibiting lectin-dependent complement activation in a subject suffering from a central nervous system disorder or injury or a peripheral nervous system disorder or injury, including but not limited to: multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, Parkinson's disease (PD), Alzheimer's disease (AD), Miller-Fisher syndrome, cerebral trauma and/or hemorrhage, demyelination, and meningitis.

In another aspect of the invention, methods are provided for inhibiting lectin-dependent complement activation in a subject suffering from a blood disorder, including but not limited to: sepsis or a condition resulting from sepsis including without limitation severe sepsis, septic shock, acute respiratory distress syndrome resulting from sepsis, and systemic inflammatory response syndrome. Related methods are provided for the treatment of other blood disorders, including hemorrhagic shock, hemolytic anemia, autoimmune thrombotic thrombocytopenic purpura (TTP), hemolytic uremic syndrome (HUS) or other marrow/blood destructive conditions.

In another aspect of the invention, methods are provided for inhibiting lectin-dependent complement activation in a subject suffering from a urogenital condition, including but not limited to: painful bladder disease, sensory bladder disease, chronic abacterial cystitis and interstitial cystitis, male and female infertility, placental dysfunction, miscarriage, and pre-eclampsia.

In another aspect of the invention, methods are provided for inhibiting lectin-dependent complement activation in a subject suffering from nonobese diabetes (Type-1 diabetes or Insulin dependent diabetes mellitus) or from angiopathy, neuropathy or retinopathy complications of Type-1 or Type-2 (adult onset) diabetes.

In another aspect of the invention, methods are provided for inhibiting lectin-dependent complement activation in a subject being treated with chemotherapeutics and/or radiation therapy, including without limitation, for the treatment of cancerous conditions, by administering a MAp19 inhibitor to such a patient perichemotherapeutically or periradiation therapy, i.e., before and/or during and/or after the administration of chemotherapeutic(s) and/or radiation therapy. Perichemotherapeutic administration of MAp19 inhibitors may be useful for reducing the side-effects of chemotherapeutic or radiation therapy. In a still further aspect of the invention, methods are provided for the treatment of malignancies by administering a MAp19 inhibitory agent in a pharmaceutically acceptable carrier to a patient suffering from a malignancy.

In another aspect of the invention, methods are provided for inhibiting lectin-dependent complement activation in a subject suffering from an endocrine disorder, by administering a therapeutically effective amount of a MAp19 inhibitory agent in a pharmaceutical carrier to such a subject. Conditions subject to treatment in accordance with the present invention include, by way of nonlimiting example, Hashimoto's thyroiditis, stress, anxiety and other potential hormonal disorders involving regulated release of prolactin, growth or insulin-like growth factor, and adrenocorticotropin from the pituitary.

In another aspect of the invention, methods are provided for inhibiting lectin-dependent complement activation in a subject suffering from age-related macular degeneration or other complement mediated ophthalmologic condition by administering a therapeutically effective amount of a MAp19 inhibitory agent in a pharmaceutical carrier to a subject suffering from such a condition.

In another aspect of the invention, MAp19 protein compositions, medicaments and methods for using the same are provided for the treatment of MAp19 deficiency disorders.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a flowchart illustrating the new discovery that the lectin complement pathway requires MASP-2 and may also require MAp19 for lectin-dependent complement activation;

FIG. 2 is a diagram illustrating the genomic structure of human MAp19 and MASP-2;

FIG. 3A is a schematic diagram illustrating the domain structure of human MASP-2 protein;

FIG. 3B is a schematic diagram illustrating the domain structure of human MAp19 protein;

FIG. 4 is a diagram illustrating the murine MAp19 knockout strategy;

FIG. 5 is a diagram illustrating the murine MASP-2 knockout strategy;

FIG. 6A is a diagram illustrating the human MAp19 minigene construct;

FIG. 6B is a diagram illustrating the human MASP-2 minigene construct;

FIG. 7 presents results demonstrating that MAp19 deficiency leads to the loss of lectin mediated C4 complement activation as measured by the lack of C4b deposition on Mannan;

FIG. 8A presents results demonstrating that MASP-2 deficiency leads to the loss of lectin mediated C4 complement activation as measured by lack of C4b deposition on Mannan;

FIG. 8B presents results demonstrating that MASP-2 deficiency leads to the loss of lectin mediated C4 complement activation as measured by lack of C4b deposition on Zymosan;

FIG. 8C presents results demonstrating the relative C4 activation levels of serum samples obtained from MASP-2+/+, MASP-2+/− and MASP-2−/− as measured by C4b deposition on Mannan and Zymosan;

FIG. 9A presents results demonstrating that MASP-2 deficiency leads to the loss of lectin mediated C3 complement activation as measured by the lack of C3b deposition on Mannan;

FIG. 9B presents results demonstrating that MASP-2 deficiency leads to the loss of lectin mediated C3 complement activation as measured by the lack of C3b deposition on Zymosan;

FIG. 10 presents results demonstrating that the addition of murine recombinant MASP-2 to MASP-2−/− serum samples recovers lectin-pathway mediated C4 activation in a protein concentration dependent manner, as measured by C4b deposition on Mannan;

FIG. 11 presents results demonstrating that the classical pathway is functional in the MASP-2−/− strain; and

FIG. 12 presents results demonstrating that the lectin-dependent complement activation system is activated in the ischemia/reperfusion phase following abdominal aortic aneurysm repair.

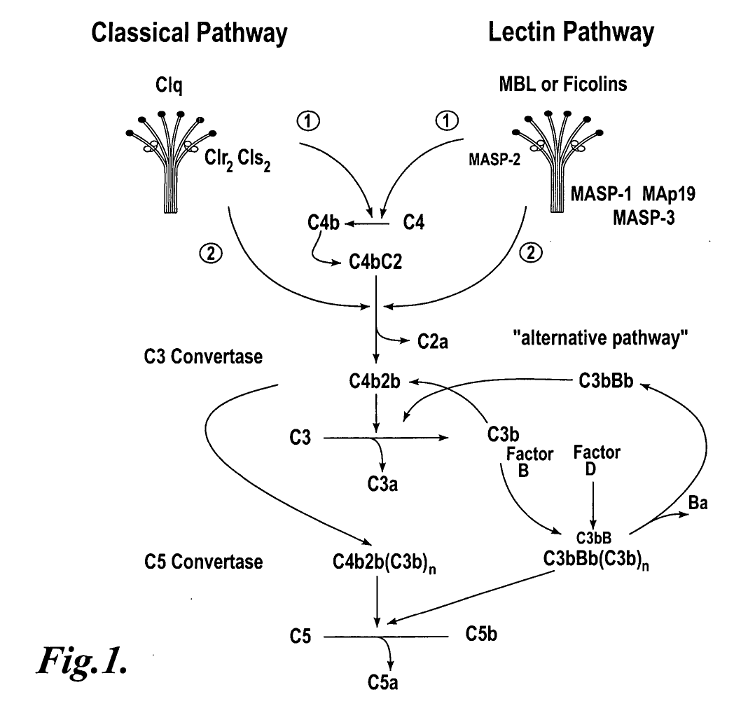

### DESCRIPTION OF THE SEQUENCE LISTING

SEQ ID NO:1 human MAp19 cDNA
SEQ ID NO:2 human MAp19 protein (with leader)
SEQ ID NO:3 human MAp19 protein (mature)
SEQ ID NO:4 human MASP-2 cDNA
SEQ ID NO:5 human MASP-2 protein (with leader)
SEQ ID NO:6 human MASP-2 protein (mature)
SEQ ID NO:7 human MASP-2 gDNA (exons 1-6)

MAp19 Derived Antigens:
SEQ ID NO:8 CUB1 sequence (aa 1-121)
SEQ ID NO:9 TPLGPKWPEPVFGRL (N-terminus of CUBI)
SEQ ID NO:10 TAPPGYRLRLYFTHFDLELSHLCEYDFVKLSSGAKVLATLCGQ (CUBI dimerization region)
SEQ ID NO:11 DTERAPGKDTFYSLGSSLDITFRSDYSNEKPFTGF (MBL binding region in CUBI domain)
SEQ ID NO:12 SEQSL (unique C-terminus)
SEQ ID NO:13 APTCGHHCHNHLGGFYCSCRAGYVLHRNKRTCSEQSL (unique C-terminal peptide)

Peptide Inhibitors:
SEQ ID NO:14 Human MBL protein
SEQ ID NO:15 OGK-X-GP consensus
SEQ ID NO:16 OGKLG Human MBL core binding
SEQ ID NO:17 GLR GLQ GPO GKL GPO G: Human MBL Triplet region demonstrating binding to MASP-2
SEQ ID NO:18 GPO GPO GLR GLQ GPO GKL GPO GPO GPO
SEQ ID NO:19 GKDGRDGTKGEKGEPGQGLRGLQGPOGKLGPOGNOGPSGSOGPKGQKGDOGK S
SEQ ID NO:20 GAOGSOGEKGAOGPQGPOGPOGKMGPKGEOGDO (human H-ficolin)
SEQ ID NO:21 GCOGLOGAOGDKGEAGTNGKRGERGPOGPOGKAGPOGPNGAOGEO (human L-ficolin)

Expression Inhibitors:
SEQ ID NO:22 ATG to CUBI domain (cDNA sense)
SEQ ID NO:23 5′ ATGAGGCTGCTGACCCTCCTGGGC 3′ Nucleotides 27-50 of SEQ ID NO:1 comprising MAp19 translation start site (sense)
SEQ ID NO:24 5′ACCCCCTTGGGCCCGAAGTGGCCTGAA3′ Nucleotides 72-98 of SEQ ID NO:1 comprising region encoding MAp19 N-terminal extension (sense)
SEQ ID NO:25 5′GACATTACCTTCCGCTCCGACTCCAACGAGAAG 3′ Nucleotides 366-401 of SEQ ID NO:1 comprising the region encoding MAp19 MBL binding site (sense)
SEQ ID NO:26 5′ GAGCAGAGCCTCTAG 3′ (Exon encoding EQSL sense)
SEQ ID NO:27 5′CTAGAGGCTCTGCTC 3′ (Exon encoding EQSL antisense)

Cloning Primers:
SEQ ID NO:28 cloning primer for recombinant expression
SEQ ID NO:29 cloning primer for recombinant expression
SEQ ID NO:30 to SEQ ID NO: 39 are humanized cloning primers
SEQ ID NO:40 is a 9 aa peptide bond
SEQ ID NO:41 is the MASP-2 minigene construct
SEQ ID NO:42 is the MAp19 minigene construct
SEQ ID NO: 43 is a human MASP-1 protein
SEQ ID NO: 44 is a murine MASP-2 protein
SEQ ID NO: 45 is a murine MAp19 protein

### DETAILED DESCRIPTION

The present invention is based upon the surprising discovery by the present inventors that MAp19 appears to be needed to initiate lectin mediated complement activation. Through the use of a knockout mouse model of MAp19−/−, which continues to express MASP-2 at a reduced level, the present inventors have shown that MAp19−/− mice are deficient in their ability to activate complement via serum carbohydrate recognition proteins (MBL, and the ficolins), thereby establishing MAp19 as a regulator of complement activation via the lectin-dependent pathway. The present invention also describes the use of MAp19 as a therapeutic target for inhibiting cellular injury associated with lectin-dependent complement activation while leaving the classical (C1q-dependent) pathway component of the immune system intact. In another aspect, the present invention provides MAp19 protein compositions, medicaments and methods for using the same for the treatment of disorders associated with MAp19 deficiency.

I. Definitions

Unless specifically defined herein, all terms used herein have the same meaning as would be understood by those of ordinary skill in the art of the present invention. The following definitions are provided in order to provide clarity with respect to the terms as they are used in the specification and claims to describe the present invention.

As used herein, the term “MASP-2-dependent complement activation” refers to alternative pathway complement activation that occurs via lectin-dependent MASP-2 activation.

As used herein, the term “alternative pathway” refers to complement activation that is triggered, for example, by zymosan from fungal and yeast cell walls, lipopolysaccharide (LPS) from Gram negative outer membranes, and rabbit erythrocytes, as well as from many pure polysaccharides, rabbit erythrocytes, viruses, bacteria, animal tumor cells, parasites and damaged cells, and which has traditionally been thought to arise from spontaneous proteolytic generation of C3b from complement factor C3.

As used herein, the term “lectin pathway” refers to complement activation that occurs via the specific binding of serum and non-serum carbohydrate-binding proteins including mannan-binding lectin (MBL) and the ficolins.

As used herein, the term “classical pathway” refers to complement activation that is triggered by an antibody bound to a foreign particle (i.e., an antigen) and requires binding of the recognition molecule C1q.

As used herein, the term “MAp19 inhibitory agent” refers to any agent that binds to or interacts with MAp19 and effectively inhibits lectin-dependent complement activation, including anti-MAp19 antibodies and MAp19 binding fragments thereof, natural and synthetic peptides, small molecules, soluble MAp19 receptors, expression inhibitors and isolated natural inhibitors. MAp19 inhibitory agents useful in the method of the invention may reduce lectin-dependent complement activation by greater than 20%, such as greater than 50%, such as greater than 90%. In one embodiment, the MAp19 inhibitory agents reduces lectin-dependent complement activation by greater than 90% (i.e., resulting in lectin-dependent complement activation of only 10% or less).

As used herein, the term “antibody” encompasses antibodies and fragments thereof derived from any antibody producing mammal (e.g., mouse, rat, rabbit, and primate, including human) that specifically bind to MAp19 polypeptides or portions thereof. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof.

As used herein, the term “antibody fragment” refers to a portion derived from or related to a full length anti-MAp19 antibody, generally including the antigen binding or variable region thereof. Illustrative examples of antibody fragments include Fab, Fab′, F(ab)₂, F(ab′)₂ and Fv fragments, scFv fragments, diabodies, linear antibodies, single-chain antibody molecules and multispecific antibodies formed from antibody fragments.

As used herein, a “single-chain Fv” or “scFv” antibody fragments comprises the V_{H} and V_{L} domains of an antibody, wherein these domains are present in a single polypeptide chain. Generally, the Fv polypeptide further comprises a polypeptide linker between the V_{H} and V_{L} domains, which enables the scFv to form the desired structure for antigen binding.

As used herein, a “chimeric antibody” is a recombinant protein that contains the variable domains and complementarity-determining regions derived from a non-human species (e.g., rodent) antibody, while the remainder of the antibody molecule is derived from a human antibody.

As used herein, a “humanized antibody” is a chimeric antibody which comprises a minimal sequence that conforms to specific complementarity-determining regions derived from non-human immunoglobulin that is transplanted into a human antibody framework. Humanized antibodies are typically recombinant proteins in which only the antibody complementarity-determining regions are of non-human origin.

As used herein, the term “mannan-binding lectin” (“MBL”), is equivalent to mannan-binding protein (“MBP”).

As used herein, the term “MAp19” is equivalent to small mannan-binding lectin (MBL)-associated protein “sMAP.”

As used herein, the “membrane attack complex” (“MAC”) refers to a complex of the terminal five complement components (C5-C9) that inserts into and disrupts membranes. Also referred to as C5b-9.

As used herein, “a subject” includes all mammals, including without limitation humans, non-human primates, dogs, cats, horses, sheep, goats, cows, rabbits, pigs and rodents.

As used herein, the amino acid residues are abbreviated as follows: alanine (Ala;A), asparagine (Asn;N), aspartic acid (Asp;D), arginine (Arg;R), cysteine (Cys;C), glutamic acid (Glu;E), glutamine (Gln;Q), glycine (Gly;G), histidine (His;H), isoleucine (Ile;I), leucine (Leu;L), lysine (Lys;K), methionine (Met;M), phenylalanine (Phe;F), proline (Pro;P), serine (Ser;S), threonine (Thr;T), tryptophan (Trp;W), tyrosine (Tyr;Y), and valine (Val;V).

In the broadest sense, the naturally occurring amino acids can be divided into groups based upon the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His. This grouping of amino acids can be further subclassed as follows. By “uncharged hydrophilic” amino acid is meant either Ser, Thr, Asn or Gln. By “acidic” amino acid is meant either Glu or Asp. By “basic” amino acid is meant either Lys, Arg or His.

As used herein the term “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The term “oligonucleotide” as used herein refers to an oligomer or polymer of ribonucleic acid (RNA) or deoxyribonucleic acid (DNA) or mimetics thereof. This term also covers those oligonucleobases composed of naturally-occurring nucleotides, sugars and covalent internucleoside (backbone) linkages as well as oligonucleotides having non-naturally-occurring modifications.

II. The Alternative Pathway and the Biological Role of MAp19: a New Understanding

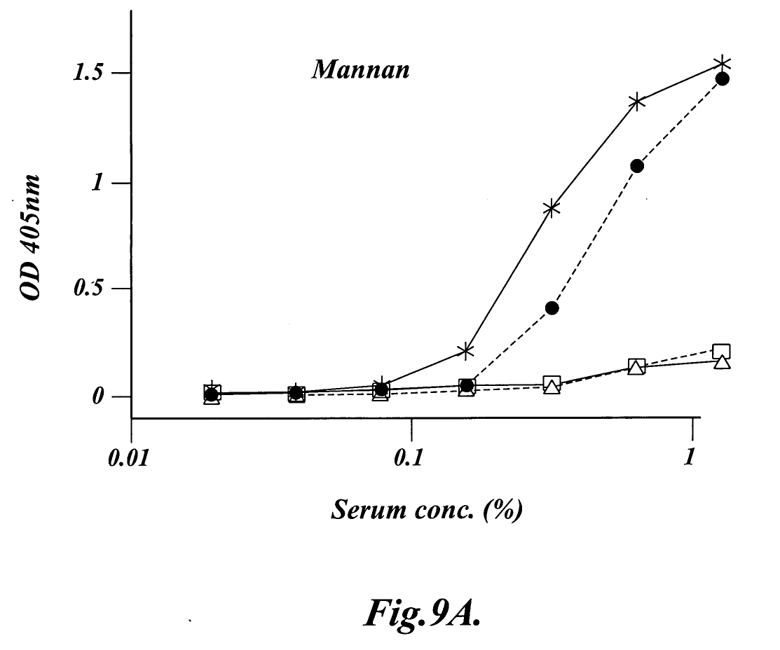

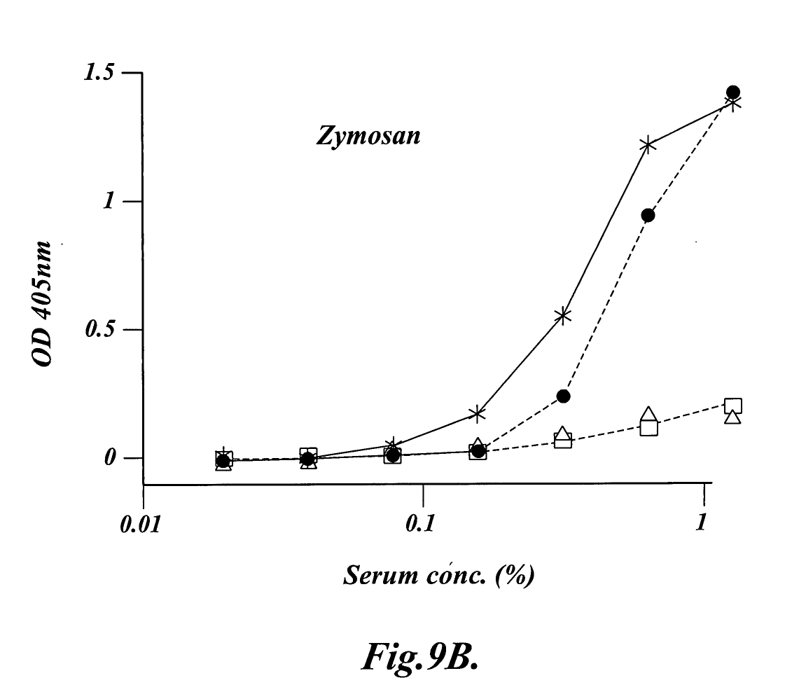

The alternative pathway of complement was first described by Louis Pillemer and his colleagues in the early 1950s based on studies in which zymosan, made from yeast cell walls, was used to activate complement (Pillemer, L. et al., J. Exp. Med. 103:1-13, 1956; Lepow, I. H., J. Immunol. 125:471-478, 1980). Ever since then, zymosan is considered as the canonical example of a specific activator of the alternative pathway in human and rodent serum (Lachmann, P. J., et al., Springer Semin. Immunopathol. 7:143-162, 1984; Dijk et al., J. Immunol. Methods 85:233-243, 1985; Pangburn, M. K., Methods in Enzymol. 162:639-653, 1988). A convenient and widely used assay for alternative pathway activation is to incubate serum with zymosan coated onto plastic wells and to determine the amount of C3b deposition onto the solid phase following the incubation. As expected, there is substantial C3b deposition onto zymosan-coated wells following incubation with normal mouse serum (FIG. 9B). However, incubation of serum from homozygous MASP-2 deficient mice with zymosan-coated wells results in a substantial reduction in C3b deposition compared to that of normal serum. Furthermore, use of serum from mice heterozygous for deficiency in the MASP 2 gene in this assay results in levels of C3b deposition that are intermediate between those obtained with serum from homozygous MASP-2-deficient mice and normal mouse serum. Parallel results are also obtained using wells coated with mannan, another polysaccharide known to activate the alternative pathway (FIG. 9A). Since the normal and MASP-2 deficient mice share the same genetic background, except for the MASP 2 gene, these unexpected results demonstrate that MASP-2 plays a significant role in activation of the alternative pathway.

These results provide strong evidence that the alternative pathway is not an independent, stand-alone pathway of complement activation, as described in essentially all current medical textbooks and recent review articles on complement. The current and widely-held scientific view is that the alternative pathway is activated on the surface of certain particulate targets (microbes, zymosan, rabbit erythrocytes) through the amplification of spontaneous “tick-over” C3 activation. However, the absence of significant alternative pathway activation in serum from MASP-2 knockout mice by two well-known ‘activators’ of the alternative pathway makes it unlikely that the “tick-over theory” describes an important physiological mechanism for complement activation.

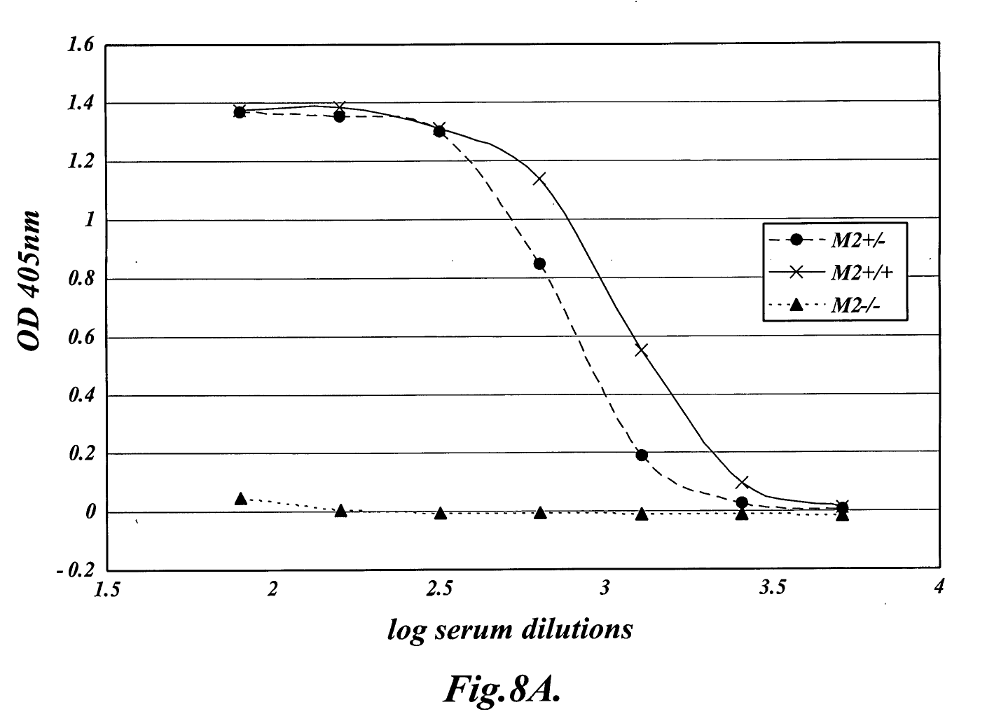

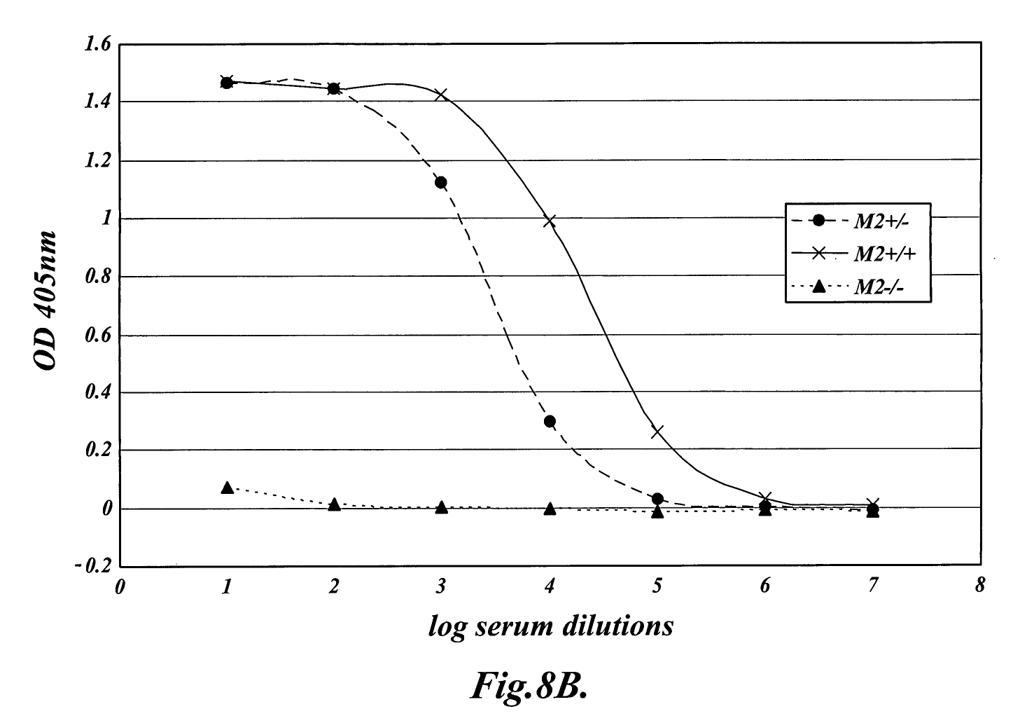

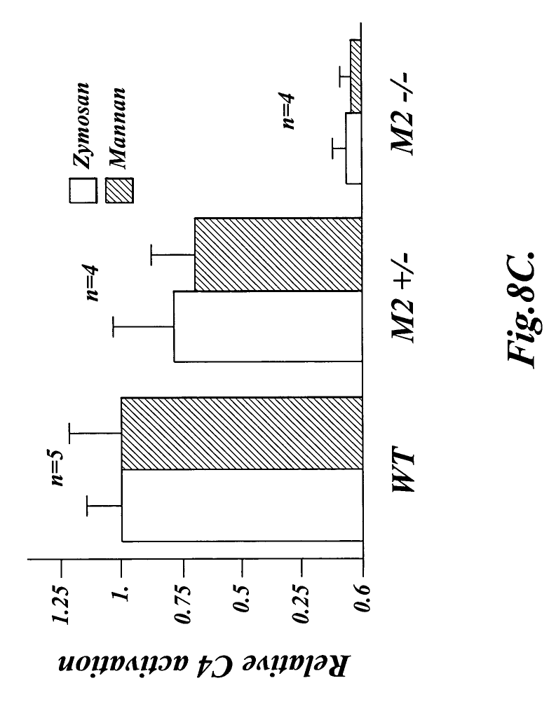

Since MASP-2 protease is known to have a specific and well-defined role as the enzyme responsible for the initiation of the lectin complement cascade, these results implicate activation of the lectin pathway by zymosan and mannan as a critical first step for subsequent activation of the alternative pathway. C4b is an activation product generated by the lectin pathway but not by the alternative pathway. Consistent with this concept, incubation of normal mouse serum with zymosan or mannan coated wells results in C4b deposition onto the wells, and this C4b deposition is substantially reduced when the coated wells are incubated with serum from MASP-2-deficient mice (FIGS. 8A, 8B and 8C).

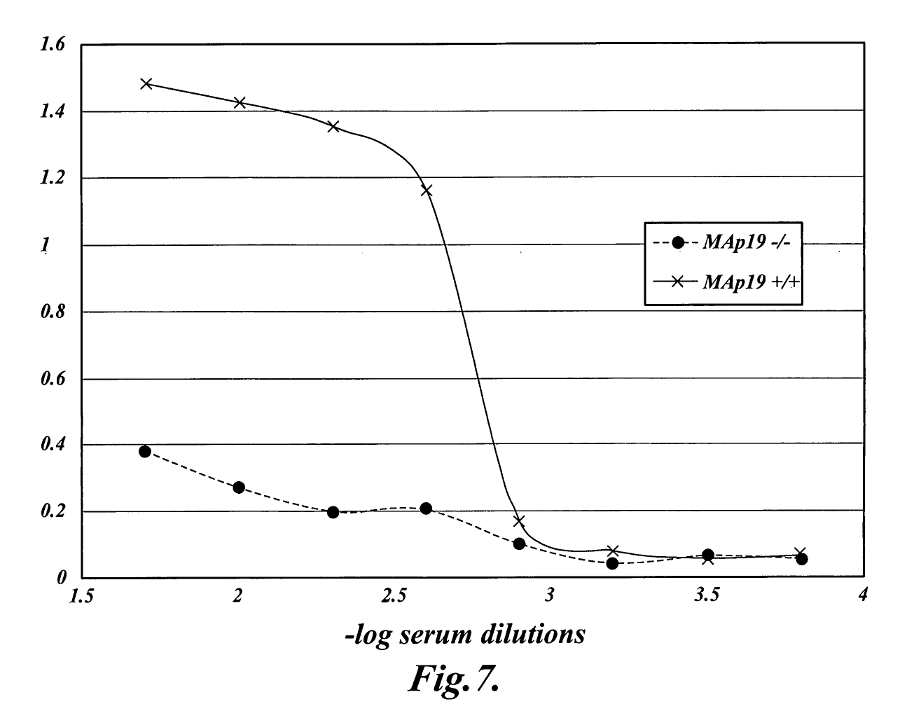

MAp19 is formed by alternative splicing of the MASP 2 gene product and comprises the first two domains of MASP-2, followed by an extra sequence of four unique amino acids. Incubation of normal mouse serum with zymosan or mannan coated wells results in C4b deposition onto the wells. The inventors have surprisingly found that C4b deposition is substantially reduced when the coated wells are incubated with serum from MAp19 deficient mice (FIG. 7), similar to the data for the MASP-2 deficient mice. We note, however, that the protein expression of MASP-2 is reduced in the MAp19 deficient mice, as determined by Western blot (data not shown). These results implicate MAp19 as having a biological role in the regulation of MASP-2 protein expression and activation, alone, or via MASP-2, of the lectin-dependent complement system. Activation of the lectin-dependant complement system may be regulated via MAp19 directly or through reduction of MASP-2 protein. This is even more surprising, given that alternative splicing in the generation of MAp19 results in the functional loss of serine protease activity previously thought to be critical to C4 cleavage and C4b deposition. Therefore, the results from the MAp19 knockout mouse indicate a role of MAp19 in the regulation and activation of the lectin pathway.

The alternative pathway, in addition to its widely accepted role as an independent pathway for complement activation, can also provide an amplification loop for complement activation initially triggered via the classical and lectin pathways (Liszewski, M. K. and J. P. Atkinson, in Fundamental Immunology, Third Edition, edited by W. E. Paul, Raven Press, Ltd., New York, 1993; Schweinie, J. E., et al., J. Clin. Invest. 84:1821-1829, 1989). In this alternative pathway-mediated amplification mechanism, C3 convertase (C4b2b), generated by activation of either the classical or lectin complement cascades, cleaves C3 into C3a and C3b, and thereby provides C3b that can participate in forming C3bBb, the alternative pathway C3 convertase. While not wishing to be limited by theory, the likely explanation for the absence of alternative pathway activation in MASP-2 knockout serum is that the lectin pathway is required for initial complement activation by zymosan, mannan, and other putative “activators” of the alternative pathway, while the alternative pathway plays a crucial role for amplifying complement activation. In other words, the alternative pathway is a feedforward amplification loop dependent upon the lectin and classical complement pathways for activation, rather than an independent linear cascade.

Rather than the complement cascade being activated through three distinct pathways (classical, alternative and lectin pathways) as previously envisioned, our results indicate that it is more accurate to view complement as being composed of two major systems, which correspond, to a first approximation, to the innate (lectin) and acquired (classical) wings of the complement immune defense system. Lectins (MBL, H-ficolin, L-ficolin, M-ficolin) are the specific recognition molecules that trigger the innate complement system. The innate complement system includes the lectin pathway and the associated alternative pathway amplification loop. C1q is the specific recognition molecule that triggers the acquired complement system. The acquired complement system includes the classical pathway and associated alternative pathway amplification loop. We refer to these two major complement activation systems as the lectin-dependent complement activation system and the C1q-dependent complement activation system, respectively.

The present invention further provides that MASP-2 is needed to initiate alternative complement pathway activation. Through the use of a knockout mouse model of MASP-2−/−, the present inventors have shown that it is possible to inhibit C3b deposition, the initiating step in alternative complement pathway activation via the lectin-dependent MASP-2 pathway, while leaving the classical pathway intact, thus establishing lectin-dependent MASP-2 activation as a requirement for alternative complement activation in the absence of classical pathway involvement. In another aspect of the invention, the establishment of lectin-dependent MASP-2 activation as a requirement for alternative complement activation in the absence of classical pathway involvement extends the present findings further to indicate that MAp19 may have a biological role in the regulation of MASP-2 protein expression and activity of the lectin dependent complement system. Through the use of a knockout mouse model of MAp19−/−, the present inventors have shown that it is possible to inhibit lectin pathway activation and C4 deposition via the lectin mediated MASP-2 pathway, while leaving the classical pathway intact. The present invention thus suggests the use of MAp19 as a therapeutic target for inhibiting cellular injury associated with lectin-mediated alternative complement pathway activation, while leaving the classical (C1q-dependent) pathway component of the immune system intact.

The first step in activation of the C1q dependent complement activation system is the binding of a specific recognition molecule, C1q, to antigen-bound IgG and IgM. The activation of the complement system results in the sequential activation of serine protease zymogens. C1q is associated with the C1r and C1s serine protease proenzymes as a complex called C1 and, upon binding of C1q to an immune complex, autoproteolytic cleavage of the Arg-Ile site of C1r is followed by C1r activation of C1s, which thereby acquires the ability to cleave C4 and C2. The cleavage of C4 into two fragments, designated C4a and C4b, allows the C4b fragments to form covalent bonds with adjacent hydroxyl or amino groups, and the subsequent generation of C3 convertase (C4b2b) through noncovalent interaction with the C2b fragment of activated C2. C3 convertase (C4b2b) activates C3 leading to generation of the C5 convertase (C4b2b3b) and formation of the membrane attack complex (C5b-9) that can cause microbial lysis. The activated forms of C3 and C4 (C3b and C4b) are covalently deposited on the foreign target surfaces, which are recognized by complement receptors on multiple phagocytes.

In addition to its essential role in immune defense, the complement system contributes to tissue damage in many clinical conditions. Thus, there is a pressing need to develop therapeutically effective complement inhibitors to prevent these adverse effects. With recognition that complement is composed of two major complement activation systems comes the realization that it would be highly desirable to specifically inhibit only the complement activation system, causing a particular pathology without completely shutting down the immune defense capabilities of complement. For example, in disease states in which complement activation is mediated predominantly by the lectin-dependent complement activation system, it would be advantageous to specifically inhibit only this system. This would leave the C1q-dependent complement activation system intact to handle immune complex processing and to aid in host defense against infection.

The preferred protein component to target in the development of therapeutic agents to specifically inhibit the lectin-dependent complement activation system is MASP-2 or MAp19. Of all the protein components of the lectin-dependent complement activation system (MBP, H-ficolin, L-ficolin, M-ficolin, MAp19, MASP-2, C2-C9, FactorB, FactorD, and properdin), MASP-2 and MAp19 are unique to the lectin-dependent complement activation system and required for the system to function. The lectins (MBL, H-ficolin, M-ficolin, and L-ficolin) are also unique components in the MASP-2-dependent complement activation system. However, loss of any one of the lectin components would not necessarily inhibit activation of the system due to lectin redundancy. It would be necessary to inhibit all four lectins in order to guarantee inhibition of the lectin-dependent complement activation system. Furthermore, because MBL and the ficolins are also known to have opsonic activity independent of complement, inhibition of lectin function would result in the loss of this beneficial host defense mechanism against infection. In contrast, this complement-independent lectin opsonic activity would remain intact if MASP-2 or MAp19 was the inhibitory target.

III. Role of Lectin-Dependent Complement Activation in Various Diseases and Conditions and Therapeutic Methods Using MAp19 Inhibitory Agents

Ischemia Reperfusion Injury

Ischemia reperfusion injury (I/R) occurs when blood flow is restored after an extended period of ischemia. It is a common source of morbidity and mortality in a wide spectrum of diseases. Surgical patients are vulnerable after aortic aneurysm repair, cardiopulmonary bypass, vascular reanastomosis in connection with, for example, organ transplants (e.g., heart, lung, liver, kidney) and digit/extremity replantation, stroke, myocardial infarction and hemodynamic resuscitation following shock and/or surgical procedures. Patients with atherosclerotic diseases are prone to myocardial infarctions, strokes, and emboli-induced intestinal and lower-extremity ischemia. Patients with trauma frequently suffer from temporary ischemia of the limbs. In addition, any cause of massive blood loss leads to a whole-body I/R reaction.

The pathophysiology of I/R injury is complex, with at least two major factors contributing to the process: complement activation and neutrophil stimulation with accompanying oxygen radical-mediated injury. In I/R injury, complement activation was first described during myocardial infarction over 30 years ago, and has led to numerous investigations on the contribution of the complement system to I/R tissue injury (Hill, J. H., et al., J. Exp. Med. 133:885-900, 1971). Accumulating evidence now points to complement as a pivotal mediator in I/R injury. Complement inhibition has been successful in limiting injury in several animal models of I/R. In early studies, C3 depletion was achieved following infusion of cobra venom factor, reported to be beneficial during I/R in kidney and heart (Maroko, P. R., et al., J. Clin. Invest. 61:661-670, 1978; Stein, S. H., et al., Miner. Electrolyte Metab. 11:256-61, 1985). However, the soluble form of complement receptor 1 (sCR1) was the first complement-specific inhibitor utilized for the prevention of myocardial I/R injury (Weisman, H. F., et al., Science 249:146-51, 1990). sCR1 treatment during myocardial I/R attenuates infarction associated with decreased deposition of C5b-9 complexes along the coronary endothelium and decreased leukocyte infiltration after reperfusion.

In experimental myocardial I/R, C1 esterase inhibitor (C1 INH) administered before reperfusion prevents deposition of C1q and significantly reduces the area of cardiac muscle necrosis (Buerke, M., et al., Circulation 91:393-402, 1995). Animals genetically deficient in C3 have less local tissue necrosis after skeletal muscle or intestinal ischaemia (Weiser, M. R., et al., J. Exp. Med. 183:2343-48, 1996).

The membrane attack complex is the ultimate vehicle of complement-directed injury and studies in C5-deficient animals have shown decreased local and remote injury in models of I/R injury (Austen, W. G. Jr., et al., Surgery 126:343-48, 1999). An inhibitor of complement activation, soluble Crry (complement receptor-related gene Y), has been shown to be effective against injury when given both before and after the onset of murine intestinal reperfusion (Rehrig, S., et al., J. Immunol. 167:5921-27, 2001). In a model of skeletal muscle ischemia, the use of soluble complement receptor 1 (sCR1) also reduced muscle injury when given after the start of reperfusion (Kyriakides, C., et al., Am. J. Physiol. Cell Physiol. 281:C244-30, 2001). In a porcine model of myocardial I/R, animals treated with monoclonal antibody (“MoAb”) to the anaphylatoxin C5a prior to reperfusion showed attenuated infarction (Amsterdam, E. A., et al., Am. J. Physiol. Heart Circ. Physiol. 268:H448-57, 1995). Rats treated with C5 MoAb demonstrated attenuated infarct size, neutrophil infiltration and apoptosis in the myocardium (Vakeva, A., et al., Circulation 97:2259-67, 1998). These experimental results highlight the importance of complement activation in the pathogenesis of I/R injury.

It is unclear which complement pathway (classical, lectin, or alternative) is predominantly involved in complement activation in I/R injury. Weiser et al. demonstrated an important role of the lectin and/or classical pathways during skeletal I/R by showing that C3- or C4-knockout mice were protected against I/R injury based on a significant reduction in vascular permeability (Weiser, M. R., et al., J. Exp. Med. 183:2343-48, 1996). In contrast, renal I/R experiments with C4 knockout mice demonstrate no significant tissue protection, while C3-, C5-, and C6-knockout mice were protected from injury, suggesting that complement activation during renal I/R injury occurs via the alternative pathway (Zhou, W., et al., J. Clin. Invest. 105:1363-71, 2000). Using factor D deficient mice, Stahl et al. recently presented evidence for an important role of the alternative pathway in intestinal I/R in mice (Stahl, G., et al., Am. J. Pathol. 162:449-55, 2003). In contrast, Williams et al. suggested a predominant role of the classical pathway for initiation of I/R injury in the intestine of mice by showing reduced organ staining for C3 and protection from injury in C4 and IgM (Rag1−/−) deficient mice (Williams, J. P., et al., J. Appl. Physiol. 86:938-42, 1999).

Treatment of rats in a myocardial I/R model with monoclonal antibodies against rat mannan-binding lectin (MBL) resulted in reduced postischemic reperfusion injury (Jordan, J. E., et al., Circulation 104:1413-18, 2001). MBL antibodies also reduced complement deposition on endothelial cells in vitro after oxidative stress indicating a role for the lectin pathway in myocardial I/R injury (Collard, C. D., et al., Am. J. Pathol. 156:1549-56, 2000). There is also evidence that R/I injury in some organs may be mediated by a specific category of IgM, termed natural antibodies, and activation of the classical pathway (Fleming, S. D., et al., J. Immunol. 169:2126-33, 2002; Reid, R. R., et al., J. Immunol. 169:5433-40, 2002).

Several inhibitors of complement activation have been developed as potential therapeutic agents to prevent morbidity and mortality resulting from myocardial I/R complications. Two of these inhibitors, sCR1 (TP10) and humanized anti-C5 scFv (Pexelizumab), have completed Phase II clinical trials. Pexelizumab has additionally completed a Phase III clinical trial. Although TP10 was well tolerated and beneficial to patients in early Phase I/II trials, results from a Phase II trial ending in February 2002 failed to meet its primary endpoint. However, sub-group analysis of the data from male patients in a high-risk population undergoing open-heart procedures demonstrated significantly decreased mortality and infarct size. Administration of a humanized anti-C5 scFv decreased overall patient mortality associated with acute myocardial infarction in the COMA and COMPLY Phase II trials, but failed to meet the primary endpoint (Mahaffey, K. W., et al., Circulation 108:1176-83, 2003). Results from a recent Phase III anti-C5 scFv clinical trial (PRIMO-CABG) for improving surgically induced outcomes following coronary artery bypass were recently released. Although the primary endpoint for this study was not reached, the study demonstrated an overall reduction in postoperative patient morbidity and mortality.

One aspect of the invention is thus directed to inhibiting lectin-dependent complement activation in the treatment of ischemia reperfusion injuries by administering a therapeutically effective amount of a MAp19 inhibitory agent in a pharmaceutical carrier to a subject experiencing ischemic reperfusion. The MAp19 inhibitory agent may be administered to the subject by intra-arterial, intravenous, intracranial, intramuscular, subcutaneous, or other parenteral administration, and potentially orally for non-peptidergic inhibitors, and most suitably by intra-arterial or intravenous administration. Administration of the MAp19 inhibitory compositions of the present invention suitably commences immediately after or as soon as possible after an ischemia reperfusion event. In instances where reperfusion occurs in a controlled environment (e.g., following an aortic aneurism repair, organ transplant or reattachment of severed or traumatized limbs or digits), the MAp19 inhibitory agent may be administered prior to and/or during and/or after reperfusion. Administration may be repeated periodically as determined by a physician for optimal therapeutic effect.

Atherosclerosis

There is considerable evidence that complement activation is involved in atherogenesis in humans. A number of studies have convincingly shown that, although no significant complement activation takes place in normal arteries, complement is extensively activated in atherosclerotic lesions and is especially strong in vulnerable and ruptured plaques. Components of the terminal complement pathway are frequently found in human atheromas (Niculescu, F. et al., Mol. Immunol. 36:949-55.10-12, 1999; Rus, H. G., et al., Immunol. Lett. 20:305-310, 1989; Torzewski, M., et al., Arterioscler. Thromb. Vasc. Biol. 18:369-378, 1998). C3 and C4 deposition in arterial lesions has also been demonstrated (Hansson, G. K., et al., Acta Pathol. Microbiol. Immunol. Scand. (A) 92:429-35, 1984). The extent of C5b-9 deposition was found to correlate with the severity of the lesion (Vlaicu, R., et al., Atherosclerosis 57:163-77, 1985). Deposition of complement iC3b, but not C5b-9, was especially strong in ruptured and vulnerable plaques, suggesting that complement activation may be a factor in acute coronary syndromes (Taskinen S., et al., Biochem. J. 367:403-12, 2002). In experimental atheroma in rabbits, complement activation was found to precede the development of lesions (Seifer, P. S., et al., Lab Invest. 60:747-54, 1989).

In atherosclerotic lesions, complement is activated via the classic and alternative pathways, but there is little evidence, as yet, of complement activation via the lectin pathway. Several components of the arterial wall may trigger complement activation. The classical pathway of complement may be activated by C-reactive protein (CRP) bound to enzymatically degraded LDL (Bhakdi, S., et al., Arterioscler. Thromb. Vasc. Biol. 19:2348-54, 1999). Consistent with this view is the finding that the terminal complement proteins colocalize with CRP in the intima of early human lesions (Torzewski, J., et al., Arterioscler. Thromb. Vasc. Biol. 18:1386-92, 1998). Likewise, immunoglobulin M or IgG antibodies specific for oxidized LDL within lesions may activate the classical pathway (Witztum, J. L., Lancet 344:793-95, 1994). Lipids isolated from human atherosclerotic lesions have a high content of unesterified cholesterol and are able to activate the alternative pathway (Seifert P. S., et al., J. Exp. Med. 172:547-57, 1990). Chlamydia pneumoniae, a Gram-negative bacteria frequently associated with atherosclerotic lesions, may also activate the alternative pathway of complement (Campbell L. A., et al., J. Infect. Dis. 172:585-8, 1995). Other potential complement activators present in atherosclerotic lesions include cholesterol crystals and cell debris, both of which can activate the alternative pathway (Seifert, P. S., et al., Mol. Immunol. 24:1303-08, 1987).

Byproducts of complement activation are known to have many biological properties that could influence the development of atherosclerotic lesions. Local complement activation may induce cell lysis and generate at least some of the cell debris found in the necrotic core of advanced lesions (Niculescu, F., et al., Mol. Immunol. 36:949-55.10-12, 1999). Sublytic complement activation could be a significant factor contributing to smooth muscle cell proliferation and to monocyte infiltration into the arterial intima during atherogenesis (Torzewski J., et al., Arterioscler. Thromb. Vasc. Biol. 18:673-77, 1996). Persistent activation of complement may be detrimental because it may trigger and sustain inflammation. In addition to the infiltration of complement components from blood plasma, arterial cells express messenger RNA for complement proteins and the expression of various complement components is upregulated in atherosclerotic lesions (Yasojima, K., et al., Arterioscler. Thromb. Vasc. Biol. 21:1214-19, 2001).

A limited number of studies on the influence of complement protein deficiencies on atherogenesis have been reported. The results in experimental animal models have been conflicting. In the rat, the formation of atherosclerotic-like lesions induced by toxic doses of vitamin D was diminished in complement-depleted animals (Geertinger, P., et al., Acta. Pathol. Microbiol Scand. (A) 78:284-88, 1970). Furthermore, in cholesterol-fed rabbits, complement inhibition either by genetic C6 deficiency (Geertinger, P., et al., Artery 1:177-84, 1977; Schmiedt, W., et al., Arterioscl. Thromb. Vasc. Biol. 18:1790-1795, 1998) or by anticomplement agent K-76 COONa (Saito, E., et al., J. Drug Dev. 3:147-54, 1990) suppressed the development of atherosclerosis without affecting the serum cholesterol levels. In contrast, a recent study reported that C5 deficiency does not reduce the development of atherosclerotic lesions in apolipoprotein E (ApoE) deficient mice (Patel, S., et al., Biochem. Biophys. Res. Commun. 286:164-70, 2001). However, in another study the development of atherosclerotic lesions in LDLR-deficient (ldlr-) mice with or without C3 deficiency was evaluated (Buono, C., et al., Circulation 105:3025-31, 2002). They found that the maturation of atheromas to atherosclerotic-like lesions depends in part of the presence of an intact complement system.

One aspect of the invention is thus directed to the treatment or prevention of atherosclerosis by inhibiting lectin-dependent complement activation by treating a subject suffering from or prone to atherosclerosis with a therapeutically effective amount of a MAp19 inhibitory agent in a pharmaceutical carrier. The MAp19 inhibitory agent may be administered to the subject by intra-arterial, intravenous, intrathecal, intracranial, intramuscular, subcutaneous or other parenteral administration, and potentially orally for non-peptidergic inhibitors. Administration of the MAp19 inhibitory composition may commence after diagnosis of atherosclerosis in a subject or prophylactically in a subject at high risk of developing such a condition. Administration may be repeated periodically as determined by a physician for optimal therapeutic effect.

Other Vascular Diseases and Conditions

The endothelium is largely exposed to the immune system and is particularly vulnerable to complement proteins that are present in plasma. Complement-mediated vascular injury has been shown to contribute to the pathophysiology of several diseases of the cardiovascular system, including atherosclerosis (Seifert, P. S., et al., Atherosclerosis 73:91-104, 1988), ischemia-reperfusion injury (Weisman, H. F., Science 249:146-51, 1990) and myocardial infarction (Tada, T., et al., Virchows Arch. 430:327-332, 1997). Evidence suggests that complement activation may extend to other vascular conditions.

For example, there is evidence that complement activation contributes to the pathogenesis of many forms of vasculitis, including: Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease. Henoch-Schonlein purpura nephritis is a form of systemic vasculitis of the small vessels with immune pathogenesis, in which activation of complement through the lectin pathway leading to C5b-9-induced endothelial damage is recognized as an important mechanism (Kawana, S., et al., Arch. Dermatol. Res. 282:183-7, 1990; Endo, M., et al., Am. J. Kidney Dis. 35:401-7, 2000). Systemic lupus erythematosus (SLE) is an example of systemic autoimmune diseases that affects multiple organs, including skin, kidneys, joints, serosal surfaces, and central nervous system, and is frequently associated with severe vasculitis. IgG anti-endothelial antibodies and IgG complexes capable of binding to endothelial cells are present in the sera of patients with active SLE, and deposits of IgG immune complexes and complement are found in blood vessel walls of patients with SLE vasculitis (Cines, D. B., et al., J. Clin. Invest. 73:611-25, 1984). Rheumatoid arthritis associated with vasculitis, also called malignant rheumatoid arthritis (Tomooka, K., Fukuoka Igaku Zasshi 80:456-66, 1989), immune-complex vasculitis, vasculitis associated with hepatitis A, leukocytoclastic vasculitis, and the arteritis known as Takayasu's disease form another pleomorphic group of human diseases in which complement-dependent cytotoxicity against endothelial and other cell types plays a documented role (Tripathy, N. K., et al., J. Rheumatol. 28:805-8, 2001).

Evidence also suggests that complement activation plays a role in dilated cardiomyopathy. Dilated cardiomyopathy is a syndrome characterized by cardiac enlargement and impaired systolic function of the heart. Recent data suggests that ongoing inflammation in the myocardium may contribute to the development of disease. C5b-9, the terminal membrane attack complex of complement, is known to significantly correlate with immunoglobulin deposition and myocardial expression of TNF-alpha. In myocardial biopsies from 28 patients with dilated cardiomyopathy, myocardial accumulation of C5b-9 was demonstrated, suggesting that chronic immunoglobulin-mediated complement activation in the myocardium may contribute in part to the progression of dilated cardiomyopathy (Zwaka, T. P., et al., Am. J. Pathol. 161(2):449-57, 2002).

One aspect of the invention is thus directed to the treatment of a vascular condition, including cardiovascular conditions, cerebrovascular conditions, peripheral (e.g., musculoskeletal) vascular conditions, renovascular conditions, and mesenteric/enteric vascular conditions, by inhibiting lectin-dependent complement activation by administering a composition comprising a therapeutically effective amount of a MAp19 inhibitory agent in a pharmaceutical carrier. Conditions for which the invention is believed to be suited include, without limitation: vasculitis, including Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); and venous gas embolus (VGE). Also, given that complement activation occurs as a result of luminal trauma and the foreign-body inflammatory response associated with cardiovascular interventional procedures, it is believed that the MAp19 inhibitory compositions of the present invention may also be used in the inhibition of restenosis following stent placement, rotational atherectomy and/or percutaneous transluminal coronary angioplasty (PTCA), either alone or in combination with other restenosis inhibitory agents such as are disclosed in U.S. Pat. No. 6,492,332 to Demopulos.

The MAp19 inhibitory agent may be administered to the subject by intra-arterial, intravenous, intramuscular, intrathecal, intracranial, subcutaneous or other parenteral administration, and potentially orally for non-peptidergic inhibitors. Administration may be repeated periodically as determined by a physician for optimal therapeutic effect. For the inhibition of restenosis, the MAp19 inhibitory composition may be administered before and/or during and/or after the placement of a stent or the atherectomy or angioplasty procedure. Alternately, the MAp19 inhibitory composition may be coated on or incorporated into the stent.

Gastrointestinal Disorders

Ulcerative colitis and Crohn's disease are chronic inflammatory disorders of the bowel that fall under the banner of inflammatory bowel disease (IBD). IBD is characterized by spontaneously occurring, chronic, relapsing inflammation of unknown origin. Despite extensive research into the disease in both humans and experimental animals, the precise mechanisms of pathology remain to be elucidated. However, the complement system is believed to be activated in patients with IBD and is thought to play a role in disease pathogenesis (Kolios, G., et al., Hepato-Gastroenterology 45:1601-9, 1998; Elmgreen, J., Dan. Med. Bull. 33:222, 1986).

It has been shown that C3b and other activated complement products are found at the luminal face of surface epithelial cells, as well as in the muscularis mucosa and submucosal blood vessels in IBD patients (Halstensen, T. S., et al., Immunol. Res. 10:485-92, 1991; Halstensen, T. S., et al., Gastroenterology 98:1264, 1990). Furthermore, polymorphonuclear cell infiltration, usually a result of C5a generation, characteristically is seen in the inflammatory bowel (Kohl, J., Mol. Immunol. 38:175, 2001). The multifunctional complement inhibitor K-76, has also been reported to produce symptomatic improvement of ulcerative colitis in a small clinical study (Kitano, A., et al., Dis. Colon Rectum 35:560, 1992), as well as in a model of carrageenan-induced colitis in rabbits (Kitano, A., et al., Clin. Exp. Immunol. 94:348-53, 1993).

A novel human C5a receptor antagonist has been shown to protect against disease pathology in a rat model of IBD (Woodruff, T. M., et al., J. Immunol. 171:5514-20, 2003). Mice that were genetically deficient in decay-accelerating factor (DAF), a membrane complement regulatory protein, were used in a model of IBD to demonstrate that DAF deficiency resulted in markedly greater tissue damage and increased proinflammatory cytokine production (Lin, F., et al., J. Immunol. 172:3836-41, 2004). Therefore, control of complement is important in regulating gut homeostasis and may be a major pathogenic mechanism involved in the development of IBD.

The present invention thus provides methods for inhibiting lectin-dependent complement activation in subjects suffering from inflammatory gastrointestinal disorders, including, but not limited to: pancreatitis, diverticulitis and bowel disorders, including Crohn's disease, ulcerative colitis, and irritable bowel syndrome, by administering a composition comprising a therapeutically effective amount of a MAp19 inhibitory agent in a pharmaceutical carrier to a patient suffering from such a disorder. The MAp19 inhibitory agent may be administered to the subject by intra-arterial, intravenous, intramuscular, subcutaneous, intrathecal, intracranial or other parenteral administration, and potentially orally for non-peptidergic inhibitors. Administration may suitably be repeated periodically as determined by a physician to control symptoms of the disorder being treated.

Pulmonary Conditions

Complement has been implicated in the pathogenesis of many lung inflammatory disorders, including: acute respiratory distress syndrome (ARDS) (Ware, I., et al., N. Engl. J. Med. 342:1334-49, 2000); transfusion-related acute lung injury (TRALI) (Seeger, W., et al., Blood 76:1438-44, 1990); ischemia/reperfusion acute lung injury (Xiao, F., et al., J. Appl. Physiol. 82:1459-65, 1997); chronic obstructive pulmonary disease (COPD) (Marc, M. M., et al., Am. J. Respir. Cell Mol. Biol. (Epub ahead of print), Mar. 23, 2004); asthma (Krug, N., et al., Am. J. Respir. Crit. Care Med. 164:1841-43, 2001); Wegener's granulomatosis (Kalluri, R., et al., J. Am. Soc. Nephrol. 8:1795-800, 1997); and antiglomerular basement membrane disease (Goodpasture's disease) (Kondo, C., et al., Clin. Exp. Immunol. 124:323-9, 2001).

It is now well accepted that much of the pathophysiology of ARDS involves a dysregulated inflammatory cascade that begins as a normal response to an infection or other inciting event, but ultimately causes significant autoinjury to the host (Stanley, T. P., Emerging Therapeutic Targets 2:1-16, 1998). Patients with ARDS almost universally show evidence of extensive complement activation (increased plasma levels of complement components C3a and C5a), and the degree of complement activation has been correlated with the development and outcome of ARDS (Hammerschmidt, D. F., et al., Lancet 1:947-49, 1980; Solomkin, J. S., et al., J. Surgery 97:668-78, 1985).

Various experimental and clinical data suggest a role for complement activation in the pathophysiology of ARDS. In animal models, systemic activation of complement leads to acute lung injury with histopathology similar to that seen in human ARDS (Till, G. O., et al., Am. J. Pathol. 129:44-53, 1987; Ward, P. A., Am. J. Pathol. 149:1081-86, 1996). Inhibiting the complement cascade by general complement depletion or by specific inhibition of C5a confers protection in animal models of acute lung injury (Mulligan, M. S., et al., J. Clin. Invest. 98:503-512, 1996). In rat models, sCR1 has a protective effect in complement- and neutrophil-mediated lung injury (Mulligan, M. S., et al., J. Immunol. 148:1479-85, 1992). In addition, virtually all complement components can be produced locally in the lung by type II alveolar cells, alveolar macrophages and lung fibroblasts (Hetland, G., et al., Scand. J. Immunol. 24:603-8, 1986; Rothman, B. I., et al., J. Immunol. 145:592-98, 1990). Thus, the complement cascade is well positioned to contribute significantly to lung inflammation and, consequently, to lung injury in ARDS.

Asthma is, in essence, an inflammatory disease. The cardinal features of allergic asthma include airway hyperresponsiveness to a variety of specific and nonspecific stimuli, excessive airway mucus production, pulmonary eosinophilia, and elevated concentration of serum IgE. Although asthma is multifactorial in origin, it is generally accepted that it arises as a result of inappropriate immunological responses to common environmental antigens in genetically susceptible individuals. The fact that the complement system is highly activated in the human asthmatic lung is well documented (Humbles, A. A., et al., Nature 406:998-01, 2002; van de Graf, E. A., et al., J. Immunol. Methods 147:241-50, 1992). Furthermore, recent data from animal models and humans provide evidence that complement activation is an important mechanism contributing to disease pathogenesis (Karp, C. L., et al., Nat. Immunol. 1:221-26, 2000; Bautsch, W., et al., J. Immunol. 165:5401-5, 2000; Drouin, S. M., et al., J. Immunol. 169:5926-33, 2002; Walters, D. M., et al., Am. J. Respir. Cell Mol. Biol. 27:413-18, 2002). A role for the lectin pathway in asthma is supported by studies using a murine model of chronic fungal asthma. Mice with a genetic deficiency in mannan-binding lectin develop an altered airway hyperresponsiveness compared to normal animals in this asthma model (Hogaboam, C. M., et al., J. Leukoc. Biol. 75:805-14, 2004).

Complement may be activated in asthma via several pathways, including:
(a) activation through the classical pathway as a result of allergen-antibody complex formation; (b) alternative pathway activation on allergen surfaces; (c) activation of the lectin pathway through engagement of carbohydrate structures on allergens; and (d) cleavage of C3 and C5 by proteases released from inflammatory cells. Although much remains to be learned about the complex role played by complement in asthma, identification of the complement activation pathways involved in the development of allergic asthma may provide a focus for development of novel therapeutic strategies for this increasingly important disease.

An aspect of the invention thus provides a method for treating pulmonary disorders, by administering a composition comprising a therapeutically effective amount of a MAp19 inhibitory agent in a pharmaceutical carrier to a subject suffering from a pulmonary disorder, including, without limitation, acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, and emphysema. The MAp19 inhibitory agent may be administered to the subject systemically, such as by intra-arterial, intravenous, intramuscular, inhalational, nasal, subcutaneous, or other parenteral administration, or potentially by oral administration for non-peptidergic agents. The MAp19 inhibitory agent composition may be combined with one or more additional therapeutic agents, including anti-inflammatory agents, antihistamines, corticosteroids or antimicrobial agents. Administration may be repeated as determined by a physician until the condition has been resolved.

Extracorporeal Circulation

There are numerous medical procedures during which blood is diverted from a patient's circulatory system (extracorporeal circulation systems or ECC). Such procedures include hemodialysis, plasmapheresis, leukopheresis, extracorporeal membrane oxygenator (ECMO), heparin-induced extracorporeal membrane oxygenation LDL precipitation (HELP), and cardiopulmonary bypass (CPB). These procedures expose blood or blood products to foreign surfaces that may alter normal cellular function and hemostasis. In pioneering studies, Craddock et al. identified complement activation as the probable cause of granulocytopenia during hemodialysis (Craddock, P. R., et al., N. Engl. J. Med. 296:769-74, 1977). The results of numerous studies between 1977 and the present time indicate that many of the adverse events experienced by patients undergoing hemodialysis or CPB are caused by activation of the complement system (Chenoweth, D. E., Ann. N.Y. Acad. Sci. 516:306-313, 1987; Hugli, T. E., Complement 3:111-127, 1986; Cheung, A. K., J. Am. Soc. Nephrol. 1:150-161, 1990; Johnson, R. J., Nephrol. Dial. Transplant 9:36-45, 1994). For example, the complement activating potential has been shown to be an important criterion in determination of the biocompatibility of hemodialyzers with respect to recovery of renal function, susceptibility to infection, pulmonary dysfunction, morbidity, and survival rate of patients with renal failure (Hakim, R. M., Kidney Int. 44:484-4946, 1993).

It has been largely believed that complement activation by hemodialysis membranes occurs by alternative pathway mechanisms due to weak C4a generation (Kirklin, J. K., et al., J. Thorac. Cardiovasc. Surg. 86:845-57, 1983; Vallhonrat, H., et al., ASAIO J. 45:113-4, 1999), but recent work suggests that the classical pathway may also be involved (Wachtfogel, Y. T., et al., Blood 73:468-471, 1989). However, there is still inadequate understanding of the factors initiating and controlling complement activation on artificial surfaces including biomedical polymers. For example, Cuprophan membrane used in hemodialysis has been classified as a very potent complement activator. While not wishing to be limited by theory, the inventors theorize that this could perhaps be explained in part by its polysaccharide nature. The MASP-2-dependent complement activation system identified in this patent provides a mechanism whereby activation of the lectin pathway triggers alternative pathway activation.

Patients undergoing ECC during CPB suffer a systemic inflammatory reaction, which is partly caused by exposure of blood to the artificial surfaces of the extracorporeal circuit, but also by surface-independent factors like surgical trauma and ischemia-reperfusion injury (Butler, J., et al., Ann. Thorac. Surg. 55:552-9, 1993; Edmunds, L. H., Ann. Thorac. Surg. 66(Suppl):S12-6, 1998; Asimakopoulos, G., Perfusion 14:269-77, 1999). The CPB-triggered inflammatory reaction can result in postsurgical complications, generally termed “postperfusion syndrome.” Among these postoperative events are cognitive deficits (Fitch, J., et al., Circulation 100(25):2499-2506, 1999), respiratory failure, bleeding disorders, renal dysfunction and, in the most severe cases, multiple organ failure (Wan, S., et al., Chest 112:676-692, 1997). Coronary bypass surgery with CPB leads to profound activation of complement, in contrast to surgery without CPB but with a comparable degree of surgical trauma (E. Fosse, 1987). Therefore, the primary suspected cause of these CPB-related problems is inappropriate activation of complement during the bypass procedure (Chenoweth, K., et al., N. Engl. J. Med. 304:497-503, 1981; Haslam, P., et al., Anaesthesia 25:22-26, 1980; Kirklin, J. K., et al., J. Thorac. Cardiovasc. Surg. 86:845-857, 1983; Moore, F. D., et al., Ann. Surg. 208:95-103, 1988; Steinberg, J., et al., J. Thorac. Cardiovasc. Surg 106:1901-1918, 1993). In CPB circuits, the alternative complement pathway plays a predominant role in complement activation, resulting from the interaction of blood with the artificial surfaces of the CPB circuits (Kirklin, J. K., et al., J. Thorac. Cardiovasc. Surg., 86:845-57, 1983; Kirklin, J. K., et al., Ann. Thorac. Surg. 41:193-199, 1986; Vallhonrat, H., et al., ASAIO J. 45:113-4, 1999). However, there is also evidence that the classical complement pathway is activated during CPB (Wachtfogel, Y. T., et al., Blood 73:468-471, 1989).

Primary inflammatory substances are generated after activation of the complement system, including anaphylatoxins C3a and C5a, the opsonin C3b, and the membrane attack complex C5b-9. C3a and C5a are potent stimulators of neutrophils, monocytes, and platelets (Haeffner-Cavaillon, N., et al., J. Immunol., 139:794-9, 1987; Fletcher, M. P., et al., Am. J. Physiol. 265:H1750-61, 1993; Rinder, C. S., et al., J. Clin. Invest. 96:1564-72, 1995; Rinder, C. S., et al., Circulation 100:553-8, 1999). Activation of these cells results in release of proinflammatory cytokines (IL-1, IL-6, IL-8, TNF alpha), oxidative free radicals and proteases (Schindler, R., et al., Blood 76:1631-8, 1990; Cruickshank, A. M., et al., Clin. Sci. (Lond). 79:161-5, 1990; Kawamura, T., et al., Can. J. Anaesth. 40:1016-21, 1993; Steinberg, J. B., et al., J. Thorac. Cardiovasc. Surg. 106:1008-1, 1993; Finn, A., et al., J. Thorac. Cardiovasc. Surg. 105:234-41, 1993; Ashraf, S. S., et al., J. Cardiothorac. Vasc. Anesth. 11:718-22, 1997). C5a has been shown to upregulate adhesion molecules CD11b and CD18 of Mac-1 in polymorphonuclear cells (PMNs) and to induce degranulation of PMNs to release proinflammatory enzymes (Rinder, C., et al., Cardiovasc. Pharmacol. 27:Suppl 1:S6-12, 1996; Evangelista, V., et al., Blood 93:876-85, 1999; Kinkade, J. M. Jr., et al., Biochem. Biophys. Res. Commun. 114:296-303, 1983; Lamb, N. J., et al., Crit. Care Med. 27:1738-44, 1999; Fujie, K., et al., Eur. J. Pharmacol. 374:117-25, 1999). C5b-9 induces the expression of adhesion molecule P-selectin (CD62P) on platelets (Rinder, C. S., et al., J. Thorac. Cardiovasc. Surg. 118:460-6, 1999), whereas both C5a and C5b-9 induce surface expression of P-selectin on endothelial cells (Foreman, K. E., et al., J. Clin. Invest. 94:1147-55, 1994). These adhesion molecules are involved in the interaction among leukocytes, platelets and endothelial cells. The expression of adhesion molecules on activated endothelial cells is responsible for sequestration of activated leukocytes, which then mediate tissue inflammation and injury (Evangelista, V., Blood 1999; Foreman, K. E., J. Clin. Invest. 1994; Lentsch, A. B., et al., J. Pathol. 190:343-8, 2000). It is the actions of these complement activation products on neutrophils, monocytes, platelets, and other circulatory cells that likely lead to the various problems that arise after CPB.

Several complement inhibitors are being studied for potential applications in CPB. They include a recombinant soluble complement receptor 1 (sCR1) (Chai, P. J., et al., Circulation 101:541-6, 2000), a humanized single chain anti-C5 antibody (h5G1.1-scFv or Pexelizumab) (Fitch, J. C. K., et al., Circulation 100:3499-506, 1999), a recombinant fusion hybrid (CAB-2) of human membrane cofactor protein and human decay accelerating factor (Rinder, C. S., et al., Circulation 100:553-8, 1999), a 13-residue C3-binding cyclic peptide (Compstatin) (Nilsson, B., et al., Blood 92:1661-7, 1998) and an anti-factor D MoAb (Fung, M., et al., J. Thoracic Cardiovasc. Surg. 122:113-22, 2001). SCR1 and CAB-2 inhibit the classical and alternative complement pathways at the steps of C3 and C5 activation. Compstatin inhibits both complement pathways at the step of C3 activation, whereas h5G1.1-scFv does so only at the step of C5 activation. Anti-factor D MoAb inhibits the alternative pathway at the steps of C3 and C5 activation. However, none of these complement inhibitors would specifically inhibit the MASP-2-dependent complement activation system identified in this patent.

Results from a large prospective phase 3 clinical study to investigate the efficacy and safety of the humanized single chain anti-C5 antibody (h5G1.1-scFv, pexelizu mab) in reducing perioperative MI and mortality in coronary artery bypass graft (CABG) surgery has been reported (Verrier, E. D., et al., JAMA 291:2319-27, 2004). Compared with placebo, pexelizu mab was not associated with a significant reduction in the risk of the composite end point of death or MI in 2746 patients who had undergone CABG surgery. However, there was a statistically significant reduction 30 days after the procedure among all 3099 patients undergoing CABG surgery with or without valve surgery. Since pexelizu mab inhibits at the step of C5 activation, it inhibits C5a and sC5b-9 generation but has no effect on generation of the other two potent complement inflammatory substances, C3a and opsonic C3b, which are also known to contribute to the CPB-triggered inflammatory reaction.

One aspect of the invention is thus directed to the prevention or treatment of extracorporeal exposure-triggered inflammatory reaction by treating a subject undergoing an extracorporeal circulation procedure with a composition comprising a therapeutically effective amount of a MAp19 inhibitory agent in a pharmaceutical carrier, including patients undergoing hemodialysis, plasmapheresis, leukopheresis, extracorporeal membrane oxygenation (ECMO), heparin-induced extracorporeal membrane oxygenation LDL precipitation (HELP), and cardiopulmonary bypass (CPB). MAp19 inhibitory agent treatment in accordance with the methods of the present invention is believed to be useful in reducing or preventing the cognitive dysfunction that sometimes results from CPB procedures. The MAp19 inhibitory agent may be administered to the subject preprocedurally and/or intraprocedurally and/or postprocedurally, such as by intra-arterial, intravenous, intramuscular, subcutaneous, or other parenteral administration. Alternately, the MAp19 inhibitory agent may be introduced to the subject's bloodstream during extracorporeal circulation, such as by injecting the MAp19 inhibitory agent into tubing or a membrane through or past which the blood is circulated or by contacting the blood with a surface that has been coated with the MAp19 inhibitory agent such as an interior wall of the tubing, membrane or other surface such as a CPB device.

Inflammatory and Non-Inflammatory Arthritides and Other Musculoskeletal Diseases

Activation of the complement system has been implicated in the pathogenesis of a wide variety of rheumatological diseases, including: rheumatoid arthritis (Linton, S. M., et al., Molec. Immunol. 36:905-14, 1999), juvenile rheumatoid arthritis (Mollnes, T. E., et al., Arthritis Rheum. 29:1359-64, 1986), osteoarthritis (Kemp, P. A., et al., J. Clin. Lab. Immunol. 37:147-62, 1992), systemic lupus erythematosis (SLE) (Molina, H., Current Opinion in Rheumatol. 14:492-497, 2002), Behcet's syndrome (Rumfeld, W. R., et al., Br. J. Rheumatol. 25:266-70, 1986) and Sjogren's syndrome (Sanders, M. E., et al., J. Immunol. 138:2095-9, 1987).

There is compelling evidence that immune-complex-triggered complement activation is a major pathological mechanism that contributes to tissue damage in rheumatoid arthritis (RA). There are numerous publications documenting that complement activation products are elevated in the plasma of RA patients (Morgan, B. P., et al., Clin. Exp. Immunol, 73:473-478, 1988; Auda, G., et al., Rheumatol. Int. 10:185-189, 1990; Rumfeld, W. R., et al., Br. J. Rheumatol. 25:266-270, 1986). Complement activation products such as C3a, C5a, and sC5b-9 have also been found within inflamed rheumatic joints and positive correlations have been established between the degree of complement activation and the severity of RA (Makinde, V. A., et al., Ann. Rheum. Dis. 48:302-306, 1989; Brodeur, J. P., et al., Arthritis Rheumatism 34:1531-1537, 1991). In both adult and juvenile rheumatoid arthritis, elevated serum and synovial fluid levels of alternative pathway complement activation product Bb compared to C4d (a marker for classical pathway activation), indicate that complement activation is mediated predominantly by the alternative pathway (El-Ghobarey, A. F. et al., J. Rheumatology 7:453-460, 1980; Agarwal, A., et al., Rheumatology 39:189-192, 2000). Complement activation products can directly damage tissue (via C5b-9) or indirectly mediate inflammation through recruitment of inflammatory cells by the anaphylatoxins C3a and C5a.

Animal models of experimental arthritis have been widely used to investigate the role of complement in the pathogenesis of RA. Complement depletion by cobra venom factor in animal models of RA prevents the onset of arthritis (Morgan, K., et al., Arthritis Rheumat. 24:1356-1362, 1981; Van Lent, P. L., et al., Am. J. Pathol. 140:1451-1461, 1992). Intra-articular injection of the soluble form of complement receptor 1 (sCR1), a complement inhibitor, suppressed inflammation in a rat model of RA (Goodfellow, R. M., et al., Clin. Exp. Immunol. 110:45-52, 1997). Furthermore, sCR1 inhibits the development and progression of rat collagen-induced arthritis (Goodfellow, R. M., et al., Clin Exp. Immunol. 119:210-216, 2000). Soluble CR1 inhibits the classical and alternative complement pathways at the steps of C3 and C5 activation in both the alternative pathway and the classical pathway, thereby inhibiting generation of C3a, C5a and sC5b-9.

In the late 1970s it was recognized that immunization of rodents with heterologous type II collagen (CII; the major collagen component of human joint cartilage) led to the development of an autoimmune arthritis (collagen-induced arthritis, or CIA) with significant similarities to human RA (Courtenay, J. S., et al., Nature 283:666-68, 1980, Banda et al., J. Immunol. 171:2109-2115, 2003). The autoimmune response in susceptible animals involves a complex combination of factors including specific major histocompatability complex (MHC) molecules, cytokines and CII-specific B- and T-cell responses (reviewed by Myers, L. K., et al., Life Sciences 61:1861-78, 1997). The observation that almost 40% of inbred mouse strains have a complete deficiency in complement component C5 (Cinader, B., et al., J. Exp. Med. 120:897-902, 1964) has provided an indirect opportunity to explore the role of complement in this arthritic model by comparing CIA between C5-deficient and sufficient strains. Results from such studies indicate that C5 sufficiency is an absolute requirement for the development of CIA (Watson et al., 1987; Wang, Y., et al., J. Immunol. 164:4340-4347, 2000). Further evidence of the importance of C5 and complement in RA has been provided by the use of anti-C5 monoclonal antibodies (MoAbs). Prophylactic intraperitoneal administration of anti-C5 MoAbs in a murine model of CIA almost completely prevented disease onset while treatment during active arthritis resulted in both significant clinical benefit and milder histological disease (Wang, Y., et al., Proc. Natl. Acad. Sci. USA 92:8955-59, 1995).

Additional insights about the potential role of complement activation in disease pathogenesis have been provided by studies using K/B×N T-cell receptor transgenic mice, a recently developed model of inflammatory arthritis (Korganow, A. S., et al., Immunity 10:451-461, 1999). All K/B×N animals spontaneously develop an autoimmune disease with most (although not all) of the clinical, histological, and immunological features of RA in humans. Furthermore, transfer of serum from arthritic K/B×N mice into healthy animals provokes arthritis within days via the transfer of arthritogenic immunoglobulins. To identify the specific complement activation steps required for disease development, serum from arthritic K/B×N mice was transferred into various mice genetically deficient for a particular complement pathway product (Ji, H., et al., Immunity 16:157-68, 2002). Interestingly, the results of the study demonstrated that alternative pathway activation is critical, whereas classical pathway activation is dispensable. In addition, the generation of C5a is critical since both C5-deficient mice and C5aR-deficient mice were protected from disease development. Consistent with these results, a previous study reported that genetic ablation of C5a receptor expression protects mice from arthritis (Grant, E. P., et al., J. Exp. Med. 196:1461-1471, 2002).

A humanized anti-C5 MoAb (5G1.1) that prevents the cleavage of human complement component C5 into its proinflammatory components is under development by Alexion Pharmaceuticals, Inc., New Haven, Conn., as a potential treatment for RA.

Systemic lupus erythematosus (SLE) is an autoimmune disease of undefined etiology that results in production of autoantibodies, generation of circulating immune complexes, and episodic, uncontrolled activation of the complement system. Although the origins of autoimmunity in SLE remain elusive, considerable information is now available implicating complement activation as an important mechanism contributing to vascular injury in this disease (Abramson, S. B., et al., Hospital Practice 33:107-122, 1998). Activation of both the classical and alternative pathways of complement are involved in the disease and both C4d and Bb are sensitive markers of moderate-to-severe lupus disease activity (Manzi, S., et al., Arthrit. Rheumat. 39:1178-1188, 1996). Activation of the alternative complement pathway accompanies disease flares in systemic lupus erythematosus during pregnancy (Buyon, J. P., et al., Arthritis Rheum. 35:55-61, 1992). In addition, the lectin pathway may contribute to disease development since autoantibodies against MBL have recently been identified in sera from SLE patients (Seelen, M. A., et al., Clin Exp. Immunol. 134:335-343, 2003).

Immune complex-mediated activation of complement through the classic pathway is believed to be one mechanism by which tissue injury occurs in SLE patients. However, hereditary deficiencies in complement components of the classic pathway increase the risk of lupus and lupus-like disease (Pickering, M. C., et al., Adv. Immunol. 76:227-324, 2000). SLE, or a related syndrome occurs in more than 80% of persons with complete deficiency of C1q, C1r/C1s, C4 or C3. This presents an apparent paradox in reconciling the harmful effects with the protective effects of complement in lupus.

An important activity of the classical pathway appears to be promotion of the removal of immune complexes from the circulation and tissues by the mononuclear phagocytic system (Kohler, P. F., et al., Am. J. Med. 56:406-11, 1974). In addition, complement has recently been found to have an important role in the removal and disposal of apoptotic bodies (Mevorarch, D., et al., J. Exp. Med. 188:2313-2320, 1998). Deficiency in classical pathway function may predispose subjects to the development of SLE by allowing a cycle to develop in which immune complexes or apoptotic cells accumulate in tissues, cause inflammation and the release of autoantigens, which in turn stimulate the production of autoantibodies and more immune complexes and thereby evoke an autoimmune response (Botto, M., et al., Nat. Genet. 19:56-59, 1998; Botto, M., Arthritis Res. 3:201-10, 2001). However, these “complete” deficiency states in classical pathway components are present in approximately one of 100 patients with SLE. Therefore, in the vast majority of SLE patients, complement deficiency in classical pathway components does not contribute to the disease etiology and complement activation may be an important mechanism contributing to SLE pathogenesis. The fact that rare individuals with permanent genetic deficiencies in classical pathway components frequently develop SLE at some point in their lives testifies to the redundancy of mechanisms capable of triggering the disease.

Results from animal models of SLE support the important role of complement activation in pathogenesis of the disease. Inhibiting the activation of C5 using a blocking anti-C5 MoAb decreased proteinuria and renal disease in NZB/NZW F1 mice, a mouse model of SLE (Wang Y., et al., Proc. Natl. Acad. Sci. USA 93:8563-8, 1996). Furthermore, treatment with anti-C5 MoAb of mice with severe combined immunodeficiency disease implanted with cells secreting anti-DNA antibodies results in improvement in the proteinuria and renal histologic picture with an associated benefit in survival compared to untreated controls (Ravirajan, C. T., et al., Rheumatology 43:442-7, 2004). The alternative pathway also has an important role in the autoimmune disease manifestations of SLE since backcrossing of factor B-deficient mice onto the MRL/lpr model of SLE revealed that the lack of factor B lessened the vasculitis, glomerular disease, C3 consumption and IgG3 RF levels typically found in this model without altering levels of other autoantibodies (Watanabe, H., et al., J. Immunol. 164:786-794, 2000). A humanized anti-C5 MoAb is under investigation as a potential treatment for SLE. This antibody prevents the cleavage of C5 to C5a and C5b. In Phase I clinical trials, no serious adverse effects were noted, and more human trials are under way to determine the efficacy in SLE (Strand, V., Lupus 10:216-221, 2001).

One aspect of the invention is thus directed to the prevention or treatment of inflammatory and non-inflammatory arthritides and other musculoskeletal disorders, including but not limited to osteoarthritis, rheumatoid arthritis, gout, neuropathic arthropathy, juvenile rheumatoid arthritis, psoriatic arthritis, ankylosing spondylitis or other spondyloarthropathies and crystalline arthropathies, or systemic lupus erythematosus (SLE), by administering a composition comprising a therapeutically effective amount of a MAp19 inhibitory agent in a pharmaceutical carrier to a subject suffering from such a disorder. The MAp19 inhibitory agent may be administered to the subject systemically, such as by intra-arterial, intravenous, intramuscular, subcutaneous, or other parenteral administration, or potentially by oral administration for nonpeptidergic agents. Alternatively, administration may be by local delivery, such as by intra-articular injection. The MAp19 inhibitory agent may be administered periodically over an extended period of time for treatment or control of a chronic condition, or may be by single or repeated administration in the period before, during, and/or following acute trauma or injury, including surgical procedures performed on the joint.

Renal Conditions

Activation of the complement system has been implicated in the pathogenesis of a wide variety of renal diseases, including: mesangioproliferative glomerulonephritis (IgA-nephropathy, Berger's disease) (Endo, M., et al., Clin. Nephrology 55:185-191, 2001), membranous glomerulonephritis (Kerjashki, D., Arch. B Cell Pathol. 58:253-71, 1990; Brenchley, P. E., et al., Kidney Int., 41:933-7, 1992; Salant, D. J., et al., Kidney Int. 35:976-84, 1989), membranoproliferative glomerulonephritis (mesangiocapillary glomerulonephritis) (Bartlow, B. G., et al., Kidney Int. 15:294-300, 1979; Meri, S., et al., J. Exp. Med. 175:939-50, 1992), acute postinfectious glomerulonephritis (poststreptococcal glomerulonephritis), cryoglobulinemic glomerulonephritis (Ohsawa, I., et al., Clin Immunol. 101:59-66, 2001), lupus nephritis (Gatenby, P. A., Autoimmunity 11:61-6, 1991), and Henoch-Schonlein purpura nephritis (Endo, M., et al., Am. J. Kidney Dis. 35:401-407, 2000). The involvement of complement in renal disease has been appreciated for several decades, but there is still a major discussion on its exact role in the onset, the development, and the resolution phase of renal disease. Under normal conditions, the contribution of complement is beneficial to the host, but inappropriate activation and deposition of complement may contribute to tissue damage.

There is substantial evidence that glomerulonephritis, inflammation of the glomeruli, is often initiated by deposition of immune complexes onto glomerular or tubular structures which then triggers complement activation, inflammation and tissue damage. Kahn and Sinniah demonstrated increased deposition of C5b-9 in tubular basement membranes in biopsies taken from patients with various forms of glomerulonephritis (Kahn, T. N., et al., Histopath. 26:351-6, 1995). In a study of patients with IgA nephrology (Alexopoulos, A., et al., Nephrol. Dial. Transplant 10:1166-1172, 1995), C5b-9 deposition in the tubular epithelial/basement membrane structures correlated with plasma creatinine levels. Another study of membranous nephropathy demonstrated a relationship between clinical outcome and urinary sC5b-9 levels (Kon, S. P., et al., Kidney Int. 48:1953-58, 1995). Elevated sC5b-9 levels were correlated positively with poor prognosis. Lehto et al. measured elevated levels of CD59, a complement regulatory factor that inhibits the membrane attack complex in plasma membranes, as well as C5b-9 in urine from patients with membranous glomerulonephritis (Lehto, T., et al., Kidney Int. 47:1403-11, 1995). Histopathological analysis of biopsy samples taken from these same patients demonstrated deposition of C3 and C9 proteins in the glomeruli, whereas expression of CD59 in these tissues was diminished compared to that of normal kidney tissue. These various studies suggest that ongoing complement-mediated glomerulonephritis results in urinary excretion of complement proteins that correlate with the degree of tissue damage and disease prognosis.

Inhibition of complement activation in various animal models of glomerulonephritis has also demonstrated the importance of complement activation in the etiology of the disease. In a model of membranoproliferative glomerulonephritis (MPGN), infusion of anti-Thy1 antiserum in C6-deficient rats (that cannot form C5b-9) resulted in 90% less glomerular cellular proliferation, 80% reduction in platelet and macrophage infiltration, diminished collagen type IV synthesis (a marker for mesangial matrix expansion), and 50% less proteinuria than in C6+normal rats (Brandt, J., et al., Kidney Int. 49:335-343, 1996). These results implicate C5b-9 as a major mediator of tissue damage by complement in this rat anti-thymocyte serum model. In another model of glomerulonephritis, infusion of graded dosages of rabbit anti-rat glomerular basement membrane produced a dose-dependent influx of polymorphonuclear leukocytes (PMN) that was attenuated by prior treatment with cobra venom factor (to consume complement) (Scandrett, A. L., et al., Am. J. Physiol. 268:F256-F265, 1995). Cobra venom factor-treated rats also showed diminished histopathology, decreased long-term proteinuria, and lower creatinine levels than control rats. Employing three models of GN in rats (anti-thymocyte serum, Con A anti-Con A, and passive Heymann nephritis), Couser et al. demonstrated the potential therapeutic efficacy of approaches to inhibit complement by using the recombinant sCR1 protein (Couser, W. G., et al., J. Am. Soc. Nephrol. 5:1888-94, 1995). Rats treated with sCR1 showed significantly diminished PMN, platelet and macrophage influx, decreased mesangiolysis, and proteinuria versus control rats. Further evidence for the importance of complement activation in glomerulonephritis has been provided by the use of an anti-C5 MoAb in the NZB/W F1 mouse model. The anti-C5 MoAb inhibits cleavage of C5, thus blocking generation of C5a and C5b-9. Continuous therapy with anti-C5 MoAb for 6 months resulted in significant amelioration of the course of glomerulonephritis. A humanized anti-C5 MoAb monoclonal antibody (5G1.1) that prevents the cleavage of human complement component C5 into its pro-inflammatory components is under development by Alexion Pharmaceuticals, Inc., New Haven, Conn., as a potential treatment for glomerulonephritis.

Direct evidence for a pathological role of complement in renal injury is provided by studies of patients with genetic deficiencies in specific complement components. A number of reports have documented an association of renal disease with deficiencies of complement regulatory factor H (Ault, B. H., Nephrol. 14:1045-1053, 2000; Levy, M., et al., Kidney Int. 30:949-56, 1986; Pickering, M. C., et al., Nat. Genet. 31:424-8, 2002). Factor H deficiency results in low plasma levels of factor B and C3 and in consumption of CSb-9. Both a typical membrane proliferative glomerulonnephritis (MPGN) and idiopathic hemolytic uremic syndrome (HUS) are associated with factor H deficiency. Factor H deficient pigs (Jansen, J. H., et al., Kidney Int. 53:331-49, 1998) and factor H knockout mice (Pickering, M. C., 2002) display MPGN-like symptoms, confirming the importance of factor H in complement regulation. Deficiencies of other complement components are associated with renal disease, secondary to the development of systemic lupus erythematosus (SLE) (Walport, M. J., Davies, et al., Ann. N.Y. Acad. Sci. 815:267-81, 1997). Deficiency for C1q, C4 and C2 predispose strongly to the development of SLE via mechanisms relating to defective clearance of immune complexes and apoptotic material. In many of these SLE patients lupus neuritis occurs, characterized by the deposition of immune complexes throughout the glomerulus.

Further evidence linking complement activation and renal disease has been provided by the identification in patients of autoantibodies directed against complement components, some of which have been directly related to renal disease (Trouw, L. A., et al., Mol. Immunol. 38:199-206, 2001). A number of these autoantibodies show such a high degree of correlation with renal disease that the term nephritic factor (NeF) was introduced to indicate this activity. In clinical studies, about 50% of the patients positive for nephritic factors developed MPGN (Spitzer, R. E., et al., Clin. Immunol. Immunopathol. 64:177-83, 1992). C3NeF is an autoantibody directed against the alternative pathway C3 convertase (C3bBb) and it stabilizes this convertase, thereby promoting alternative pathway activation (Daha, M. R., et al., J. Immunol. 116:1-7, 1976). Likewise, autoantibody with a specificity for the classical pathway C3 convertase (C4b2a), called C4NeF, stabilizes this convertase and thereby promotes classical pathway activation (Daha, M. R. et al., J. Immunol. 125:2051-2054, 1980; Halbwachs, L., et al., J. Clin. Invest. 65:1249-56, 1980). Anti-C1q autoantibodies have been described to be related to nephritis in SLE patients (Hovath, L., et al., Clin. Exp. Rheumatol. 19:667-72, 2001; Siegert, C., et al., J. Rheumatol. 18:230-34, 1991; Siegert, C., et al., Clin. Exp. Rheumatol. 10:19-23, 1992), and a rise in the titer of these anti-C1q autoantibodies was reported to predict a flare of nephritis (Coremans, I. E., et al., Am. J. Kidney Dis. 26:595-601, 1995). Immune deposits eluted from postmortem kidneys of SLE patients revealed the accumulation of these anti-C1q autoantibodies (Mannick, M. et al., Arthritis Rheumatol. 40:1504-11, 1997). All these facts point to a pathological role for these autoantibodies. However, not all patients with anti-C1q autoantibodies develop renal disease and also some healthy individuals have low titer anti-C1q autoantibodies (Siegert, C. E., et al., Clin. Immunol. Immunopathol. 67:204-9, 1993).

In addition to the alternative and classical pathways of complement activation, the lectin pathway may also have an important pathological role in renal disease. Elevated levels of MBL, MBL-associated serine protease and complement activation products have been detected by immunohistochemical techniques on renal biopsy material obtained from patients diagnosed with several different renal diseases, including Henoch-Schonlein purpura nephritis (Endo, M., et al., Am. J. Kidney Dis. 35:401-407, 2000), cryoglobulinemic glomerulonephritis (Ohsawa, I., et al., Clin Immunol. 101:59-66, 2001) and IgA neuropathy (Endo, M., et al., Clin. Nephrology 55:185-191, 2001). Therefore, despite the fact that an association between complement and renal diseases has been known for several decades, data on how complement exactly influences these renal diseases is far from complete.

One aspect of the invention is thus directed to the treatment of renal conditions including but not limited to mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis or IgA nephropathy, by administering a composition comprising a therapeutically effective amount of a MAp19 inhibitory agent in a pharmaceutical carrier to a subject suffering from such a disorder. The MAp19 inhibitory agent may be administered to the subject systemically, such as by intra-arterial, intravenous, intramuscular, subcutaneous, or other parenteral administration, or potentially by oral administration for nonpeptidergic agents. The MAp19 inhibitory agent may be administered periodically over an extended period of time for treatment or control of a chronic condition, or may be by single or repeated administration in the period before, during or following acute trauma or injury.

Skin Disorders

Psoriasis is a chronic, debilitating skin condition that affects millions of people and is attributed to both genetic and environmental factors. Topical agents as well as UVB and PUVA phototherapy are generally considered to be the first-line treatment for psoriasis. However, for generalized or more extensive disease, systemic therapy is indicated as a primary treatment or, in some cases, to potentiate UVB and PUVA therapy.

The underlying etiology of various skin diseases such as psoriasis support a role for immune and proinflammatory processes including the involvement of the complement system. Moreover, the role of the complement system has been established as an important nonspecific skin defense mechanism. Its activation leads to the generation of products that not only help to maintain normal host defenses, but also mediate inflammation and tissue injury. Proinflammatory products of complement include large fragments of C3 with opsonic and cell-stimulatory activities (C3b and C3bi), low molecular weight anaphylatoxins (C3a, C4a, and C5a), and membrane attack complexes. Among them, C5a or its degradation product C5a des Arg, seems to be the most important mediator because it exerts a potent chemotactic effect on inflammatory cells. Intradermal administration of C5a anaphylatoxin induces skin changes quite similar to those observed in cutaneous hypersensitivity vasculitis that occurs through immune complex-mediated complement activation. Complement activation is involved in the pathogenesis of the inflammatory changes in autoimmune bullous dermatoses. Complement activation by pemphigus antibody in the epidermis seems to be responsible for the development of characteristic inflammatory changes termed eosinophilic spongiosis. In bullous pemphigoid (BP), interaction of basement membrane zone antigen and BP antibody leads to complement activation that seems to be related to leukocytes lining the dermoepidermal junction. Resultant anaphylatoxins not only activate the infiltrating leukocytes but also induce mast cell degranulation, which facilitates dermoepidermal separation and eosinophil infiltration. Similarly, complement activation seems to play a more direct role in the dermoepidermal separation noted in epidermolysis bullosa acquisita and herpes gestationis.

Evidence for the involvement of complement in psoriasis comes from recent experimental findings described in the literature related to the pathophysiological mechanisms for the inflammatory changes in psoriasis and related diseases. A growing body of evidence has indicated that T-cell-mediated immunity plays an important role in the triggering and maintenance of psoriatic lesions. It has been revealed that lymphokines produced by activated T-cells in psoriatic lesions have a strong influence on the proliferation of the epidermis. Characteristic neutrophil accumulation under the stratum corneum can be observed in the highly inflamed areas of psoriatic lesions. Neutrophils are chemotactically attracted and activated there by synergistic action of chemokines, IL-8 and Gro-alpha released by stimulated keratinocytes, and particularly by C5a/C5a des-arg produced via the alternative complement pathway activation (Terui, T., Tahoku J. Exp. Med. 190:239-248, 2000; Terui, T., Exp. Dermatol. 9:1-10, 2000).

Psoriatic scale extracts contain a unique chemotactic peptide fraction that is likely to be involved in the induction of rhythmic transepidermal leukocyte chemotaxis. Recent studies have identified the presence of two unrelated chemotactic peptides in this fraction, i.e., C5a/C5a des Arg and interleukin 8 (IL-8) and its related cytokines. To investigate their relative contribution to the transepidermal leukocyte migration as well as their interrelationship in psoriatic lesions, concentrations of immunoreactive C5a/C5a desArg and IL-8 in psoriatic lesional scale extracts and those from related sterile pustular dermatoses were quantified. It was found that the concentrations of C5a/C5a desArg and IL-8 were more significantly increased in the horny-tissue extracts from lesional skin than in those from non-inflammatory orthokeratotic skin. The increase of C5a/C5a desArg concentration was specific to the lesional scale extracts. Based on these results, it appears that C5a/C5a desArg is generated only in the inflammatory lesional skin under specific circumstances that preferentially favor complement activation. This provides a rationale for the use of an inhibitor of complement activation to ameliorate psoriatic lesions.

While the classical pathway of the complement system has been shown to be activated in psoriasis, there are fewer reports on the involvement of the alternative pathway in the inflammatory reactions in psoriasis. Within the conventional view of complement activation pathways, complement fragments C4d and Bb are released at the time of the classical and alternative pathway activation, respectively. The presence of the C4d or Bb fragment, therefore, denotes a complement activation that proceeds through the classical and/or alternative pathway. One study measured the levels of C4d and Bb in psoriatic scale extracts using enzyme immunoassay techniques. The scales of these dermatoses contained higher levels of C4d and Bb detectable by enzyme immunoassay than those in the stratum corneum of noninflammatory skin (Takematsu, H., et al., Dermatologica 181:289-292, 1990). These results suggest that the alternative pathway is activated in addition to the classical pathway of complement in psoriatic lesional skin.

Additional evidence for the involvement of complement in psoriasis and atopic dermatitis has been obtained by measuring normal complement components and activation products in the peripheral blood of 35 patients with atopic dermatitis (AD) and 24 patients with psoriasis at a mild to intermediate stage. Levels of C3, C4 and C1 inactivator (C1 INA) were determined in serum by radial immunodiffusion, whereas C3a and C5a levels were measured by radioimmunoassay. In comparison to healthy non-atopic controls, the levels of C3, C4 and C1 INA were found to be significantly increased in both diseases. In AD, there was a tendency towards increased C3a levels, whereas in psoriasis, C3a levels were significantly increased. The results indicate that, in both AD and psoriasis, the complement system participates in the inflammatory process (Ohkonohchi, K., et al., Dermatologica 179:30-34, 1989).

Complement activation in psoriatic lesional skin also results in the deposition of terminal complement complexes within the epidermis as defined by measuring levels of SC5b-9 in the plasma and horny tissues of psoriatic patients. The levels of SC5b-9 in psoriatic plasma have been found to be significantly higher than those of controls or those of patients with atopic dermatitis. Studies of total protein extracts from lesional skin have shown that, while no SC5b-9 can be detected in the noninflammatory horny tissues, there were high levels of SC5b-9 in lesional horny tissues of psoriasis. By immunofluorescence using a monoclonal antibody to the C5b-9 neoantigen, deposition of C5b-9 has been observed only in the stratum corneum of psoriatic skin. In summary, in psoriatic lesional skin, the complement system is activated and complement activation proceeds all the way to the terminal step, generating membrane attack complex.

New biologic drugs that selectively target the immune system have recently become available for treating psoriasis. Four biologic drugs that are either currently FDA approved or in Phase 3 studies are: alefacept (Amevive®) and efalizuMoAb (Raptiva®), which are T-cell modulators; etanercept (Enbrel®), a soluble TNF-receptor; and inflixiMoAb (Remicade®), an anti-TNF monoclonal antibody. Raptiva is an immune response modifier, wherein the targeted mechanism of action is a blockade of the interaction between LFA-1 on lymphocytes and ICAM-1 on antigen-presenting cells and on vascular endothelial cells. Binding of CD11a by Raptiva results in saturation of available CD11a binding sites on lymphocytes and down-modulation of cell surface CD11a expression on lymphocytes. This mechanism of action inhibits T-cell activation, cell trafficking to the dermis and epidermis and T-cell reactivation. Thus, a plurality of scientific evidence indicates a role for complement in inflammatory disease states of the skin and recent pharmaceutical approaches have targeted the immune system or specific inflammatory processes. None, however, have identified MAp19 as a targeted approach. Based on the inventors' new understanding of the role of MAp19 in complement activation, the inventors believe MAp19 to be an effective target for the treatment of psoriasis and other skin disorders.

One aspect of the invention is thus directed to the treatment of psoriasis, autoimmune bullous dermatoses, eosinophilic spongiosis, bullous pemphigoid, epidermolysis bullosa acquisita, atopic dermatitis, herpes gestationis and other skin disorders, and for the treatment of thermal and chemical burns including capillary leakage caused thereby, by administering a composition comprising a therapeutically effective amount of a MAp19 inhibitory agent in a pharmaceutical carrier to a subject suffering from such a skin disorder. The MAp19 inhibitory agent may be administered to the subject topically, by application of a spray, lotion, gel, paste, salve or irrigation solution containing the MAp19 inhibitory agent, or systemically such as by intra-arterial, intravenous, intramuscular, subcutaneous or other parenteral administration, or potentially by oral administration for nonpeptidergic inhibitors. Treatment may involve a single administration or repeated applications or dosings for an acute condition, or by periodic applications or dosings for control of a chronic condition.

Transplantation

Activation of the complement system significantly contributes to the inflammatory reaction after solid organ transplantation. In allotransplantation, the complement system may be activated by ischemia/reperfusion and, possibly, by antibodies directed against the graft (Baldwin, W. M., et al., Springer Seminol. Immunopathol. 25:181-197, 2003). In xenotransplantation from nonprimates to primates, the major activators for complement are preexisting antibodies. Studies in animal models have shown that the use of complement inhibitors may significantly prolong graft survival (see below). Thus, there is an established role of the complement system in organ injury after organ transplantation, and therefore the inventors believe that the use of complement inhibitors directed to MAp19 may prevent damage to the graft after allo- or xenotransplantation.

Innate immune mechanisms, particularly complement, play a greater role in inflammatory and immune responses against the graft than has been previously recognized. For example, alternative complement pathway activation appears to mediate renal ischemia/reperfusion injury, and proximal tubular cells may be both the source and the site of attack of complement components in this setting. Locally produced complement in the kidney also plays a role in the development of both cellular and antibody-mediated immune responses against the graft.

C4d is the degradation product of the activated complement factor C4, a component of the classical and lectin-dependent pathways. C4d staining has emerged as a useful marker of humoral rejection both in the acute and in the chronic setting and led to renewed interest in the significance of anti-donor antibody formation. The association between C4d and morphological signs of acute cellular rejection is statistically significant. C4d is found in 24-43% of Type I episodes, in 45% of type II rejection and 50% of type III rejection (Nickeleit, V., et al., J. Am. Soc. Nephrol. 13:242-251, 2002; Nickeleit, V., et al., Nephrol. Dial. Transplant 18:2232-2239, 2003). A number of therapies are in development that inhibit complement or reduce local synthesis as a means to achieve an improved clinical outcome following transplantation.

Activation of the complement cascade occurs as a result of a number of processes during transplantation. Present therapy, although effective in limiting cellular rejection, does not fully deal with all the barriers faced. These include humoral rejection and chronic allograft nephropathy or dysfunction. Although the overall response to the transplanted organ is a result of a number of effector mechanisms on the part of the host, complement may play a key role in some of these. In the setting of renal transplantation, local synthesis of complement by proximal tubular cells appears of particular importance.

The availability of specific inhibitors of complement may provide the opportunity for an improved clinical outcome following organ transplantation. Inhibitors that act by a mechanism that blocks complement attack may be particularly useful, because they hold the promise of increased efficacy and avoidance of systemic complement depletion in an already immuno-compromised recipient.

Complement also plays a critical role in xenograft rejection. Therefore, effective complement inhibitors are of great interest as potential therapeutic agents. In pig-to-primate organ transplantation, hyperacute rejection (HAR) results from antibody deposition and complement activation. Multiple strategies and targets have been tested to prevent hyperacute xenograft rejection in the pig-to-primate combination. These approaches have been accomplished by removal of natural antibodies, complement depletion with cobra venom factor, or prevention of C3 activation with the soluble complement inhibitor sCR1. In addition, complement activation blocker-2 (CAB-2), a recombinant soluble chimeric protein derived from human decay accelerating factor (DAF) and membrane cofactor protein, inhibits C3 and C5 convertases of both classical and alternative pathways. CAB-2 reduces complement-mediated tissue injury of a pig heart perfused ex vivo with human blood. A study of the efficacy of CAB-2 when a pig heart was transplanted heterotopically into rhesus monkeys receiving no immunosuppression showed that graft survival was markedly prolonged in monkeys that received CAB-2 (Salerno, C. T., et al., Xenotransplantation 9:125-134, 2002). CAB-2 markedly inhibited complement activation, as shown by a strong reduction in generation of C3a and SC5b-9. At graft rejection, tissue deposition of iC3b, C4 and C9 was similar or slightly reduced from controls, and deposition of IgG, IgM, C1q and fibrin did not change. Thus, this approach for complement inhibition abrogated hyperacute rejection of pig hearts transplanted into rhesus monkeys. These studies demonstrate the beneficial effects of complement inhibition on survival and the inventors believe that MASP-2 inhibition may also be useful in xenotransplantation.

Another approach has focused on determining if anti-complement 5 (C5) monoclonal antibodies could prevent hyperacute rejection (HAR) in a rat-to-presensitized mouse heart transplantation model and whether these MoAb, combined with cyclosporine and cyclophosphamide, could achieve long-term graft survival. It was found that anti-C5 MoAb prevents HAR (Wang, H., et al., Transplantation 68:1643-1651, 1999). The inventors thus believe that other targets in the complement cascade, such as MAp19, may also be valuable for preventing HAR and acute vascular rejection in future clinical xenotransplantation.

While the pivotal role of complement in hyperacute rejection seen in xenografts is well established, a subtler role in allogeneic transplantation is emerging. A link between complement and the acquired immune response has long been known, with the finding that complement-depleted animals mounted subnormal antibody responses following antigenic stimulation. Opsonization of antigen with the complement split product C3d has been shown to greatly increase the effectiveness of antigen presentation to B cells, and has been shown to act via engagement of complement receptor type 2 on certain B cells. This work has been extended to the transplantation setting in a skin graft model in mice, where C3- and C4-deficient mice had a marked defect in allo-antibody production, due to failure of class switching to high-affinity IgG. The importance of these mechanisms in renal transplantation is increased due to the significance of anti-donor antibodies and humoral rejection.

Previous work has already demonstrated upregulation of C3 synthesis by proximal tubular cells during allograft rejection following renal transplantation. The role of locally synthesized complement has been examined in a mouse renal transplantation model. Grafts from C3-negative donors transplanted into C3-sufficient recipients demonstrated prolonged survival (>100 days) as compared with control grafts from C3-positive donors, which were rejected within 14 days. Furthermore, the anti-donor T-cell proliferative response in recipients of C3-negative grafts was markedly reduced as compared with that of controls, indicating an effect of locally synthesized C3 on T-cell priming.

These observations suggest the possibility that exposure of donor antigen to T-cells first occurs in the graft and that locally synthesized complement enhances antigen presentation, either by opsonization of donor antigen or by providing additional signals to both antigen-presenting cells and T-cells. In the setting of renal transplantation, tubular cells that produce complement also demonstrate complement deposition on their cell surface.

One aspect of the invention is thus directed to the prevention or treatment of inflammatory reaction resulting from tissue or solid organ transplantation by administering a composition comprising a therapeutically effective amount of a MAp19 inhibitory agent in a pharmaceutical carrier to the transplant recipient, including subjects that have received allotransplantation or xenotransplantation of whole organs (e.g., kidney, heart, liver, pancreas, lung, cornea, etc.) or grafts (e.g., valves, tendons, bone marrow, etc.). The MAp19 inhibitory agent may be administered to the subject by intra-arterial, intravenous, intramuscular, subcutaneous or other parenteral administration, or potentially by oral administration for non-peptidergic inhibitors. Administration may occur during the acute period following transplantation and/or as long-term posttransplantation therapy. Additionally or in lieu of posttransplant administration, the subject may be treated with the MAp19 inhibitory agent prior to transplantation and/or during the transplant procedure, and/or by pretreating the organ or tissue to be transplanted with the MAp19 inhibitory agent. Pretreatment of the organ or tissue may entail applying a solution, gel or paste containing the MAp19 inhibitory agent to the surface of the organ or tissue by spraying or irrigating the surface, or the organ or tissue may be soaked in a solution containing the MAp19 inhibitor.

Central and Peripheral Nervous System Disorders and Injuries

Activation of the complement system has been implicated in the pathogenesis of a variety of central nervous system (CNS) or peripheral nervous system (PNS) diseases or injuries, including but not limited to multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, degenerative discs, Parkinson's disease (PD), and Alzheimer's disease (AD). The initial determination that complement proteins are synthesized in CNS cells including neurons, astrocytes and microglia, as well as the realization that anaphylatoxins generated in the CNS following complement activation can alter neuronal function, has opened up the potential role of complement in CNS disorders (Morgan, B. P., et al., Immunology Today 17:10: 461-466, 1996). It has now been shown that C3a receptors and C5a receptors are found on neurons and show widespread distribution in distinct portions of the sensory, motor and limbic brain systems (Barum, S. R., Immunologic Research 26:7-13, 2002). Moreover, the anaphylatoxins C5a and C3a have been shown to alter eating and drinking behavior in rodents and can induce calcium signaling in microglia and neurons. These findings raise possibilities regarding the therapeutic utility of inhibiting complement activation in a variety of CNS inflammatory diseases including cerebral trauma, demyelination, meningitis, stroke, and Alzheimer's disease.

Brain trauma or hemorrhage is a common clinical problem, and complement activation may occur and exacerbate resulting inflammation and edema. The effects of complement inhibition have been studied in a model of brain trauma in rats (Kaczorowski et al., J. Cereb. Blood Flow Metab. 15:860-864, 1995). Administration of sCR1 immediately prior to brain injury markedly inhibited neutrophil infiltration into the injured area, indicating complement was important for recruitment of phagocytic cells. Likewise, complement activation in patients following cerebral hemorrhage is clearly implicated by the presence of high levels of multiple complement activation products in both plasma and cerebrospinal fluid (CSF). Complement activation and increased staining of C5b-9 complexes have been demonstrated in sequestered lumbar disc tissue and could suggest a role in disc herniation tissue-induced sciatica (Gronblad, M., et al., Spine 28(2):114-118, 2003).

MS is characterized by a progressive loss of myelin ensheathing and insulating axons within the CNS. Although the initial cause is unknown, there is abundant evidence implicating the immune system (Prineas, J. W., et al., Lab Invest. 38:409-421, 1978; Ryberg, B., J. Neurol. Sci. 54:239-261, 1982). There is also clear evidence that complement plays a prominent role in the pathophysiology of CNS or PNS demyelinating diseases including MS, Guillain-Barre syndrome and Miller-Fisher syndrome (Gasque, P., et al., Immunopharmacology 49:171-186, 2000; Barnum, S. R., in Bondy, S., et al. (eds.), Inflammatory events in neurodegeneration, Prominent Press 139-156, 2001). Complement contributes to tissue destruction, inflammation, clearance of myelin debris and even remyelination of axons. Despite clear evidence of complement involvement, the identification of complement therapeutic targets is only now being evaluated in experimental allergic encephalomyelitis (EAE), an animal model of multiple sclerosis. Studies have established that EAE mice deficient in C3 or factor B showed attenuated demyelination as compared to EAE control mice (Barnum, Immunologic Res. 26:7-13, 2002). EAE mouse studies using a soluble form of a complement inhibitor coined “sCrry” and C3−/− and factor B−/− demonstrated that complement contributes to the development and progression of the disease model at several levels. In addition, the marked reduction in EAE severity in factor B−/− mice provides further evidence for the role of the alternative pathway of complement in EAE (Nataf et al., J. Immunol. 165:5867-5873, 2000).

MG is a disease of the neuromuscular junction with a loss of acetylcholine receptors and destruction of the end plate. sCR1 is very effective in an animal model of MG, further indicating the role of complement in the disease (Piddelesden et al., J. Neuroimmunol. 1997).

The histological hallmarks of AD, a neurodegenerative disease, are senile plaques and neurofibrillary tangles (McGeer et al., Res. Immunol. 143:621-630, 1992). These pathological markers also stain strongly for components of the complement system. Evidence points to a local neuroinflammatory state that results in neuronal death and cognitive dysfunction. Senile plaques contain abnormal amyloid-β-peptide (Aβ), a peptide derived from amyloid precursor protein. Aβ has been shown to bind C1 and can trigger complement activation (Rogers et al., Res. Immunol. 143:624-630, 1992). In addition, a prominent feature of AD is the association of activated proteins of the classical complement pathway from C1q to C5b-9, which have been found highly localized in the neuritic plaques (Shen, Y., et al., Brain Research 769:391-395, 1997; Shen, Y., et al., Neurosci. Letters 305(3):165-168, 2001). Thus, Aβ not only initiates the classical pathway, but a resulting continual inflammatory state may contribute to the neuronal cell death. Moreover, the fact that complement activation in AD has progressed to the terminal C5b-9 phase indicates that the regulatory mechanisms of the complement system have been unable to halt the complement activation process.

Several inhibitors of the complement pathway have been proposed as potential therapeutic approaches for AD, including proteoglycan as inhibitors of C1Q binding, Nafamstat as an inhibitor of C3 convertase, and C5 activation blockers or inhibitors of C5a receptors (Shen, Y., et al., Progress in Neurobiology, 70:463-472, 2003). The role of MASP-2 as an initiation step in the innate complement pathway, as well as for alternative pathway activation, provides a potential new therapeutic approach and is supported by the wealth of data suggesting complement pathway involvement in AD.

In damaged regions in the brains of PD patients, as in other CNS degenerative diseases, there is evidence of inflammation characterized by glial reaction (especially microglia), as well as increased expression of HLA-DR antigens, cytokines, and components of complement. These observations suggest that immune system mechanisms are involved in the pathogenesis of neuronal damage in PD. The cellular mechanisms of primary injury in PD have not been clarified, however, but it is likely that mitochondrial mutations, oxidative stress and apoptosis play a role. Furthermore, inflammation initiated by neuronal damage in the striatum and the substantial nigra in PD may aggravate the course of the disease. These observations suggest that treatment with complement inhibitory drugs may act to slow progression of PD (Czlonkowska, A., et al., Med. Sci. Monit. 8:165-177, 2002).

One aspect of the invention is thus directed to the treatment of peripheral nervous system (PNS) and/or central nervous system (CNS) disorders or injuries by treating a subject suffering from such a disorder or injury with a composition comprising a therapeutically effective amount of a MAp19 inhibitory agent in a pharmaceutical carrier. CNS and PNS disorders and injuries that may be treated in accordance with the present invention are believed to include but are not limited to multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, Parkinson's disease (PD), Alzheimer's disease (AD), Miller-Fisher syndrome, cerebral trauma and/or hemorrhage, demyelination and, possibly, meningitis.

For treatment of CNS conditions and cerebral trauma, the MAp19 inhibitory agent may be administered to the subject by intrathecal, intracranial, intraventricular, intra-arterial, intravenous, intramuscular, subcutaneous, or other parenteral administration, and potentially orally for non-peptidergic inhibitors. PNS conditions and cerebral trauma may be treated by these systemic route of administration or alternately by local administration to the site of dysfunction or trauma. Administration of the MAp19 inhibitory compositions of the present invention may be repeated periodically as determined by a physician until effective relief or control of the symptoms is achieved.

Blood Disorders

Sepsis is caused by an overwhelming reaction of the patient to invading microorganisms. A major function of the complement system is to orchestrate the inflammatory response to invading bacteria and other pathogens. Consistent with this physiological role, complement activation has been shown in numerous studies to have a major role in the pathogenesis of sepsis (Bone, R. C., Annals Internal. Med. 115:457-469, 1991). The definition of the clinical manifestations of sepsis is ever evolving. Sepsis is usually defined as the systemic host response to an infection. However, on many occasions, no clinical evidence for infection (e.g., positive bacterial blood cultures) is found in patients with septic symptoms. This discrepancy was first taken into account at a Consensus Conference in 1992 when the term “systemic inflammatory response syndrome” (SIRS) was established, and for which no definable presence of bacterial infection was required (Bone, R. C., et al., Crit. Care. Med. 20:724-726, 1992). There is now general agreement that sepsis and SIRS are accompanied by the inability to regulate the inflammatory response. For the purposes of this brief review, we will consider the clinical definition of sepsis to also include severe sepsis, septic shock, and SIRS.

The predominant source of infection in septic patients before the late 1980s was Gram-negative bacteria. Lipopolysaccharide (LPS), the main component of the Gram-negative bacterial cell wall, was known to stimulate release of inflammatory mediators from various cell types and induce acute infectious symptoms when injected into animals (Haeney, M. R., et al., Antimicrobial Chemotherapy 41(Suppl. A):41-6, 1998). Interestingly, the spectrum of responsible microorganisms appears to have shifted from predominantly Gram-negative bacteria in the late 1970s and 1980s to predominantly Gram-positive bacteria at present, for reasons that are currently unclear (Martin, G. S., et al., N. Eng. J. Med. 348:1546-54, 2003).

Many studies have shown the importance of complement activation in mediating inflammation and contributing to the features of shock, particularly septic and hemorrhagic shock. Both Gram-negative and Gram-positive organisms commonly precipitate septic shock. LPS is a potent activator of complement, predominantly via the alternative pathway, although classical pathway activation mediated by antibodies also occurs (Fearon, D. T., et al., N. Engl. J. Med. 292:937-400, 1975). The major components of the Gram-positive cell wall are peptidoglycan and lipoteichoic acid, and both components are potent activators of the alternative complement pathway, although in the presence of specific antibodies they can also activate the classical complement pathway (Joiner, K. A., et al., Ann. Rev. Immunol. 2:461-2, 1984).

The complement system was initially implicated in the pathogenesis of sepsis when it was noted by researchers that anaphylatoxins C3a and C5a mediate a variety of inflammatory reactions that might also occur during sepsis. These anaphylatoxins evoke vasodilation and an increase in microvascular permeability, events that play a central role in septic shock (Schumacher, W. A., et al., Agents Actions 34:345-349, 1991). In addition, the anaphylatoxins induce bronchospasm, histamine release from mast cells, and aggregation of platelets. Moreover, they exert numerous effects on granulocytes, such as chemotaxis, aggregation, adhesion, release of lysosomal enzymes, generation of toxic super oxide anion and formation of leukotrienes (Shin, H. S., et al., Science 162:361-363, 1968; Vogt, W., Complement 3:177-86, 1986). These biologic effects are thought to play a role in development of complications of sepsis such as shock or acute respiratory distress syndrome (ARDS) (Hammerschmidt, D. E., et al., Lancet 1:947-949, 1980; Slotman, G. T., et al., Surgery 99:744-50, 1986). Furthermore, elevated levels of the anaphylatoxin C3a is associated with a fatal outcome in sepsis (Hack, C. E., et al., Am. J. Med. 86:20-26, 1989). In some animal models of shock, certain complement-deficient strains (e.g., C5-deficient ones) are more resistant to the effects of LPS infusions (Hseuh, W., et al., Immunol. 70:309-14, 1990).

Blockade of C5a generation with antibodies during the onset of sepsis in rodents has been shown to greatly improve survival (Czermak, B. J., et al., Nat. Med. 5:788-792, 1999). Similar findings were made when the C5a receptor (C5aR) was blocked, either with antibodies or with a small molecular inhibitor (Huber-Lang, M. S., et al., FASEB J. 16:1567-74, 2002; Riedemann, N. C., et al., J. Clin. Invest. 110:101-8, 2002). Earlier experimental studies in monkeys have suggested that antibody blockade of C5a attenuated E. coli-induced septic shock and adult respiratory distress syndrome (Hangen, D. H., et al., J. Surg. Res. 46:195-9, 1989; Stevens, J. H., et al., J. Clin. Invest. 77:1812-16, 1986). In humans with sepsis, C5a was elevated and associated with significantly reduced survival rates together with multiorgan failure, when compared with that in less severely septic patients and survivors (Nakae, H., et al., Res. Commun. Chem. Pathol. Pharmacol. 84:189-95, 1994; Nakae, et al., Surg. Today 26:225-29, 1996; Bengtson, A., et al., Arch. Surg. 123:645-649, 1988). The mechanisms by which C5a exerts its harmful effects during sepsis are yet to be investigated in greater detail, but recent data suggest the generation of C5a during sepsis significantly compromises innate immune functions of blood neutrophils (Huber-Lang, M. S., et al., J. Immunol. 169:3223-31, 2002), their ability to express a respiratory burst, and their ability to generate cytokines (Riedemann, N. C., et al., Immunity 19:193-202, 2003). In addition, C5a generation during sepsis appears to have procoagulant effects (Laudes, I. J., et al., Am. J. Pathol. 160:1867-75, 2002). The complement-modulating protein CI INH has also shown efficacy in animal models of sepsis and ARDS (Dickneite, G., Behring Ins. Mitt. 93:299-305, 1993).

The lectin pathway may also have a role in pathogenesis of sepsis. MBL has been shown to bind to a range of clinically important microorganisms including both Gram-negative and Gram-positive bacteria, and to activate the lectin pathway (Neth, O., et al., Infect. Immun. 68:688, 2000). Lipoteichoic acid (LTA) is increasingly regarded as the Gram-positive counterpart of LPS. It is a potent immunostimulant that induces cytokine release from mononuclear phagocytes and whole blood (Morath, S., et al., J. Exp. Med. 195:1635, 2002; Morath, S. et al., Infect. Immun. 70:938, 2002). Recently it was demonstrated that L-ficolin specifically binds to LTA isolated from numerous Gram-positive bacteria species, including Staphylococcus aureus, and activates the lectin pathway (Lynch, N. J., et al., J. Immunol. 172:1198-202, 2004). MBL also has been shown to bind to LTA from Enterococcus spp in which the polyglycerophosphate chain is substituted with glycosyl groups), but not to LTA from nine other species including S. aureus (Polotsky, V. Y., et al., Infect. Immun. 64:380, 1996).

An aspect of the invention thus provides a method for treating sepsis or a condition resulting from sepsis by administering a composition comprising a therapeutically effective amount of a MAp19 inhibitory agent in a pharmaceutical carrier to a subject suffering from sepsis or a condition resulting from sepsis including, without limitation, severe sepsis, septic shock, acute respiratory distress syndrome resulting from sepsis, and systemic inflammatory response syndrome. Related methods are provided for the treatment of other blood disorders, including hemorrhagic shock, hemolytic anemia, autoimmune thrombotic thrombocytopenic purpura (TTP), hemolytic uremic syndrome (HUS) or other marrow/blood destructive conditions, by administering a composition comprising a therapeutically effective amount of a MAp19 inhibitory agent in a pharmaceutical carrier to a subject suffering from such a condition. The MAp19 inhibitory agent is administered to the subject systemically, such as by intra-arterial, intravenous, intramuscular, inhalational (particularly in the case of ARDS), subcutaneous or other parenteral administration, or potentially by oral administration for non-peptidergic agents. The MAp19 inhibitory agent composition may be combined with one or more additional therapeutic agents to combat the sequelae of sepsis and/or shock. For advanced sepsis or shock or a distress condition resulting therefrom, the MAp19 inhibitory composition may suitably be administered in a fast-acting dosage form, such as by intravenous or intra-arterial delivery of a bolus of a solution containing the MAp19 inhibitory agent composition. Repeated administration may be carried out as determined by a physician until the condition has been resolved.

Urogenital Conditions

The complement system has been implicated in several distinct urogenital disorders including painful bladder disease, sensory bladder disease, chronic abacterial cystitis and interstitial cystitis (Holm-Bentzen, M., et al., J. Urol. 138:503-507, 1987), infertility (Cruz, et al., Biol. Reprod. 54:1217-1228, 1996), pregnancy (Xu, C., et al., Science 287:498-507, 2000), fetomaternal tolerance (Xu, C., et al., Science 287:498-507, 2000), and pre-eclampsia (Haeger, M., Int. J. Gynecol. Obstet. 43:113-127, 1993).

Painful bladder disease, sensory bladder disease, chronic abacterial cystitis and interstitial cystitis are ill-defined conditions of unknown etiology and pathogenesis, and, therefore, they are without any rational therapy. Pathogenetic theories concerning defects in the epithelium and/or mucous surface coating of the bladder, and theories concerning immunological disturbances, predominate (Holm-Bentzen, M., et al., J. Urol. 138:503-507, 1987). Patients with interstitial cystitis were reported to have been tested for immunoglobulins (IgA, G, M), complement components (C1q, C3, C4) and for C1-esterase inhibitor. There was a highly significant depletion of the serum levels of complement component C4 (p less than 0.001) and immunoglobulin G was markedly elevated (p less than 0.001). This study suggests classical pathway activation of the complement system, and supports the possibility that a chronic local immunological process is involved in the pathogenesis of the disease (Mattila, J., et al., Eur. Urol. 9:350-352, 1983). Moreover, following binding of autoantibodies to antigens in bladder mucosa, activation of complement could be involved in the production of tissue injury and in the chronic self-perpetuating inflammation typical of this disease (Helin, H., et al., Clin. Immunol. Immunopathol. 43:88-96, 1987).

In addition to the role of complement in urogenital inflammatory diseases, reproductive functions may be impacted by the local regulation of the complement pathway. Naturally occurring complement inhibitors have evolved to provide host cells with the protection they need to control the body's complement system. Crry, a naturally-occurring rodent complement inhibitor that is structurally similar to the human complement inhibitors, MCP and DAF, has been investigated to delineate the regulatory control of complement in fetal development. Interesting, attempts to generate Crry−/− mice were unsuccessful. Instead, it was discovered that homozygous Crry−/− mice died in utero. Crry−/− embryos survived until about 10 days post coitus, and survival rapidly declined with death resulting from developmental arrest. There was also a marked invasion of inflammatory cells into the placental tissue of Crry−/− embryos. In contrast, Crry+/+ embryos appeared to have C3 deposited on the placenta. This suggests that complement activation had occurred at the placenta level, and in the absence of complement regulation, the embryos died. Confirming studies investigated the introduction of the Crry mutation onto a C3 deficient background. This rescue strategy was successful. Together, these data illustrate that the fetomaternal complement interface must be regulated. Subtle alterations in complement regulation within the placenta might contribute to placental dysfunction and miscarriage (Xu, C., et al., Science 287:498-507, 2000).

Pre-eclampsia is a pregnancy-induced hypertensive disorder in which complement system activation has been implicated but remains controversial (Haeger, M., Int. J. Gynecol. Obstet. 43:113-127, 1993). Complement activation in systemic circulation is closely related to established disease in pre-eclampsia, but no elevations were seen prior to the presence of clinical symptoms and, therefore, complement components cannot be used as predictors of pre-eclampsia (Haeger et al., Obstet. Gynecol. 78:46, 1991). However, increased complement activation at the local environment of the placenta bed might overcome local control mechanisms, resulting in raised levels of anaphylatoxins and C5b-9 (Haeger et al., Obstet. Gynecol. 73:551, 1989).

One proposed mechanism of infertility related to antisperm antibodies (ASA) is through the role of complement activation in the genital tract. Generation of C3b and iC3b opsonin, which can potentiate the binding of sperm by phagocytic cells via their complement receptors as well as formation of the terminal C5b-9 complex on the sperm surface, thereby reducing sperm motility, are potential causes associated with reduced fertility. Elevated C5b-9 levels have also been demonstrated in ovarian follicular fluid of infertile women (D'Cruz, O. J., et al., J. Immunol. 144:3841-3848, 1990). Other studies have shown impairment in sperm migration, and reduced sperm/egg interactions, which may be complement associated (D'Cruz, O. J., et al., J. Immunol. 146:611-620, 1991; Alexander, N. J., Fertil. Steril. 41:433-439, 1984). Finally, studies with sCR1 demonstrated a protective effect against ASA- and complement mediated injury to human sperm (D'Cruz, O. J., et al., Biol. Reprod. 54:1217-1228, 1996). These data provide several lines of evidence for the use of complement inhibitors in the treatment of urogenital disease and disorders.

An aspect of the invention thus provides a method for inhibiting lectin-dependent complement activation in a patient suffering from a urogenital disorder, by administering a composition comprising a therapeutically effective amount of a MAp19 inhibitory agent in a pharmaceutical carrier to a subject suffering from such a disorder. Urogenital disorders believed to be subject to therapeutic treatment with the methods and compositions of the present invention include, by way of nonlimiting example, painful bladder disease, sensory bladder disease, chronic abacterial cystitis and interstitial cystitis, male and female infertility, placental dysfunction and miscarriage and pre-eclampsia. The MAp19 inhibitory agent may be administered to the subject systemically, such as by intra-arterial, intravenous, intramuscular, inhalational, subcutaneous or other parenteral administration, or potentially by oral administration for non-peptidergic agents. Alternately, the MAp19 inhibitory composition may be delivered locally to the urogenital tract, such as by intravesical irrigation or instillation with a liquid solution or gel composition. Repeated administration may be carried out as determined by a physician to control or resolve the condition.

Diabetes and Diabetic Conditions

Diabetic retinal microangiopathy is characterized by increased permeability, leukostasis, microthrombosis, and apoptosis of capillary cells, all of which could be caused or promoted by activation of complement. Glomerular structures and endoneurial microvessels of patients with diabetes show signs of complement activation. Decreased availability or effectiveness of complement inhibitors in diabetes has been suggested by the findings that high glucose in vitro selectively decreases on the endothelial cell surface the expression of CD55 and CD59, the two inhibitors that are glycosylphosphatidylinositol (GPI)-anchored membrane proteins, and that CD59 undergoes nonenzymatic glycation that hinders its complement-inhibitory function.

Studies by Zhang et al. (Diabetes 51:3499-3504, 2002), investigated complement activation as a feature of human nonproliferative diabetic retinopathy and its association with changes in inhibitory molecules. It was found that deposition of C5b-9, the terminal product of complement activation, occurs in the wall of retinal vessels of human eye donors with type-2 diabetes, but not in the vessels of age-matched nondiabetic donors. C1q and C4, the complement components unique to the classical pathway, were not detected in the diabetic retinas, which indicates that C5b-9 was generated via the alternative pathway. The diabetic donors showed a prominent reduction in the retinal levels of CD55 and CD59, the two complement inhibitors linked to the plasma membrane by GPI anchors. Similar complement activation in retinal vessels and selective reduction in the levels of retinal CD55 and CD59 were observed in rats with a 10 week duration of streptozotocin-induced diabetes. Thus, diabetes appears to cause defective regulation of complement inhibitors and complement activation that precede most other manifestations of diabetic retinal microangiopathy.

Gerl et al. (Investigative Ophthalmology and Visual Science 43:1104-08, 2000) determined the presence of activated complement components in eyes affected by diabetic retinopathy. Immunohistochemical studies found extensive deposits of complement C5b-9 complexes that were detected in the choriocapillaris immediately underlying the Bruch membrane and densely surrounding the capillaries in all 50 diabetic retinopathy specimens. Staining for C3d positively correlated with C5b-9 staining, indicative of the fact that complement activation had occurred in situ. Furthermore, positive staining was found for vitronectin, which forms stable complexes with extracellular C5b-9. In contrast, there was no positive staining for C-reactive protein (CRP), mannan-binding lectin (MBL), C1q, or C4, indicating that complement activation did not occur through a C4-dependent pathway. Thus, the presence of C3d, C5b-9, and vitronectin indicates that complement activation occurs to completion, possibly through the alternative pathway in the choriocapillaris in eyes affected by diabetic retinopathy. Complement activation may be a causative factor in the pathologic sequelae that can contribute to ocular tissue disease and visual impairment. Therefore, the use of a complement inhibitor may be an effective therapy to reduce or block damage to microvessels that occurs in diabetes.

Insulin dependent diabetes mellitus (IDDM, also referred to as Type-I diabetes) is an autoimmune disease associated with the presence of different types of autoantibodies (Nicoloff et al., Clin. Dev. Immunol. 11:61-66, 2004). The presence of these antibodies and the corresponding antigens in the circulation leads to the formation of circulating immune complexes (CIC), which are known to persist in the blood for long periods of time. Deposition of CIC in the small blood vessels has the potential to lead to microangiopathy with debilitating clinical consequences. A correlation exists between CIC and the development of microvascular complications in diabetic children. These findings suggest that elevated levels of CIC IgG are associated with the development of early diabetic nephropathy and that an inhibitor of the complement pathway may be effective at blocking diabetic nephropathy (Kotnik et al., Croat. Med. J. 44:707-11, 2003). In addition, the formation of downstream complement proteins and the involvement of the alternative pathway is likely to be a contributory factor in overall islet cell function in IDDM, and the use of a complement inhibitor to reduce potential damage or limit cell death is expected (Caraher et al., J. Endocrinol. 162:143-53, 1999).

In another aspect of the invention, methods are provided for inhibiting lectin-dependent complement activation in a subject suffering from nonobese diabetes (IDDM) or from angiopathy, neuropathy or retinopathy complications of IDDM or adult onset (Type-2) diabetes, by administering a composition comprising a therapeutically effective amount of a MAp19 inhibitor in a pharmaceutical carrier. The MAp19 inhibitory agent may be administered to the subject systemically, such as by intra-arterial, intravenous, intramuscular, subcutaneous or other parenteral administration, or potentially by oral administration for non-peptidergic agents. Alternatively, administration may be by local delivery to the site of angiopathic, neuropathic or retinopathic symptoms. The MAp19 inhibitory agent may be administered periodically over an extended period of time for treatment or control of a chronic condition, or by a single or series of administrations for treatment of an acute condition.

Perichemotherapeutic Administration and Treatment of Malignancies

Activation of the complement system may also be implicated in the pathogenesis of malignancies. Recently, the neoantigens of the C5b-9 complement complex, IgG, C3, C4, S-protein/vitronectin, fibronectin, and macrophages were localized on 17 samples of breast cancer and on 6 samples of benign breast tumors using polyclonal or monoclonal antibodies and the streptavidin-biotin-peroxidase technique. All the tissue samples with carcinoma in each the TNM stages presented C5b-9 deposits on the membranes of tumor cells, thin granules on cell remnants, and diffuse deposits in the necrotic areas (Niculescu, F., et al., Am. J. Pathol. 140:1039-1043, 1992).

In addition, complement activation may be a consequence of chemotherapy or radiation therapy and thus inhibition of complement activation would be useful as an adjunct in the treatment of malignancies to reduce iatrogenic inflammation. When chemotherapy and radiation therapy preceded surgery, C5b-9 deposits were more intense and extended. The C5b-9 deposits were absent in all the samples with benign lesions. S-protein/vitronectin was present as fibrillar deposits in the connective tissue matrix and as diffuse deposits around the tumor cells, less intense and extended than fibronectin. IgG, C3, and C4 deposits were present only in carcinoma samples. The presence of C5b-9 deposits is indicative of complement activation and its subsequent pathogenetic effects in breast cancer (Niculescu, F., et al., Am. J. Pathol. 140:1039-1043, 1992).

Pulsed tunable dye laser (577 nm) (PTDL) therapy induces hemoglobin coagulation and tissue necrosis, which is mainly limited to blood vessels. In a PTDL-irradiated normal skin study, the main findings were as follows: 1) C3 fragments, C8, C9, and MAC were deposited in vessel walls; 2) these deposits were not due to denaturation of the proteins since they became apparent only 7 min after irradiation, contrary to immediate deposition of transferrin at the sites of erythrocyte coagulates; 3) the C3 deposits were shown to amplify complement activation by the alternative pathway, a reaction which was specific since tissue necrosis itself did not lead to such amplification; and 4) these reactions preceded the local accumulation of polymorphonuclear leucocytes. Tissue necrosis was more pronounced in the hemangiomas. The larger angiomatous vessels in the center of the necrosis did not fix complement significantly. By contrast, complement deposition in the vessels situated at the periphery was similar to that observed in normal skin with one exception: C8, C9, and MAC were detected in some blood vessels immediately after laser treatment, a finding consistent with assembly of the MAC occurring directly without the formation of a C5 convertase. These results indicate that complement is activated in PTDL-induced vascular necrosis, and might be responsible for the ensuing inflammatory response.

Photodynamic therapy (PDT) of tumors elicits a strong host immune response, and one of its manifestations is a pronounced neutrophilia. In addition to complement fragments (direct mediators) released as a consequence of PDT-induced complement activation, there are at least a dozen secondary mediators that all arise as a result of complement activity. The latter include cytokines IL-1beta, TNF-alpha, IL-6, IL-10, G-CSF and KC, thromboxane, prostaglandins, leukotrienes, histamine, and coagulation factors (Cecic, I., et al., Cancer Lett. 183:43-51, 2002).

Finally, the use of inhibitors of MAp19 for the inhibition of lectin-dependent complement activation may be envisioned in conjunction with the standard therapeutic regimens for the treatment of cancer. For example, treatment with rituximab, a chimeric anti-CD20 monoclonal antibody, can be associated with moderate to severe first-dose side-effects, notably in patients with high numbers of circulating tumor cells. Recent studies during the first infusion of rituximab measured complement activation products (C3b/c and C4b/c) and cytokines (tumour necrosis factor alpha (TNF-alpha), interleukin 6 (IL-6) and IL-8) in five relapsed low-grade non-Hodgkin's lymphoma (NHL) patients. Infusion of rituximab induced rapid complement activation, preceding the release of TNF-alpha, IL-6 and IL-8. Although the study group was small, the level of complement activation appeared to be correlated both with the number of circulating B cells prior to the infusion (r=0.85; P=0.07), and with the severity of the side-effects. The results indicated that complement plays a pivotal role in the pathogenesis of side-effects of rituximab treatment. As complement activation cannot be prevented by corticosteroids, it may be relevant to study the possible role of complement inhibitors during the first administration of rituximab (van der Kolk, L. E., et al., Br. J. Haematol. 115: 807-811, 2001).

In another aspect of the invention, methods are provided for inhibiting lectin-dependent complement activation in a subject being treated with chemotherapeutics and/or radiation therapy, including, without limitation, for the treatment of cancerous conditions. This method includes administering a composition comprising a therapeutically effective amount of a MAp19 inhibitor in a pharmaceutical carrier to a patient perichemotherapeutically, i.e., before and/or during and/or after the administration of chemotherapeutic(s) and/or radiation therapy. For example, administration of a MAp19 inhibitor composition of the present invention may be commenced before or concurrently with the administration of chemo- or radiation therapy, and continued throughout the course of therapy, to reduce the detrimental effects of the chemo- and/or radiation therapy in the non-targeted, healthy tissues. In addition, the MAp19 inhibitor composition can be administered following chemo- and/or radiation therapy. It is understood that chemo- and radiation therapy regimens often entail repeated treatments and, therefore, it is possible that administration of a MAp19 inhibitor composition would also be repetitive and relatively coincident with the chemotherapeutic and radiation treatments. It is also believed that MAp19 inhibitory agents may be used as chemotherapeutic agents, alone or in combination with other chemotherapeutic agents and/or radiation therapy, to treat patients suffering from malignancies. Administration may suitably be via oral (for non-peptidergic), intravenous, intramuscular or other parenteral route.

Endocrine Disorders

The complement system has also been recently associated with a few endocrine conditions or disorders including Hashimoto's thyroiditis (Blanchin, S., et al., Exp. Eye Res. 73(6):887-96, 2001), stress, anxiety and other potential hormonal disorders involving regulated release of prolactin, growth or insulin-like growth factor, and adrenocorticotropin from the pituitary (Francis, K, et al., FASEB J. 17:2266-2268, 2003; Hansen, T. K., Endocrinology 144(12):5422-9, 2003).

Two-way communication exists between the endocrine and immune systems using molecules such as hormones and cytokines. Recently, a new pathway has been elucidated by which C3a, a complement-derived cytokine, stimulates anterior pituitary hormone release and activates the hypothalamic-pituitary-adrenal axis, a reflex central to the stress response and to the control of inflammation. C3a receptors are expressed in pituitary-hormone-secreting and non-hormone-secreting (folliculostellate) cells. C3a and C3adesArg (a non-inflammatory metabolite) stimulate pituitary cell cultures to release prolactin, growth hormone, and adrenocorticotropin. Serum levels of these hormones, together with adrenal corticosterone, increase dose dependently with recombinant C3a and C3adesArg administration in vivo. The implication is that complement pathway modulates tissue-specific and systemic inflammatory responses through communication with the endocrine pituitary gland (Francis, K., et al., FASEB J. 17:2266-2268, 2003).

An increasing number of studies in animals and humans indicate that growth hormone (GH) and insulin-like growth factor-I (IGF-I) modulate immune function. GH therapy increased the mortality in critically ill patients. The excessive mortality was almost entirely due to septic shock or multi-organ failure, which could suggest that a GH-induced modulation of immune and complement function was involved. Mannan-binding lectin (MBL) is a plasma protein that plays an important role in innate immunity through activation of the complement cascade and inflammation following binding to carbohydrate structures. Evidence supports a significant influence from growth hormone on MBL levels and, therefore, potentially on lectin-dependent complement activation (Hansen, T. K., Endocrinology 144(12):5422-9, 2003).

Thyroperoxidase (TPO) is one of the main autoantigens involved in autoimmune thyroid diseases. TPO consists of a large N-terminal myeloperoxidase-like module followed by a complement control protein (CCP)-like module and an epidermal growth factor-like module. The CCP module is a constituent of the molecules involved in the activation of C4 complement component, and studies were conducted to investigate whether C4 may bind to TPO and activate the complement pathway in autoimmune conditions. TPO via its CCP module directly activates complement without any mediation by Ig. Moreover, in patients with Hashimoto's thyroiditis, thyrocytes overexpress C4 and all the downstream components of the complement pathway. These results indicate that TPO, along with other mechanisms related to activation of the complement pathway, may contribute to the massive cell destruction observed in Hashimoto's thyroiditis (Blanchin, S., et al., 2001).

An aspect of the invention thus provides a method for inhibiting lectin-dependent complement activation to treat an endocrine disorder, by administering a composition comprising a therapeutically effective amount of a MAp19 inhibitory agent in a pharmaceutical carrier to a subject suffering from an endocrine disorder. Conditions subject to treatment in accordance with the present invention include, by way of nonlimiting example, Hashimoto's thyroiditis, stress, anxiety and other potential hormonal disorders involving regulated release of prolactin, growth or insulin-like growth factor, and adrenocorticotropin from the pituitary. The MAp19 inhibitory agent may be administered to the subject systemically, such as by intra-arterial, intravenous, intramuscular, inhalational, nasal, subcutaneous or other parenteral administration, or potentially by oral administration for non-peptidergic agents. The MAp19 inhibitory agent composition may be combined with one or more additional therapeutic agents. Administration may be repeated as determined by a physician until the condition has been resolved.

Ophthalmologic Conditions

Age-related macular degeneration (AMD) is a blinding disease that afflicts millions of adults, yet the sequelae of biochemical, cellular, and/or molecular events leading to the development of AMD are poorly understood. AMD results in the progressive destruction of the macula which has been correlated with the formation of extracellular deposits called drusen located in and around the macula, behind the retina and between the retina pigment epithelium (RPE) and the choroid. Recent studies have revealed that proteins associated with inflammation and immune-mediated processes are prevalent among drusen-associated constituents. Transcripts that encode a number of these molecules have been detected in retinal, RPE, and choroidal cells. These data also demonstrate that dendritic cells, which are potent antigen-presenting cells, are intimately associated with drusen development, and that complement activation is a key pathway that is active both within drusen and along the RPE-choroid interface (Hageman, G. S., et al., Prog. Retin. Eye Res. 20:705-732, 2001).

Several independent studies have shown a strong association between AMD and a genetic polymorphism in the gene for complement factor H (CFH), in which the likelihood of AMD is increased by a factor of 7.4 in individuals homozygous for the risk allele (Klein, R. J., et al., Science 308:362-364, 2005; Haines et al., Science 308:362-364. 2005; Edwards et al., Science 308:263-264, 2005). The CFH gene has been mapped to chromosome 1q31, a region that had been implicated in AMD by six independent linkage scans (see, e.g., D. W. Schultz, et al., Hum. Mol. Genet. 12:3315, 2003). CFH is known to be a key regulator of the complement system. It has been shown that CFH on cells and in circulation regulates complement activity by inhibiting the activation of C3 to C3a and C3b, and by inactivating existing C3b. Deposition of C5b-9 has been observed in Brusch's membrane, the intercapillary pillars and within drusen in patients with AMD (Klein et al.). Immunofluorescence experiments suggest that in AMD, the polymorphism of CFH may give rise to complement deposition in chorodial capillaries and chorodial vessels (Klein et al). The membrane-associated complement inhibitor, complement receptor 1, is also localized in drusen, but it is not detected in RPE cells immunohistochemically. In contrast, a second membrane-associated complement inhibitor, membrane cofactor protein, is present in drusen-associated RPE cells, as well as in small, spherical substructural elements within drusen. These previously unidentified elements also show strong immunoreactivity for proteolytic fragments of complement component C3 that are characteristically deposited at sites of complement activation. It is proposed that these structures represent residual debris from degenerating RPE cells that are the targets of complement attack (Johnson, L. V., et al., Exp. Eye Res. 73:887-896, 2001).

An aspect of the invention thus provides a method for inhibiting lectin-dependent complement activation to treat age-related macular degeneration or other complement mediated ophthalmologic condition by administering a composition comprising a therapeutically effective amount of a MAp19 inhibitory agent in a pharmaceutical carrier to a subject suffering from such a condition or other complement-mediated ophthalmologic condition. The MAp19 inhibitory composition may be administered locally to the eye, such as by irrigation or application of the composition in the form of a gel, salve or drops. Alternately, the MAp19 inhibitory agent may be administered to the subject systemically, such as by intra-arterial, intravenous, intramuscular, inhalational, nasal, subcutaneous or other parenteral administration, or potentially by oral administration for non-peptidergic agents. The MAp19 inhibitory agent composition may be combined with one or more additional therapeutic agents, such as are disclosed in US Patent Application Publication Number 2004-0072809-A1. Administration may be repeated as determined by a physician until the condition has been resolved or is controlled.

IV. MAp19 Inhibitory Agents

In one aspect, the present invention provides methods of inhibiting the effects of lectin-dependent complement activation. MAp19 inhibitory agents are administered in an amount effective to inhibit lectin-dependent complement activation in a living subject. In the practice of this aspect of the invention, representative MAp19 inhibitory agents include: molecules that inhibit the biological activity of MAp19 (such as small molecule inhibitors, anti-MAp19 antibodies or blocking peptides which interact with MAp19 or interfere with a protein-protein interaction), and molecules that decrease the expression of MAp19 and/or MASP-2 (such as MAp19 antisense nucleic acid molecules, MAp19 specific RNAi molecules and MAp19 ribozymes), thereby preventing MAp19 and/or MASP-2 from activating the lectin-dependent complement pathway. The MAp19 inhibitory agents can be used alone as a primary therapy or in combination with other therapeutics as an adjuvant therapy to enhance the therapeutic benefits of other medical treatments.

The inhibition of lectin complement activation is characterized by at least one of the following changes in a component of the complement system that occurs as a result of administration of a MAp19 inhibitory agent in accordance with the methods of the invention: the inhibition of the generation or production of lectin-dependent complement activation system products C4b, C3a, C5a and/or C5b-9 (MAC) (measured, for example, as described in Example 2), the reduction of alternative complement activation assessed in a hemolytic assay using unsensitized rabbit or guinea pig red blood cells, the reduction of C4 cleavage and C4b deposition (measured, for example as described in Example 2), or the reduction of C3 cleavage and C3b deposition (measured, for example, as described in Example 4).

In one embodiment, a MAp19 inhibitory agent useful in the methods of the invention is a specific MAp19 inhibitory agent that specifically binds to a polypeptide comprising SEQ ID NO:3 with an affinity of at least 10 times greater than to other antigens in the complement system. In another embodiment, a MAp19 inhibitory agent specifically binds to a polypeptide comprising SEQ ID NO:3 with a binding affinity of at least 100 times greater than to other antigens in the complement system. The binding affinity of the MAp19 inhibitory agent can be determined using the binding assay described in Example 9.

According to the present invention, MAp19 inhibitory agents are utilized that are effective in inhibiting the lectin-dependent complement activation system. MAp19 inhibitory agents useful in the practice of this aspect of the invention include, for example, anti-MAp19 antibodies and fragments thereof, MAp19 inhibitory peptides, small molecules, MAp19 soluble receptors and expression inhibitors. MAp19 inhibitory agents may inhibit the lectin-dependent complement activation system by blocking the biological function of MAp19. For example, an inhibitory agent may effectively block MAp19 protein-to-protein interactions, interfere with MAp19 dimerization, or assembly, block Ca²⁺ binding, or may reduce MAp19 and/or MASP-2 protein expression. In some embodiments, the MAp19 inhibitory agents selectively inhibit lectin-dependent complement activation, leaving the C1q-dependent complement activation system functionally intact. In one embodiment, the MAp19 inhibitory agent specifically binds to or interacts with MAp19 or a portion thereof and does not bind to MASP-2.

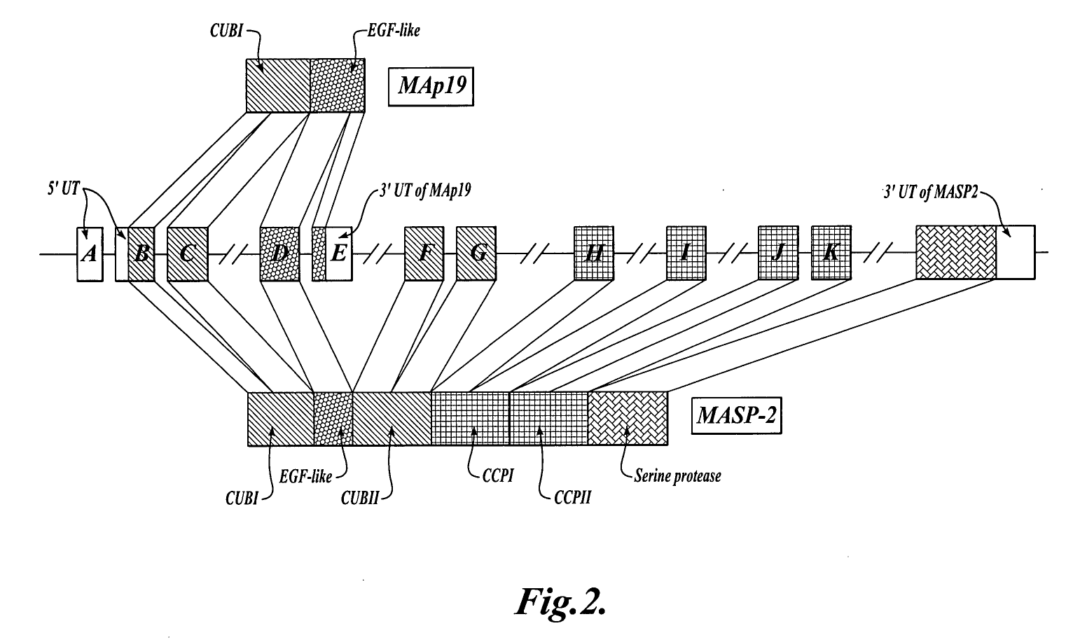

The MAp19 polypeptide exhibits a molecular structure similar to MASP-2, MASP-1, MASP-3, and C1r and C1s, the proteases of the C1 complement system. The MAp19 cDNA arises from an alternative splicing of the MASP 2 gene. The cDNA molecule set forth in SEQ ID NO:1 encodes a representative example of human MAp19 (consisting of the amino acid sequence set forth in SEQ ID NO:2) and provides the MAp19 polypeptide with a leader sequence (aa 1-15) that is cleaved after secretion, resulting in the mature form of MAp19 (SEQ ID NO:3). As shown in FIG. 2, the MAp19 is encoded by exons B, C, D and E of the MASP 2 gene, whereas MASP-2 cDNA is encoded by exons B, C, D, F, G, H, I, J, K and L. Therefore, the C-terminus of MAp19 comprising the amino acid sequence “EQSL” encoded by exon E is unique to MAp19 and is not contained in MASP-2.

Those skilled in the art will recognize that the sequences disclosed in SEQ ID NO:1 represent single alleles of human MAp19 and that allelic variation is expected to occur. Allelic variants of the nucleotide sequences shown in SEQ ID NO:1, including those containing silent mutations and those in which mutations result in amino acid sequence changes, are within the scope of the present invention. Allelic variants of the MAp19 sequence can be cloned by probing cDNA or genomic libraries from different individuals according to standard procedures.

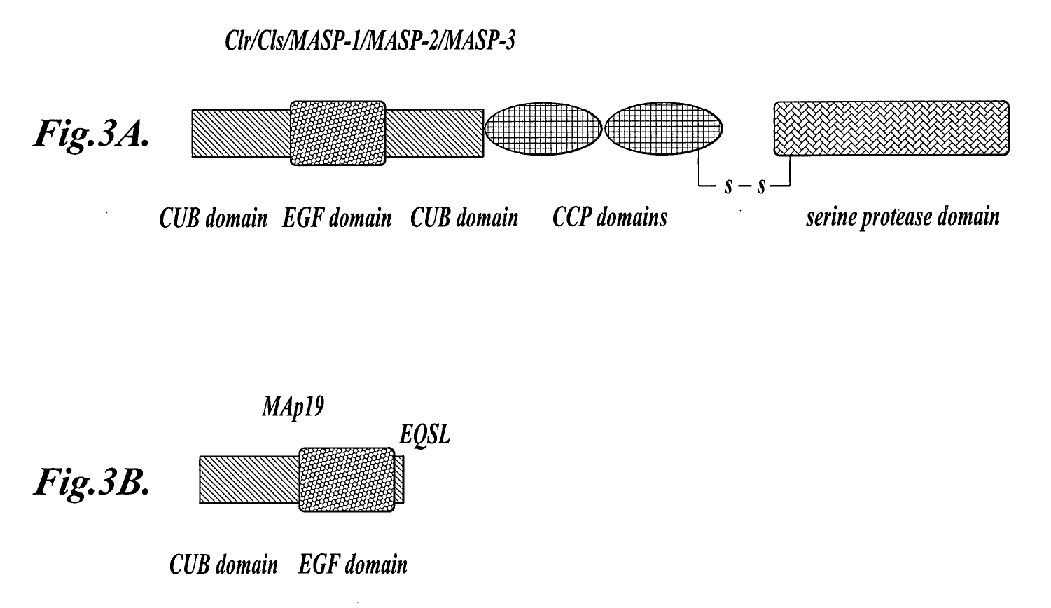

The domains of the MAp19 protein (SEQ ID NO:3) are shown in FIG. 3B and include an N-terminal C1r/C1s/sea urchin Vegf/bone morphogenic protein (CUBI) domain (aa 1-121 of SEQ ID NO:3), and an epidermal growth factor-like domain (aa 122-166) prolonged by four unique residues at its C-terminal end (Glu, Gln, Ser and Leu) that are not present in MASP-2 (shown in FIG. 3A).

The structure of human MAp19 has been solved by X-ray crystallography (Gregory, L. A., et al., J. Biol. Chem., 2004, supra). MAp19 forms a head to tail homodimer held together by interactions between the CUB1 domain of one monomer and the EGF domain of the counterpart. A Ca⁺⁺ ion bound to each EGF domain stabilizes the dimer interfaces. A second Ca⁺⁺ ion is bound to the distal end of each CUB1 domain, through six ligands contributed by Glu 52, Asp60, Asp105, Ser107 Asn108 and a water molecule.

Several proteins have been shown to bind to, or interact with, MAp19 through protein-to-protein interactions. For example, MAp19 is known to bind to and form Ca²⁺ dependent complexes with the lectin proteins MBL, H-ficolin and L-ficolin (Stover, C. M., J. Immunol. 162:3481-90, 1999). It has also been shown that MAp19 binds to MBL with lower affinity than the full size MASP-2 because it lacks the second CUB domain that is directly involved in the interaction (Thielens, N. M., et al., J. Immunol. 166:5068-77, 2001; Chen, C. B. and Wallis, R., J. Biol. Chem. 276:25894-902, 2001). In a recent study, site-directed mutagenesis was used to identify the residues on MAp19 involved in the interaction with MBL and L-ficolin (Gregory et al., 2004, supra). The study showed that mutations at Tyr59, Asp60, Glu83, Asp105, Tyr106 and Glu109 of SEQ ID NO:3 either strongly decreased or abolished interaction with both MBL and L-ficolin. These mutations map a common binding site for these proteins located at the distal end of the MAp19 CUB1 module which are stabilized by the Ca⁺⁺ ion. The X-ray crystallography model for this proposed MBL binding site shows that side chains of Tyr59, Glu83, Tyr106 and Glu109 all protrude from the CUBI module and are available for binding in protein-protein interactions (Gregory et al., 2004, supra). Consistent with these results, a naturally occurring mutation Asp105Gly has been described recently in human MASP-2 (which is a region also present in MAp19), and shown to result in a complete loss of interaction of both MASP-2 and MAp19 with MBL (Stengaard-Pedersen et al., 2003). Therefore, MAp19 inhibitory agents can be identified that are useful in the method of the invention that bind to or interfere with MAp19 target regions known to be important for MAp19-dependent complement activation.

In one embodiment of this aspect of the invention the MAp19 inhibitory agents specifically bind to or interact with MAp19 or a portion thereof and do not bind to MASP-2. MAp19 contains a unique 4 amino acid extension at its C-terminus. Therefore, the sequence “SEQSL” (SEQ ID NO:12) comprises a unique linear epitope for MAp19 that is not present in MASP-2. In addition, due to the presence of the unique C-terminus and smaller size without the additional domains of MASP-2, MAp19 likely folds into a different three dimensional conformation than MASP-2, thereby producing MAp19 conformational epitopes that differ from MASP-2. There is evidence in the literature that MAp19 may complex with different proteins than does MASP-2, further supporting the ability to derive MAp19 specific inhibitors that do not bind to MASP-2. Moreover, in the X-ray crystallography study of MAp19 published by Gregory et al., significant differences were observed between the structure of human MAp19 and rat MASP-2 with respect to the relative positioning of their modules, both at the intra and inter-monomer levels. For example, the MAp19 homodimer was shown to have a unique topology in which its two N-terminal segments, despite their identical amino acid sequences, exhibit different conformations and interact with each other in an asymmetrical manner (Gregory et al., 2004, supra). A similar ordered structure was not observed in the rat MASP-2 crystal structure (Feinberg et al., EMBO J. 22:2348-2359). Therefore, agents that specifically bind to MAp19 and which do not bind to MASP-2 may be obtained, for example using the methods described herein.

### Anti-MAp19 Antibodies

In some embodiments, the MAp19 inhibitory agent comprises an anti-MAp19 antibody that inhibits the lectin-dependent complement activation system. The anti-MAp19 antibodies useful in this aspect of the invention include polyclonal, monoclonal, or recombinant antibodies derived from any antibody producing mammal and may be multispecific, chimeric, humanized, antiidiotype, and antibody fragments. Antibody fragments include Fab, Fab′, F(ab)₂, F(ab′)₂, Fv fragments, scFv fragments and single-chain antibodies as further described herein.

Several anti-MAp19 antibodies have been described in the literature, some of which are listed below in TABLE 1. Although the anti-MAp19 antibodies described in the literature were raised using MAp19 antigens, these described antibodies also cross-react with MASP-2. These previously described anti-MAp19 antibodies can be screened for the ability to inhibit the lectin-dependent complement activation system using the assays described herein. Once an anti-MAp19 antibody is identified that functions as a MAp19 inhibitory agent, it can be used to produce antiidiotype antibodies and used to identify other MAp19 binding molecules as further described below.

TABLE 1
MAP19 SPECIFIC ANTIBODIES FROM THE LITERATURE

| Antigen | Antibody Type | Reference |
|---|---|---|
| MAp19 N-terminal | Rabbit polyclonal | Kang et al., FEBS Letters |
| Peptide (cross reacts | | 462:89-93, 1999. |
| with MASP-2) | | |
| Rhuman MAp 19 | Rat MoAb (subclass | Moller-Kristensen, M., et al., |
| (MoAb 6G12) | IgG1) | J. Immunol. Methods 282: |
| (cross-reacts with | | 159-167, 2003. |
| MASP-2) | | |

Anti-MAp19 Antibodies with Reduced Effector Function

In some embodiments of this aspect of the invention, the anti-MAp19 antibodies have reduced effector function in order to reduce inflammation that may arise from the activation of the classical complement pathway. The ability of IgG molecules to trigger the classical complement pathway has been shown to reside within the Fc portion of the molecule (Duncan, A. R., et al., Nature 332:738-740, 1988). IgG molecules in which the Fc portion of the molecule has been removed by enzymatic cleavage are devoid of this effector function (see Harlow and Lane, Antibodies: A Laboratory Manual, Cold Spring Harbor Laboratory, New York, 1988). Accordingly, antibodies with reduced effector function can be generated as the result of lacking the Fc portion of the molecule, by having a genetically engineered Fc sequence that minimizes effector function, or being of either the human IgG₂ or IgG₄ isotype.

Antibodies with reduced effector function can be produced by standard molecular biological manipulation of the Fc portion of the IgG heavy chains as described in Example 13 herein and also described in Jolliffe et al., Int'l Rev. Immunol. 10:241-250, 1993, and Rodrigues et al., J. Immunol. 151:6954-6961, 1998. Antibodies with reduced effector function also include human IgG2 and IgG4 isotypes that have a reduced ability to activate complement and/or interact with Fc receptors (Ravetch, J. V., et al., Annu. Rev. Immunol. 9:457-492, 1991; Isaacs, J. D., et al., J. Immunol. 148:3062-3071, 1992; van de Winkel, J. G., et al., Immunol. Today 14:215-221, 1993). Humanized or fully human antibodies specific to human MAp19 comprised of IgG2 or IgG4 isotypes can be produced by one of several methods known to one of ordinary skill in the art, as described in Vaughan, T. J., et al., Nature Biotechnical 16:535-539, 1998.

### Production of Anti-MAp19 Antibodies

Anti-MAp19 antibodies can be produced using MAp19 polypeptides (e.g., full length MAp19) or using antigenic MAp19-epitope bearing peptides (e.g., a portion of the MAp19 polypeptide). Immunogenic peptides may be as small as five amino acid residues. For example, the MAp19 polypeptide including the entire amino acid sequence of SEQ ID NO:3 may be used to induce anti-MAp19 antibodies useful in the method of the invention. Particular MAp19 domains known to be involved in protein-protein interactions, such as the CUBI, and CUBIEGF domains may be expressed as recombinant polypeptides and used as antigens. In addition, peptides comprising a portion of at least 6 amino acids of the MAp19 polypeptide (SEQ ID NO:3) are also useful to induce MAp19 antibodies. Additional examples of MAp19 derived antigens useful to induce MAp19 antibodies are provided below in TABLE 2. The MAp19 peptides and polypeptides used to raise antibodies may be isolated as natural polypeptides, or recombinant or synthetic peptides, as further described herein. In some embodiments of this aspect of the invention, anti-MAp19 antibodies are obtained using a transgenic mouse strain as described in Examples 10 and 11.

In one embodiment, the anti-MAp19 antibodies useful in the method of the invention specifically bind to MAp19 and do not bind to MASP-2 and may be obtained using the murine strains deficient in MAp19 and/or MASP-2 using the methods described herein.

Antigens useful for producing anti-MAp19 antibodies also include fusion polypeptides, such as fusions of MAp19 or a portion thereof, with an immunoglobulin polypeptide or with maltose-binding protein. The polypeptide immunogen may be a full-length molecule or a portion thereof. If the polypeptide portion is hapten-like, such portion may be advantageously joined or linked to a macromolecular carrier (such as keyhole limpet hemocyanin (KLH), bovine serum albumin (BSA) or tetanus toxoid) for immunization.

TABLE 2
MAP19 DERIVED ANTIGENS

| | |
|---|---|
| SEQ ID NO: | Amino Acid Sequence |

| | |
|---|---|
| SEQ ID NO:3 | Human MAp19 protein |
| SEQ ID NO:45 | Murine MAp19 protein |
| SEQ ID NO:8 | CUBI domain of MAp19 |
| (aa 1-121 of SEQ ID NO:3) | |
| SEQ ID NO:9 | CUBI peptide |
| TPLGPKWPEPVFGRL | (N-terminus) |
| SEQ ID NO:10 | CUBI peptide |
| TAPPGYRLRLYFTHFDLELSHLCEYD | (dimerization region) |
| FVKLSSGAKVLATLCGQ | |
| SEQ ID NO:11 | MBL binding region |
| DTERAPGKDTFYSLGSSLDITFRSDY | in CUBI domain |
| SNEKPFTGF | |
| SEQ ID NO:12 | MAp19 unique C-terminus |
| SEQSL | |
| SEQ ID NO:13 | Unique MAp19 C-terminal |
| APTCGHHCHNHLGGFYCSCRAGYVLH | peptide and inter- |
| RNKRTCSEQSL | monomer interface |

### Polyclonal Antibodies

Polyclonal antibodies against MAp19 can be prepared by immunizing an animal with MAp19 polypeptide, or an immunogenic portion, thereof using methods well-known to those of ordinary skill in the art. See, for example, Green, et al., “Production of Polyclonal Antisera,” in Immunochemical Protocols, Manson (ed.), page 105, and as further described in Example 8. The immunogenicity of a MAp19 polypeptide can be increased through the use of an adjuvant, including mineral gels, such as aluminum hydroxide or Freund's adjuvant (complete or incomplete), surface active substances such as lysolecithin, pluronic polyols, polyanions, oil emulsions, keyhole limpet hemocyanin and dinitrophenol. Polyclonal antibodies are typically raised in animals such as horses, cows, dogs, chicken, rats, mice, rabbits, guinea pigs, goats, or sheep. Alternatively, an anti-MAp19 antibody useful in the present invention may also be derived from a subhuman primate. General techniques for raising diagnostically and therapeutically useful antibodies in baboons may be found, for example, in Goldenberg et al., International Patent Publication No. WO 91/11465, and in Losman, M. J., et al., Int. J. Cancer 46:310, 1990. Sera containing immunologically active antibodies are then produced from the blood of such immunized animals using standard procedures well known in the art.

### Monoclonal Antibodies

In some embodiments, the MAp19 inhibitory agent is an anti-MAp19 monoclonal antibody. Anti-MAp19 monoclonal antibodies are highly specific, being directed against a single MAp19 epitope. In some embodiments, the anti-MAp19 antibodies do not bind to MASP-2. As used herein, the modifier “monoclonal” indicates the character of the antibody as being obtained from a substantially homogenous population of antibodies, and is not to be construed as requiring production of the antibody by any particular method. Monoclonal antibodies can be obtained using any technique that provides for the production of antibody molecules by continuous cell lines in culture, such as the hybridoma method described by Kohler, G., et al., Nature 256:495, 1975, or they may be made by recombinant DNA methods (see, e.g., U.S. Pat. No. 4,816,567 to Cabilly). Monoclonal antibodies may also be isolated from phage antibody libraries using the techniques described in Clackson, T., et al., Nature 352:624-628, 1991, and Marks, J. D., et al., J. Mol. Biol. 222:581-597, 1991. Such antibodies can be of any immunoglobulin class including IgG, IgM, IgE, IgA, IgD and any subclass thereof.

For example, monoclonal antibodies can be obtained by injecting a suitable mammal (e.g., a BALB/c mouse) with a composition comprising a MAp19 polypeptide portion thereof. After a predetermined period of time, splenocytes are removed from the mouse and suspended in a cell culture medium. The splenocytes are then fused with an immortal cell line to form a hybridoma. The formed hybridomas are grown in cell culture and screened for their ability to produce a monoclonal antibody against MAp19. An example further describing the production of anti-MAp19 monoclonal antibodies is provided in Example 9. See also, Current Protocols in Immunology, Vol. 1, pages 2.5.1-2.6.7, John Wiley & Sons, 1991.

Human monoclonal antibodies may be obtained through the use of transgenic mice that have been engineered to produce specific human antibodies in response to antigenic challenge. In this technique, elements of the human immunoglobulin heavy and light chain locus are introduced into strains of mice derived from embryonic stem cell lines that contain targeted disruptions of the endogenous immunoglobulin heavy chain and light chain loci. The transgenic mice can synthesize human antibodies specific for human antigens, such as the MAp19 antigens described herein, and the mice can be used to produce human MAp19 antibody-secreting hybridomas by fusing B-cells from such animals to suitable myeloma cell lines using conventional Kohler-Milstein technology as further described in Example 9. Transgenic mice with a human immunoglobulin genome are commercially available (e.g., from Abgenix, Inc., Fremont, Calif., and Medarex, Inc., Annandale, N.J.). Methods for obtaining human antibodies from transgenic mice are described, for example, by Green, L. L., et al., Nature Genet. 7:13, 1994; Lonberg, L., et al., Nature 368:856, 1994; and Taylor, L. D., et al., Int. Immun. 6:579, 1994.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Meth. Mol. Biol., 10:79-104, 1992).

Once produced, polyclonal, monoclonal or phage-derived antibodies are first tested for specific MAp19 binding. A variety of assays known to those skilled in the art may be utilized to detect antibodies which specifically bind to MAp19. Exemplary assays include Western blot or immunoprecipitation analysis by standard methods (e.g., as described in Ausubel et al.), immunoelectrophoresis, enzyme-linked immuno-sorbent assays, dot blots, inhibition or competition assays and sandwich assays (as described in Harlow and Land, Antibodies: A Laboratory Manual, Cold Spring Harbor Laboratory Press, 1988). Once antibodies are identified that specifically bind to MAp19, the anti-MAp19 antibodies are tested for the ability to function as a MAp19 inhibitory agent in one of several assays such as, for example, a lectin-specific C4 cleavage assay (described in Example 2), a C3b deposition assay (described in Example 4) or a C4b deposition assay (described in Example 2). The anti-MAp19 antibodies can also be tested for cross-reactivity with MASP-2 using a binding assay as described in Example 9.

The affinity of anti-MAp19 monoclonal antibodies can be readily determined by one of ordinary skill in the art (see, e.g., Scatchard, A., Ann. NY Acad. Sci. 51:660-672, 1949). In one embodiment, the anti-MAp19 monoclonal antibodies useful for the methods of the invention bind to MASP-2 with a binding affinity of <100 nM, preferably <10 nM, and most preferably <2 nM.

### Chimeric/Humanized Antibodies

Monoclonal antibodies useful in the method of the invention include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies (U.S. Pat. No. 4,816,567 to Cabilly, and Morrison, S. L., et al., Proc. Nat'l Acad. Sci. USA 81:6851-6855, 1984).

One form of a chimeric antibody useful in the invention is a humanized monoclonal anti-MAp19 antibody. Humanized forms of non-human (e.g., murine) antibodies are chimeric antibodies, which contain minimal sequences derived from non-human immunoglobulin. Humanized monoclonal antibodies are produced by transferring the non-human (e.g., mouse) complementarity determining regions (CDR), from the heavy and light variable chains of the mouse immunoglobulin into a human variable domain. Typically, residues of human antibodies are then substituted in the framework regions of the non-human counterparts. Furthermore, humanized antibodies may comprise residues that are not found in the recipient antibody or in the donor antibody. These modifications are made to further refine antibody performance.

In general, the humanized antibody will comprise substantially all of at least one, and typically two variable domains, in which all or substantially all of the hypervariable loops correspond to those of a non-human immunoglobulin and all or substantially all of the Fv framework regions are those of a human immunoglobulin sequence. The humanized antibody optionally also will comprise at least a portion of an immunoglobulin constant region (Fc), typically that of a human immunoglobulin. For further details, see Jones, P. T., et al., Nature 321:522-525, 1986; Reichmans, L., et al., Nature 332:323-329, 1988; and Presta, Curr. Op. Struct. Biol. 2:593-596, 1992.

The humanized antibodies useful in the invention include human monoclonal antibodies including at least a MAp19 binding CDR3 region. In addition, the Fc portions may be replaced so as to produce IgA or IgM as well as human IgG antibodies. Such humanized antibodies will have particular clinical utility because they will specifically recognize human MAp19 but will not evoke an immune response in humans against the antibody itself. Consequently, they are better suited for in vivo administration in humans, especially when repeated or long-term administration is necessary.

An example of the generation of a humanized anti-MAp19 antibody from a murine anti-MAp19 monoclonal antibody is provided herein in Example 13. Techniques for producing humanized monoclonal antibodies are also described, for example, by Jones, P. T., et al., Nature 321:522, 1986; Carter, P., et al., Proc. Nat'l. Acad. Sci. USA 89:4285, 1992; Sandhu, J. S., Crit. Rev. Biotech. 12:437, 1992; Singer, I. I., et al., J. Immun. 150:2844, 1993; Sudhir (ed.), Antibody Engineering Protocols, Humana Press, Inc., 1995; Kelley, “Engineering Therapeutic Antibodies,” in Protein Engineering: Principles and Practice, Cleland et al. (eds.), John Wiley & Sons, Inc., pages 399-434, 1996; and by U.S. Pat. No. 5,693,762 to Queen, 1997. In addition, there are commercial entities that will synthesize humanized antibodies from specific murine antibody regions, such as Protein Design Labs (Mountain View, Calif.).

### Recombinant Antibodies

Anti-MAp19 antibodies can also be made using recombinant methods. For example, human antibodies can be made using human immunoglobulin expression libraries (available for example, from Stratagene, Corp., La Jolla, Calif.) to produce fragments of human antibodies (V_{H}, V_{L}, Fv, Fd, Fab or F(ab′)₂). These fragments are then used to construct whole human antibodies using techniques similar to those for producing chimeric antibodies.

### Anti-Idiotype Antibodies

Once anti-MAp19 antibodies are identified with the desired inhibitory activity, these antibodies can be used to generate anti-idiotype antibodies that resemble a portion of MAp19 using techniques that are well known in the art. See, e.g., Greenspan et al., FASEB J. 7:437, 1993. For example, antibodies that bind to MAp19 and competitively inhibit a MAp19 protein interaction required for complement activation can be used to generate anti-idiotypes that resemble the MBL binding site on MAp19 protein and therefore bind and neutralize a binding ligand of MAp19 such as, for example, MBL.

### Immunoglobulin Fragments

The MAp19 inhibitory agents useful in the method of the invention encompass not only intact immunoglobulin molecules but also the well known fragments including Fab, Fab′, F(ab)₂, F(ab′)₂ and Fv fragments, scFv fragments, diabodies, linear antibodies, single-chain antibody molecules and multispecific antibodies formed from antibody fragments. It is well known in the art that only a small portion of an antibody molecule, the paratope, is involved in the binding of the antibody to its epitope (see, e.g., Clark, W. R., The Experimental Foundations of Modern Immunology, Wiley & Sons, Inc., NY, 1986). The pFc′ and Fc regions of the antibody are effectors of the classical complement pathway, but are not involved in antigen binding. An antibody from which the pFc′ region has been enzymatically cleaved, or which has been produced without the pFc′ region, is designated an F(ab′)₂ fragment and retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, is designated a Fab fragment, and retains one of the antigen binding sites of an intact antibody molecule.

Antibody fragments can be obtained by proteolytic hydrolysis, such as by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by U.S. Pat. No. 4,331,647 to Goldenberg; Nisonoff, A., et al., Arch. Biochem. Biophys. 89:230, 1960; Porter, R. R., Biochem. J. 73:119, 1959; Edelman et al., in Methods in Enzymology, 1:422, 1967; and by Coligan at pages 2.8.1-2.8.10 and 2.10.-2.10.4.

In some embodiments, the use of antibody fragments lacking the Fc region are preferred to avoid activation of the classical complement pathway which is initiated upon binding Fc to the Fcγ receptor. There are several methods by which one can produce a MoAb that avoids Fcγ receptor interactions. For example, the Fc region of a monoclonal antibody can be removed chemically using partial digestion by proteolytic enzymes (such as ficin digestion), thereby generating, for example, antigen-binding antibody fragments such as Fab or F(ab)₂ fragments (Mariani, M., et al., Mol. Immunol. 28:69-71, 1991). Alternatively, the human γ4 IgG isotype, which does not bind Fcγ receptors, can be used during construction of a humanized antibody as described herein. Antibodies, single chain antibodies and antigen-binding domains that lack the Fc domain can also be engineered using recombinant techniques described herein.

### Single-Chain Antibody Fragments

Alternatively, one can create single peptide chain binding molecules specific for MAp19 in which the heavy and light chain Fv regions are connected. The Fv fragments may be connected by a peptide linker to form a single-chain antigen binding protein (scFv). These single-chain antigen binding proteins are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described for example, by Whitlow et al., “Methods: A Companion to Methods in Enzymology” 2:97, 1991; Bird et al., Science 242:423, 1988; Ladner et al., U.S. Pat. No. 4,946,778; and Pack, P., et al., Bio/Technology 11: 1271, 1993.

As an illustrative example, a MAp19 specific scFv can be obtained by exposing lymphocytes to MAp19 polypeptide in vitro and selecting antibody display libraries in phage or similar vectors (for example, through the use of immobilized or labeled MAp19 protein or peptide). Genes encoding polypeptides having potential MAp19 polypeptide binding domains can be obtained by screening random peptide libraries displayed on phage or on bacteria such as E. coli. These random peptide display libraries can be used to screen for peptides which interact with MAp19. Techniques for creating and screening such random peptide display libraries are well known in the art (U.S. Pat. No. 5,223,409 to Lardner; U.S. Pat. No. 4,946,778 to Lardner; U.S. Pat. No. 5,403,484 to Lardner; U.S. Pat. No. 5,571,698 to Lardner and Kay et al., Phage Display of Peptides and Proteins, Academic Press, Inc., 1996) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.).

Another form of an anti-MAp19 antibody fragment useful in this aspect of the invention is a peptide coding for a single complementarity-determining region (CDR) that binds to an epitope on a MAp19 antigen and inhibits lectin-dependent complement activation. CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), Cambridge University Press, page 166, 1995); and Ward et al., “Genetic Manipulation and Expression of Antibodies,” in Monoclonal Antibodies: Principles and Applications, Birch et al. (eds.), Wiley-Liss, Inc., page 137, 1995).

The MAp19 antibodies described herein are administered to a subject in need thereof to inhibit lectin-dependent complement activation. In some embodiments, the MAp19 inhibitory agent is a high-affinity human or humanized monoclonal anti-MAp19 antibody with reduced effector function. In some embodiments, the MAp19 inhibitory agent is an anti-MAp19 antibody that does not bind to MASP-2.

### Peptide Inhibitors

In some embodiments of this aspect of the invention, the MAp19 inhibitory agent comprises isolated MAp19 peptide inhibitors, including isolated natural peptide inhibitors and synthetic peptide inhibitors that inhibit the lectin-dependent complement activation system. As used herein, the term “isolated MAp19 peptide inhibitors” refers to peptides that bind to or interact with MAp19 and inhibit lectin-dependent complement activation that are substantially pure and are essentially free of other substances with which they may be found in nature to an extent practical and appropriate for their intended use. In some embodiments, the isolated MAp19 peptide inhibitors do not bind to MASP-2.

Peptide inhibitors have been used successfully in vivo to interfere with protein-protein interactions and catalytic sites. For example, peptide inhibitors to adhesion molecules structurally related to LFA-1 have recently been approved for clinical use in coagulopathies (Ohman, E. M., et al., Eur. Heart J. 16:50-55, 1995). Short linear peptides (<30 amino acids) have been described that prevent or interfere with integrin-dependent adhesion (Murayama, O., et al., J. Biochem. 120:445-51, 1996). Longer peptides, ranging in length from 25 to 200 amino acid residues, have also been used successfully to block integrin-dependent adhesion (Zhang, L., et al., J. Biol. Chem. 271(47):29953-57, 1996). In general, longer peptide inhibitors have higher affinities and/or slower off-rates than short peptides and may therefore be more potent inhibitors. Cyclic peptide inhibitors have also been shown to be effective inhibitors of integrins in vivo for the treatment of human inflammatory disease (Jackson, D. Y., et al., J. Med. Chem. 40:3359-68, 1997). One method of producing cyclic peptides involves the synthesis of peptides in which the terminal amino acids of the peptide are cysteines, thereby allowing the peptide to exist in a cyclic form by disulfide bonding between the terminal amino acids, which has been shown to improve affinity and half-life in vivo for the treatment of hematopoietic neoplasms (e.g., U.S. Pat. No. 6,649,592 to Larson).

### Synthetic MAp19 Peptide Inhibitors

MAp19 inhibitory peptides useful in the methods of this aspect of the invention are exemplified by amino acid sequences that mimic the target regions important for MAp19 function. The inhibitory peptides useful in the practice of the methods of the invention range in size from about 5 amino acids to about 300 amino acids. TABLE 3 provides a list of exemplary inhibitory peptides that may be useful in the practice of this aspect of the present invention. A candidate MAp19 inhibitory peptide may be tested for the ability to function as a MAp19 inhibitory agent in one of several assays including, for example, a lectin specific C4 cleavage assay (described in Example 2), and a C3b deposition assay (described in Example 4).

In some embodiments, the MAp19 inhibitory peptides are derived from MAp19 polypeptides and are selected from the full length mature MAp19 protein (SEQ ID NO:3), or from a particular domain of the MAp19 protein such as, for example, the CUBI domain (SEQ ID NO:8), or the region involved in MAp19 dimerization (SEQ ID NO:10). In particular, the peptide sequence DTERAPGKDTFYSLGSSLDITFRSDYSNEKPFTGF (SEQ ID NO:11) in the CUBI domain of MAp19 has been shown to be involved in binding to MBL (Stengaard-Pedersen, K., et al., 2003, supra; Gregory et al., 2004, supra). In addition, inhibitory peptides useful in the practice of the invention may comprise at least one of the side chains of Tyr59, Glu83, Tyr106 and Glu109 (in reference to SEQ ID NO:3) which were shown in the Gregory et al. crystallographic study to be available to mediate protein-to-protein interactions with MBL or L-ficolin. MAp19 inhibitory peptides may also be derived from MASP-2 (SEQ ID NO:6), or from MASP-1 (SEQ ID NO:43).

In some embodiments, MAp19 inhibitory peptides are derived from the lectin proteins that bind to MAp19 and are involved in the lectin complement pathway. Three different lectins have been identified that are involved in this pathway, including mannan-binding lectin (MBL), L-ficolin and H-ficolin. (Ikeda, K., et al., J. Biol. Chem. 262:7451-7454, 1987; Matsushita, M., et al., J. Exp. Med. 176:1497-2284, 2000; and Matsushita, M., et al., J. Immunol. 168:3502-3506, 2002). These lectins are present in serum as oligomers of homotrimeric subunits, each having N-terminal collagen-like fibers with carbohydrate recognition domains. These three different lectins have been shown to bind to MAp19. H-ficolin has an amino-terminal region of 24 amino acids, a collagen-like domain with 11 Gly-Xaa-Yaa repeats, a neck domain of 12 amino acids, and a fibrinogen-like domain of 207 amino acids (Matsushita, M., et al., J. Immunol. 168:3502-3506, 2002). H-ficolin binds to GlcNAc and agglutinates human erythrocytes coated with LPS derived from S. typhimurium, S. minnesota and E. coli. H-ficolin has been shown to be associated with MAp19 and MASP-2 and activates the lectin pathway. Id. L-ficolin/P35 also binds to GlcNAc and has been shown to be associated with MASP-2 and MAp19 in human serum and this complex has been shown to activate the lectin pathway (Matsushita, M., et al., J. Immunol. 164:2281, 2000). Accordingly, MAp19 inhibitory peptides useful in the present invention may comprise a region of at least 5 amino acids selected from the MBL protein (SEQ ID NO:14), the MASP-1 protein (SEQ ID NO:43) the H-ficolin protein (Genbank accession number NM_{—}173452), the M-ficolin protein (Genbank accession number O00602), and the L-ficolin protein (Genbank accession number NM_{—}015838).

More specifically, scientists have identified the MAp19 binding site on MBL to be within the 12 Gly-X-Y triplets “GKD GRD GTK GEK GEP GQG LRG LQG POG KLG POG NOG PSG SOG PKG QKG DOG KS” (SEQ ID NO:19) that lie between the hinge and the neck in the C-terminal portion of the collagen-like domain of MBP (Wallis, R., et al., J. Biol. Chem. 279:14065, 2004). This MAp19 binding site region is also highly conserved in human H-ficolin and human L-ficolin. A consensus binding site has been described that is present in all three lectin proteins comprising the amino acid sequence “OGK-X-GP” (SEQ ID NO:15) where the letter “O” represents hydroxyproline and the letter “X” is a hydrophobic residue (Wallis et al., supra). Accordingly, in some embodiments, MAp19 inhibitory peptides useful in this aspect of the invention are at least 6 amino acids in length and comprise SEQ ID NO:15. Peptides derived from MBL that include the amino acid sequence “GLR GLQ GPO GKL GPO G” (SEQ ID NO:17) have been shown to bind the CUBI region of MASP-2 in vitro which is also present in the MAp19 protein (Wallis et al., 2004, supra). To enhance binding to MAp19, peptides can be synthesized that are flanked by two GPO triplets at each end (“GPO GPO GLR GLQ GPO GKL GPO GPO GPO” SEQ ID NO:18) to enhance the formation of triple helices as found in the native MBL protein (as further described in Wallis, R., et al., J. Biol. Chem. 279:14065, 2004).

MAp19 inhibitory peptides may also be derived from human H-ficolin that include the sequence “GAO GSO GEK GAO GPQ GPO GPO GKM GPK GEO GDO” (SEQ ID NO:20) from the consensus MAp19 binding region in H-ficolin. Also included are peptides derived from human L-ficolin that include the sequence “GCO GLO GAO GDK GEA GTN GKR GER GPO GPO GKA GPO GPN GAO GEO” (SEQ ID NO:21) from the consensus MAp19 binding region in L-ficolin.

TABLE 3
EXEMPLARY MAP19 INHIBITORY PEPTIDES

| | |
|---|---|
| Sequence | Source |

| | |
|---|---|
| SEQ ID NO:6 | Human MASP-2 protein |
| | (mature) |
| SEQ ID NO:3 | Human MAp19 protein |
| | (mature) |
| SEQ ID NO:8 | CUBI domain of MAp19 |
| (aa 1-121 of SEQ ID NO:3) | |
| SEQ ID NO:10 | Dimerization region |
| TAPPGYRLRLYFTHFDLELSHLC | in MAp19 |
| EYDFVKLSSGAKVLATLCGQ | |
| SEQ ID NO:11 | MBL binding in MAp19 |
| DTERAPGKDTFYSLGSSLDITFR | CUBI domain |
| SDYSNEKPFTGF | |
| SEQ ID NO:14 | Human MBL protein |
| SEQ ID NO:15 | Synthetic peptide |
| OGK-X-GP, | Consensus binding site |
| Where “O” = hydroxyproline | from Human MBL and |
| and “X” is a hydrophobic | Human ficolins |
| amino acid residue | |
| SEQ ID NO:16 | Human MBL core binding |
| OGKLG | site |
| SEQ ID NO:17 | Human MBP Triplets 6-10- |
| GLR GLQ GPO GKL GPO G | demonstrated binding to |
| | MASP-2 |
| SEQ ID NO:18 | Human MBP Triplets with |
| GPOGPOGLRGLQGPOGKLGPOG | GPO added to enhance |
| POGPO | formation of triple |
| | helices |
| SEQ ID NO:19 | Human MBP Triplets 1-17 |
| GKDGRDGTKGEKGEPGQGLRG | |
| LQGPOGKLGPOGNOGPSGSOGP | |
| KGQKGDOGKS | |
| SEQ ID NO:20 | Human H-Ficolin (Hataka) |
| GAOGSOGEKGAOGPQGPOGPO | derived peptide |
| GKMGPKGEOGDO | |
| SEQ ID NO:21 | Human Ficolin P35 |
| GCOGLOGAOGDKGEAGTNGKR | derived peptide |
| GERGPOGPOGKAGPOGPNGAO | |
| GEC | |
| SEQ ID NO:43 | Human MASP-1 protein |

Note: The letter “O” represents hydroxyproline. The letter “X” is a hydrophobic residue.

### Natural Peptide Inhibitors

In addition to the inhibitory peptides described above, MAp19 inhibitory peptides useful in the method of the invention include peptides containing the MAp19-binding CDR3 region of anti-MAp19 MoAb obtained as described herein. The sequence of the CDR regions for use in synthesizing the peptides may be determined by methods known in the art. The heavy chain variable region is a peptide which generally ranges from 100 to 150 amino acids in length. The light chain variable region is a peptide which generally ranges from 80 to 130 amino acids in length. The CDR sequences within the heavy and light chain variable regions include only approximately 3-25 amino acid sequences which may be easily sequenced by one of ordinary skill in the art.

Those skilled in the art will recognize that substantially homologous variations of the MAp19 inhibitory peptides described above will also exhibit MAp19 inhibitory activity. Exemplary variations include, but are not necessarily limited to, peptides having insertions, deletions, replacements, and/or additional amino acids on the carboxy-terminus or amino-terminus portions of the subject peptides and mixtures thereof. Accordingly, those homologous peptides having MAp19 inhibitory activity are considered to be useful in the methods of this invention. The peptides described may also include duplicating motifs and other modifications with conservative substitutions. Conservative variants are described elsewhere herein, and include the exchange of an amino acid for another of like charge, size, or hydrophobicity and the like.

MAp19 inhibitory peptides may be modified to increase solubility and/or to maximize the positive or negative charge in order to more closely resemble the segment in the intact protein. The derivative may or may not have the exact primary amino acid structure of a peptide disclosed herein so long as the derivative functionally retains the desired property of MAp19 inhibition. The modifications can include amino acid substitution with one of the commonly known twenty amino acids or with another amino acid, with a derivatized or substituted amino acid with ancillary desirable characteristics, such as resistance to enzymatic degradation or with a D-amino acid or substitution with another molecule or compound, such as a carbohydrate, which mimics the natural confirmation and function of the amino acid, amino acids or peptide; amino acid deletion; amino acid insertion with one of the commonly known twenty amino acids or with another amino acid, with a derivatized or substituted amino acid with ancillary desirable characteristics, such as resistance to enzymatic degradation or with a D-amino acid or substitution with another molecule or compound, such as a carbohydrate, which mimics the natural confirmation and function of the amino acid, amino acids or peptide; or substitution with another molecule or compound, such as a carbohydrate or nucleic acid monomer, which mimics the natural conformation, charge distribution and function of the parent peptide. Peptides may also be modified by acetylation or amidation.

The synthesis of derivative inhibitory peptides can rely on known techniques of peptide biosynthesis, carbohydrate biosynthesis and the like. As a starting point, the artisan may rely on a suitable computer program to determine the conformation of a peptide of interest. Once the conformation of peptide disclosed herein is known, then the artisan can determine in a rational design fashion what sort of substitutions can be made at one or more sites to fashion a derivative that retains the basic conformation and charge distribution of the parent peptide but which may possess characteristics which are not present or are enhanced over those found in the parent peptide. Once candidate derivative molecules are identified, the derivatives can be tested to determine if they function as MAp19 inhibitory agents using the assays described herein.

### Screening for MAp19 Inhibitory Peptides

One may also use molecular modeling and rational molecular design to generate and screen for peptides that mimic the molecular structures of key binding regions of MAp19 and inhibit the complement activities of MAp19. The molecular structures used for modeling include the CDR regions of anti-MAp19 monoclonal antibodies, as well as the target regions known to be important for MAp19 function including the region required for dimerization, the region involved in binding to MBL, and the binding residues identified in the X-ray crystallographic study by Gregory et al. previously described. Methods for identifying peptides that bind to a particular target are well known in the art. For example, molecular imprinting may be used for the de novo construction of macromolecular structures such as peptides which bind to a particular molecule. See, for example, Shea, K. J., “Molecular Imprinting of Synthetic Network Polymers: The de Novo synthesis of Macromolecular Binding and Catalytic Sties,” TRIP 2(5), May 1994. As an illustrative example, one method of preparing mimics of MAp19 binding peptides is as follows. Functional monomers of a known MAp19 binding peptide or the binding region of an anti-MAp19 antibody that exhibits MAp19 inhibition (the template) are polymerized. The template is then removed, followed by polymerization of a second class of monomers in the void left by the template, to provide a new molecule that exhibits one or more desired properties that are similar to the template. In addition to preparing peptides in this manner, other MAp19 binding molecules that are MAp19 inhibitory agents such as polysaccharides, nucleosides, drugs, nucleoproteins, lipoproteins, carbohydrates, glycoproteins, steroid, lipids and other biologically active materials can also be prepared. This method is useful for designing a wide variety of biological mimics that are more stable than their natural counterparts because they are typically prepared by free radical polymerization of function monomers, resulting in a compound with a nonbiodegradable backbone.

### Peptide Synthesis

The MAp19 inhibitory peptides can be prepared using techniques well known in the art, such as the solid-phase synthetic technique initially described by Merrifield in J. Amer. Chem. Soc. 85:2149-2154, 1963. Automated synthesis may be achieved, for example, using Applied Biosystems 431A Peptide Synthesizer (Foster City, Calif.) in accordance with the instructions provided by the manufacturer. Other techniques may be found, for example, in Bodanszky, M., et al., Peptide Synthesis, second edition, John Wiley & Sons, 1976, as well as in other reference works known to those skilled in the art.

The peptides can also be prepared using standard genetic engineering techniques known to those skilled in the art. For example, the peptide can be produced enzymatically by inserting nucleic acid encoding the peptide into an expression vector, expressing the DNA, and translating the DNA into the peptide in the presence of the required amino acids. The peptide is then purified using chromatographic or electrophoretic techniques, or by means of a carrier protein that can be fused to, and subsequently cleaved from, the peptide by inserting into the expression vector in phase with the peptide encoding sequence a nucleic acid sequence encoding the carrier protein. The fusion protein-peptide may be isolated using chromatographic, electrophoretic or immunological techniques (such as binding to a resin via an antibody to the carrier protein). The peptide can be cleaved using chemical methodology or enzymatically, as by, for example, hydrolases.

The MAp19 inhibitory peptides that are useful in the method of the invention can also be produced in recombinant host cells following conventional techniques. To express a MAp19 inhibitory peptide encoding sequence, a nucleic acid molecule encoding the peptide must be operably linked to regulatory sequences that control transcriptional expression in an expression vector and then introduced into a host cell. In addition to transcriptional regulatory sequences, such as promoters and enhancers, expression vectors can include translational regulatory sequences and a marker gene, which is suitable for selection of cells that carry the expression vector.

Nucleic acid molecules that encode a MAp19 inhibitory peptide can be synthesized with “gene machines” using protocols such as the phosphoramidite method. If chemically synthesized double-stranded DNA is required for an application such as the synthesis of a gene or a gene fragment, then each complementary strand is made separately. The production of short genes (60 to 80 base pairs) is technically straightforward and can be accomplished by synthesizing the complementary strands and then annealing them. For the production of longer genes, synthetic genes (double-stranded) are assembled in modular form from single-stranded fragments that are from 20 to 100 nucleotides in length. For reviews on polynucleotide synthesis, see, for example, Glick and Pasternak, Molecular Biotechnology, Principles and Applications of Recombinant DNA (ASM Press, 1994); Itakura, K., et al., Annu. Rev. Biochem. 53:323, 1984; and Climie, S., et al., Proc. Nat'l Acad. Sci. USA 87:633, 1990.

### Small Molecule Inhibitors

In some embodiments, MAp19 inhibitory agents are small molecule inhibitors including natural and synthetic substances that have a low molecular weight, such as for example, peptides, peptidomimetics and nonpeptide inhibitors (including oligonucleotides and organic compounds). Small molecule inhibitors of MAp19 can be generated based on the molecular structure of the variable regions of the anti-MAp19 antibodies.

Small molecule inhibitors may also be designed and generated based on the MAp19 crystal structure using computational drug design (Kuntz, I. D., et al., Science 257:1078 1992). The crystal structure of human MAp19 has been described (Gregory, L. A., et al., J. Biol. Chem., 2004, supra). Using the method described by Kuntz et al., the MAp19 crystal structure coordinates are used as an input for a computer program such as DOCK, which outputs a list of small molecule structures that are expected to bind to MAp19. Use of such computer programs is well known to one of skill in the art. For example, the crystal structure of the HIV-1 protease inhibitor was used to identify unique nonpeptide ligands that are HIV-1 protease inhibitors by evaluating the fit of compounds found in the Cambridge Crystallographic database to the binding site of the enzyme using the program DOCK (Kuntz, I. D., et al., J. Mol. Biol. 161:269-288, 1982; DesJarlais, R. L., et al., PNAS 87:6644-6648 1990).

The list of small molecule structures that are identified by a computational method as potential MAp19 inhibitors are screened using a MAp19 binding assay such as described in Example 9. The small molecules that are found to bind to MAp19 are then assayed in a functional assay such as described in Example 2 to determine if they inhibit lectin-dependent complement activation.

### MAp19 Soluble Receptors

Other suitable MAp19 inhibitory agents are believed to include MAp19 soluble receptors, which may be produced using techniques known to those of ordinary skill in the art.

### Expression Inhibitors of MAp19

In another embodiment of this aspect of the invention, the MAp19 inhibitory agent is a MAp19 expression inhibitor capable of inhibiting lectin-dependent complement activation. In the practice of this aspect of the invention, representative MAp19 expression inhibitors include MAp19 antisense nucleic acid molecules (such as antisense mRNA, antisense DNA or antisense oligonucleotides), MAp19 ribozymes and MAp19 RNAi molecules.

Antisense RNA and DNA molecules act to directly block the translation of MAp19 and/or MASP-2 mRNA by hybridizing to MAp19 mRNA and preventing alternative splicing and/or translation of MAp19 and/or MASP-2 protein. An antisense nucleic acid molecule may be constructed in a number of different ways provided that it is capable of binding to MAp19 sequences and interfering with the protein expression of MAp19 and/or MASP-2. For example, an antisense nucleic acid molecule can be constructed by inverting the coding region (or a portion thereof) of MAp19 cDNA (SEQ ID NO:1) relative to its normal orientation for transcription to allow for the transcription of its complement.

The antisense nucleic acid molecule is usually substantially identical to at least a portion of the target gene or genes. The nucleic acid, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 8 or so nucleotides may be used as the antisense nucleic acid molecule, although a longer sequence is preferable. In the present invention, a representative example of a useful inhibitory agent of MAp19 is an antisense MAp19 nucleic acid molecule which is at least ninety percent identical to the complement of the MAp19 cDNA consisting of the nucleic acid sequence set forth in SEQ ID NO:1. The nucleic acid sequence set forth in SEQ ID NO:1 encodes the MAp19 protein consisting of the amino acid sequence set forth in SEQ ID NO:2.

The targeting of antisense oligonucleotides to bind MAp19 mRNA is another mechanism that may be used to reduce the level of MAp19 and/or MASP-2 protein synthesis. For example, the synthesis of polygalacturonase and the muscarine type 2 acetylcholine receptor are inhibited by antisense oligonucleotides directed to their respective mRNA sequences (U.S. Pat. No. 5,739,119 to Cheng and U.S. Pat. No. 5,759,829 to Shewmaker). Furthermore, examples of antisense inhibition have been demonstrated with the nuclear protein cyclin, the multiple drug resistance gene (MDG1), ICAM-1, E-selectin, STK-1, striatal GABA_{A} receptor and human EGF (see, e.g., U.S. Pat. No. 5,801,154 to Baracchini; U.S. Pat. No. 5,789,573 to Baker; U.S. Pat. No. 5,718,709 to Considine and U.S. Pat. No. 5,610,288 to Rubenstein).

A system has been described that allows one of ordinary skill to determine which oligonucleotides are useful in the invention, which involves probing for suitable sites in the target mRNA using Rnase H cleavage as an indicator for accessibility of sequences within the transcripts. Scherr, M., et al., Nucleic Acids Research 26:5079-5085, 1998; Lloyd et al., Nucleic Acids Res. 29:3665-3673, 2001. A mixture of antisense oligonucleotides that are complementary to certain regions of the MAp19 transcript is added to cell extracts expressing MAp19, such as hepatocytes, and hybridized in order to create an RNAseH vulnerable site. This method can be combined with computer-assisted sequence selection that can predict optimal sequence selection for antisense compositions based upon their relative ability to form dimers, hairpins or other secondary structures that would reduce or prohibit specific binding to the target mRNA in a host cell. These secondary structure analysis and target site selection considerations may be performed using the OLIGO primer analysis software (Rychlik, I., 1997) and the BLASTN 2.0.5 algorithm software (Altschul, S. F., et al., Nucl. Acids Res. 25:3389-3402, 1997). The antisense compounds directed towards the target sequence preferably comprise from about 8 to about 50 nucleotides in length. Antisense oligonucleotides comprising from about 9 to about 35 or so nucleotides are particularly preferred. The inventors contemplate all oligonucleotide compositions in the range of 9 to 35 nucleotides (i.e., those of 9, 10, 11, 12, 13, 14, 15, 16, 17, 18, 19, 20, 21, 22, 23, 24, 25, 26, 27, 28, 29, 30, 31, 32, 33, 34 or 35 or so bases in length) are highly preferred for the practice of antisense oligonucleotide-based methods of the invention. Highly preferred target regions of the MAp19 mRNA are those that are at or near the AUG translation initiation codon, and those sequences that are substantially complementary to 5′ regions of the mRNA, e.g., between the −10 and +10 regions of the MAp19 gene nucleotide sequence (SEQ ID NO:1). Exemplary MAp19 expression inhibitors are provided in TABLE 4.

The term “oligonucleotide” as used herein, refers to an oligomer or polymer of ribonucleic acid (RNA) or deoxyribonucleic acid (DNA) or mimetics thereof. This term also covers those oligonucleobases composed of naturally occurring nucleotides, sugars and covalent internucleoside (backbone) linkages as well as oligonucleotides having non-naturally-occurring modifications. These modifications allow one to introduce certain desirable properties that are not offered through naturally occurring oligonucleotides, such as reduced toxic properties, increased stability against nuclease degradation and enhanced cellular uptake. In illustrative embodiments, the antisense compounds of the invention differ from native DNA by the modification of the phosphodiester backbone to extend the life of the antisense oligonucleotide, in which the phosphate substituents are replaced by phosphorothioates. Likewise, one or both ends of the oligonucleotide may be substituted by one or more acridine derivatives that intercalate between adjacent basepairs within a strand of nucleic acid.

Another alternative to antisense is the use of “RNA interference” (RNAi). Double-stranded RNAs (dsRNAs) can provoke gene silencing in mammals in vivo. The natural function of RNAi and co-suppression appears to be protection of the genome against invasion by mobile genetic elements such as retrotransposons and viruses that produce aberrant RNA or dsRNA in the host cell when they become active (see, e.g., Jensen, J., et al., Nat. Genet. 21(2):209-12, 1999). The double-stranded RNA molecule may be prepared by synthesizing two RNA strands capable of forming a double-stranded RNA molecule, each having a length from about 19 to 25 (e.g., 19-23 nucleotides).

RNAi has been used to specifically inhibit proteins encoded by alternatively spliced mRNAs by using dsRNAs corresponding to specific alternative exons (Celotto, A. M., RNA 8:718-724, 2002). For example, an RNA sequence comprising SEQ ID NO:26 and SEQ ID NO 27 can be combined to form a dsRNA to inhibit the MAp19 specific exon 5. Additional dsRNA molecules useful in the methods of the invention may comprise the RNA corresponding to a sequence and its complement listed in TABLE 4. Preferably, at least one strand of RNA has a 3′ overhang from 1-5 nucleotides. The synthesized RNA strands are combined under conditions that form a double-stranded molecule. The RNA sequence may comprise at least an 8 nucleotide portion of SEQ ID NO:1 with a total length of 25 nucleotides or less. The design of siRNA sequences for a given target is within the ordinary skill of one in the art. Commercial services are available that design siRNA sequence and guarantee at least 70% knockdown of expression (Qiagen, Valencia, Calif.).

The dsRNA may be administered as a pharmaceutical composition and carried out by known methods, wherein a nucleic acid is introduced into a desired target cell. Commonly used gene transfer methods include calcium phosphate, DEAE-dextran, electroporation, microinjection and viral methods. Such methods are taught in Ausubel et al., Current Protocols in Molecular Biology, John Wiley & Sons, Inc., 1993.

Ribozymes can also be utilized to decrease the amount and/or biological activity of MAp19, such as ribozymes which target MAp19 mRNA. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region of at least about nine nucleotides, which is complementary in nucleotide sequence to at least part of the target MAp19 mRNA, and a catalytic region that is adapted to cleave the target MAp19 mRNA (see generally, EPA No. 0 321 201; WO88/04300; Haseloff, J., Nature 334:585-591, 1988; Fedor, M. J., Proc. Natl. Acad. Sci. USA 87:1668-1672, 1990; Cech, T. R., Ann. Rev. Biochem. 55:599-629, 1986).

Ribozymes can either be targeted directly to cells in the form of RNA oligonucleotides incorporating ribozyme sequences, or introduced into the cell as an expression vector encoding the desired ribozymal RNA. Ribozymes may be used and applied in much the same way as described for antisense polynucleotides.

Antisense RNA and DNA, ribozymes and RNAi molecules useful in the methods of the invention may be prepared by any method known in the art for the synthesis of DNA and RNA molecules. These include techniques for chemically synthesizing oligodeoxyribonucleotides and oligoribonucleotides well known in the art, such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well known modifications of the DNA molecules may be introduced as a means of increasing stability and half-life. Useful modifications include, but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

TABLE 4
EXEMPLARY EXPRESSION INHIBITORS OF MAP19

| | |
|---|---|
| SEQ ID NO:22 | Nucleic acid sequence |
| | encoding MAp19 CUBI |
| | domain (sense) |
| SEQ ID NO:23 | Nucleotides 27-50 of |
| 5′ ATGAGGCTGCTGACCCTC | SEQ ID NO:1 comprising MAp19 |
| CTGGGC 3′ | translation start site- |
| | (sense) |
| SEQ ID NO:24 | Nucleotides 72-98 of |
| 5′ ACCCCCTTGGGCCCGAAG | SEQ ID NO:1 comprising region |
| TGGCCTGAA 3′ | encoding MAp19 N-terminal |
| | extension (sense) |
| SEQ ID NO:25 | Nucleotides 366-401 of |
| 5′ GACATTACCTTCCGCTCC | SEQ ID NO:1 comprising the |
| GACTCCAACGAGAAG 3′ | region encoding MAp19 MBL |
| | binding site (sense) |
| SEQ ID NO:26 | MAp19 Exon 5 encoding EQSL |
| 5′ GAGCAGAGCCTCTAG 3′ | sense |
| SEQ ID NO:27 | MAp19 Exon 5 encoding EQSL |
| 5′ CTAGAGGCTCTGCTC 3′ | antisense |

V. MAp19 Specific Agents that do not Bind to MASP-2

In another aspect, the present invention provides MAp19 specific agents that do not bind to MASP-2. As used herein a “MAp19 specific agent that does not bind to MASP-2” is any agent that binds to or interacts with MAp19 including anti-MAp19 antibodies and MAp19 binding fragments thereof, natural and synthetic peptides, small molecules, expression inhibitors and isolated natural inhibitors but that does not directly bind to MASP-2, but may regulate MASP-2 or the function thereof. In some embodiments, the MAp19 specific agent is an inhibitory agent. In further embodiments, the MAp19 specific inhibitory agent has reduced effector function. In some embodiments, the MAp19 inhibitory agent is a MAp19 inhibitory peptide or a non-peptide MAp19 inhibitor. The MAp19 inhibitory agents described herein can be screened to determine if they bind to MASP-2 in a suitable assay such as a binding assay described in Example 9.

In some embodiments the MAp19 specific agent is an anti-MAp19 antibody or fragments thereof. The methods for obtaining MAp19 specific agents, such as anti-MAp19 antibodies that do not bind to MASP-2, are provided herein. MAp19 specific agents that do not bind to MASP-2 are useful for diagnostic purposes, such as for determining serum levels of MAp19 in patient samples.

In some embodiments the MAp19 specific agent is a MAp19 specific inhibitory agent. Once identified, a MAp19 specific agent can be tested to determine if it functions as a MAp19 inhibitory agent by inhibiting the lectin-dependent pathway of complement activation using the assays described herein.

VI. Method of Producing Anti-MAp19 Antibodies that do not Bind to MASP-2

In another aspect, the present invention provides methods of producing anti-human MAp19 specific antibodies that do not bind to human MASP-2. The method comprises the steps of generating a MASP-2−/− transgenic animal (e.g., as described in Example 3), integrating a human MASP-2 transgene into said animal (e.g., as described in Example 6), introducing a human MAp19 derived antigen into said animal (e.g., as described in Example 9) and selecting antibodies that specifically bind to human MAp19 that do not bind to human MASP-2 (e.g., as described in Example 10). In one embodiment, the MASP-2−/− transgenic animal is also MAp19−/− (e.g., as described in Example 10). In some embodiments of this aspect of the invention, MAp19 specific antibodies produced by the methods of the invention are provided. In some embodiments the MAp19 specific antibodies produced by the method of the invention are MAp19 inhibitory agents. The MAp19 specific antibodies are useful for specifically detecting MAp19 in vivo and may be used, for example, in the diagnosis of MAp19 associated diseases and conditions. The MAp19 specific antibodies that are MAp19 inhibitory agents are useful in the therapeutic methods of the invention.

VII. Methods of Increasing the Expression of MAp19 in a Subject in Need Thereof

In another aspect of the invention, MAp19 protein compositions, medicaments and methods for using the same are provided for the treatment of MAp19 deficiency disorders.

The amount and/or biological activity of MAp19 in a subject can be increased by any suitable method, such as one or more of the following, representative methods: the delivery of nucleic acid molecules encoding MAp19 into a living subject; increasing the level of endogenous MAp19 transcription and/or translation within the body of a subject; delivery of MAp19 protein (or MAp19 fragments that retain the ability to modulate MASP-2-dependent complement activation) into the body of a living subject; or attaching to the body of a living subject a structure comprising MAp19, or MAp19 peptides retaining the ability to modulate MASP-2-dependent complement activation.

Any MAp19 protein that improves complement activation is useful in the practice of the present invention. MAp19 proteins useful in the methods of the present invention include naturally purified MAp19 protein (which may be chemically modified after purification), chemically synthesized MAp19 protein, and MAp19 protein produced by recombinant techniques from a prokaryotic or eukaryotic host, including, for example, bacterial, yeast, insect, mammalian, avian and higher plant cells.

In some embodiments, the invention provides pharmaceutical compositions comprising a therapeutically effective amount of MAp19 proteins in combination with a pharmaceutically acceptable carrier or vehicle. A therapeutically effective amount of MAp19 is an amount sufficient to produce a clinically significant change in the treated condition, such as a clinically significant change in complement activation. In general, pharmaceutical formulations will include a MAp19 polypeptide in combination with a pharmaceutically acceptable vehicle, such as saline, buffered saline, 5% dextrose in water, for topical or parenteral, particularly intravenous or subcutaneous delivery according to conventional methods. Formulations may further include one or more excipients, preservatives, solubilizers, buffering agents, and albumin to prevent protein loss on vial surfaces.

The amount and/or biological activity of MAp19 in an animal can be also be increased, for example, by delivery of nucleic acid molecules encoding MAp19, or a biologically active fragment thereof, into the body of an animal. By way of example, a vector that includes a nucleic acid molecule (typically a DNA molecule) that encodes an MAp19 protein can be introduced into any suitable host cell, including animal and human cells, and the encoded MAp19 protein expressed therein. The vector can be introduced into host cells in vitro, and the modified cells introduced into the body of an animal, or the vector can be introduced into cells, in vivo, within the body of an animal. Any art-recognized gene delivery method can be used to introduce a vector into one or more cells for expression therein, including: transduction, transfection, transformation, direct injection, electroporation, virus-mediated gene delivery, amino acid-mediated gene delivery, biolistic gene delivery, lipofection and heat shock. See, generally, Sambrook et al., supra. Representative, non-viral, methods of gene delivery into cells are disclosed in Huang, L., et al., Non-Viral Vectors for Gene Therapy, Academic Press, San Diego, Calif., 1999.

Expression vectors useful for expressing MAp19 protein, or biologically active fragments thereof, include chromosomal, episomal, and virus-derived vectors, e.g., vectors derived from bacterial plasmids, bacteriophages, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids. In certain embodiments in this regard, the vectors provide for specific expression, which may be inducible and/or cell type-specific. Among such expression vectors are those inducible by environmental factors that are easy to manipulate, such as temperature and nutrient additives.

Expression of MAp19 can be obtained using a vector containing the endogenous promoter elements of MAp19, as provided in SEQ ID NO:42. Such an expression cassette can be introduced into a body using a viral vector. For example, retroviruses are RNA viruses that have the ability to insert their genes into host cell chromosomes after infection. Retroviral vectors have been developed that lack the genes encoding viral proteins, but retain the ability to infect cells and insert their genes into the chromosomes of the target cell (Miller, A. D., Hum. Gen. Ther. 1:5-14, 1990). Adenoviral vectors are designed to be administered directly to patients. Unlike retroviral vectors, adenoviral vectors do not integrate into the chromosome of the host cell. Instead, genes introduced into cells using adenoviral vectors are maintained in the nucleus as an extrachromosomal element (episome) that persists for a limited time period. Adenoviral vectors will infect dividing and non-dividing cells in many different tissues in vivo including airway epithelial cells, endothelial cells, hepatocytes and various tumors (Trapnell, B. C., Adv. Drug Del. Rev. 12:185-199, 1993).

Another viral vector is the herpes simplex virus; a large, double-stranded DNA virus. Recombinant forms of the vaccinia virus can accommodate large inserts and are generated by homologous recombination. To date, this vector has been used to deliver, for example, interleukins (ILs), such as human IL-1β and the costimulatory molecules B7-1 and B7-2 (Peplinski, G. R., et al., Ann. Surg. Oncol. 2:151-9, 1995; Hodge, J. W., et al., Cancer Res. 54:5552-55, 1994).

A plasmid vector can be introduced into mammalian cells in a precipitate, such as a calcium phosphate precipitate, or in a complex with a charged lipid (e.g., LIPOFECTAMINE™; Life Technologies, Inc., Rockville, Md.) or in a complex with a virus (such as an adenovirus) or components of a virus (such as viral capsid peptides). If the vector is a virus, it may be packaged in vitro using an appropriate packaging cell line and then transduced into host cells.

For example, a vector may be formulated for delivery either encapsulated in a lipid particle, a liposome, a vesicle, or a gene activated collagen matrix. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

VIII. Pharmaceutical Compositions and Delivery Methods Dosing

In another aspect, the invention provides compositions for inhibiting the adverse effects of lectin-dependent complement activation comprising a therapeutically effective amount of a MAp19 inhibitory agent and a pharmaceutically acceptable carrier. The MAp19 inhibitory agents can be administered to a subject in need thereof, at therapeutically effective doses to treat or ameliorate conditions associated with lectin-dependent complement activation. A therapeutically effective dose refers to the amount of the MAp19 inhibitory agent sufficient to result in amelioration of symptoms of the condition.

Toxicity and therapeutic efficacy of MAp19 inhibitory agents can be determined by standard pharmaceutical procedures employing experimental animal models, such as the murine MAp19−/− mouse model expressing the human MAp19 transgene described in Example 5. Using such animal models, the NOAEL (no-observed adverse effect level) and the MED (the minimally effective dose) can be determined using standard methods. The dose ratio between NOAEL and MED effects is the therapeutic ratio, which is expressed as the ratio NOAEL/MED. MAp19 inhibitory agents that exhibit large therapeutic ratios or indices are most preferred. The data obtained from the cell culture assays and animal studies can be used in formulating a range of dosages for use in humans. The dosage of the MAp19 inhibitory agent preferably lies within a range of circulating concentrations that include the MED with little or no toxicity. The dosage may vary within this range depending upon the dosage form employed and the route of administration utilized.

For any compound formulation, the therapeutically effective dose can be estimated using animal models. For example, a dose may be formulated in an animal model to achieve a circulating plasma concentration range that includes the MED. Quantitative levels of the MAp19 inhibitory agent in plasma may also be measured, for example, by high performance liquid chromatography.

In addition to toxicity studies, effective dosage may also be estimated based on the amount of MAp19 protein present in a living subject and the binding affinity of the MAp19 inhibitory agent.

Generally, the dosage of administered compositions comprising MAp19 inhibitory agents varies depending on such factors as the subject's age, weight, height, sex, general medical condition, and previous medical history. As an illustration, MAp19 inhibitory agents, such as anti-MAp19 antibodies, can be administered in dosage ranges from about 0.010 to 10.0 mg/kg, preferably 0.010 to 1.0 mg/kg, more preferably 0.010 to 0.1 mg/kg of the subject body weight when delivered systemically.

Therapeutic efficacy of MAp19 inhibitory compositions for use in the methods of the present invention in a given subject, and appropriate dosages, can be determined in accordance with complement assays well known to those of skill in the art. Complement generates numerous specific products. During the last decade, sensitive and specific assays have been developed and are available commercially for most of these activation products, including the small activation fragments C3a, C4a, and C5a and the large activation fragments iC3b, C4d, Bb and sC5b-9. Most of these assays utilize monoclonal antibodies that react with new antigens (neoantigens) exposed on the fragment, but not on the native proteins from which they are formed, making these assays very simple and specific. Most rely on ELISA technology, although radioimmunoassay is still sometimes used for C3a and C5a. These latter assays measure both the unprocessed fragments and their ‘desArg’ fragments, which are the major forms found in the circulation. Unprocessed fragments and C5a_{desarg} are rapidly cleared by binding to cell surface receptors and are hence present in very low concentrations, whereas C3a_{desArg} does not bind to cells and accumulates in plasma. Measurement of C3a provides a sensitive, pathway-independent indicator of complement activation. Alternative pathway activation can be assessed by measuring the Bb fragment. Detection of the fluid-phase product of membrane attack pathway activation, sC5b-9, provides evidence that complement is being activated to completion. Because both the lectin and classical pathways generate the same activation products, C4a and C4d, measurement of these two fragments does not provide any information about which of these two pathways has generated the activation products.

Additional Agents

The compositions and methods comprising MAp19 inhibitory agents may optionally comprise one or more additional therapeutic agents, which may augment the activity of the MAp19 inhibitory agent or that provide related therapeutic functions in an additive or synergistic fashion. For example, one or more MAp19 inhibitory agents may be administered in combination with one or more anti-inflammatory and/or analgesic agents. The inclusion and selection of additional agent(s) will be determined to achieve a desired therapeutic result. Suitable anti-inflammatory and/or analgesic agents include: serotonin receptor antagonists; serotonin receptor agonists; histamine receptor antagonists; bradykinin receptor antagonists; kallikrein inhibitors; tachykinin receptor antagonists, including neurokinin₁ and neurokinin₂ receptor subtype antagonists; calcitonin gene-related peptide (CGRP) receptor antagonists; interleukin receptor antagonists; inhibitors of enzymes active in the synthetic pathway for arachidonic acid metabolites, including phospholipase inhibitors, including PLA₂ isoform inhibitors and PLC_{γ} isoform inhibitors, cyclooxygenase (COX) inhibitors (which may be either COX-1, COX-2 or nonselective COX-1 and -2 inhibitors), lipooxygenase inhibitors; prostanoid receptor antagonists including eicosanoid EP-1 and EP-4 receptor subtype antagonists and thromboxane receptor subtype antagonists; leukotriene receptor antagonists including leukotriene B₄ receptor subtype antagonists and leukotriene D₄ receptor subtype antagonists; opioid receptor agonists, including μ-opioid, δ-opioid, and κ-opioid receptor subtype agonists; purinoceptor agonists and antagonists including P_{2X} receptor antagonists and P_{2Y} receptor agonists; adenosine triphosphate (ATP)-sensitive potassium channel openers; MAP Kinase inhibitors; nicotinic acetylcholine inhibitors; and alpha adrenergic receptor agonists (including alpha-1, alpha-2 and nonselective alpha-1 and 2 agonists).

When used in the prevention or treatment of restenosis, the MAp19 inhibitory agent of the present invention may be combined with one or more anti-restenosis agents for concomitant administration. Suitable anti-restenosis agents include: antiplatelet agents including: thrombin inhibitors and receptor antagonists, adenosine diphosphate (ADP) receptor antagonists (also known as purinoceptor₁ receptor antagonists), thromboxane inhibitors and receptor antagonists and platelet membrane glycoprotein receptor antagonists; inhibitors of cell adhesion molecules, including selectin inhibitors and integrin inhibitors; anti-chemotactic agents; interleukin receptor antagonists; and intracellular signaling inhibitors including: protein kinase C (PKC) inhibitors and protein tyrosine phosphatases, modulators of intracellular protein tyrosine kinase inhibitors, inhibitors of src homology₂ (SH2) domains, and calcium channel antagonists.

The MAp19 inhibitory agents of the present invention may also be administered in combination with one or more other complement inhibitors. No complement inhibitors are currently approved for use in humans, however some pharmacological agents have been shown to block complement in vivo. Many of these agents are also toxic or are only partial inhibitors (Asghar, S. S., Pharmacol. Rev. 36:223-44, 1984), and use of these has been limited to use as research tools. K76COOH and nafamstat mesilate are two agents that have shown some effectiveness in animal models of transplantation (Miyagawa, S., et al., Transplant Proc. 24:483-484, 1992). Low molecular weight heparins have also been shown to be effective in regulating complement activity (Edens, R. E., et al., Complement Today pp. 96-120, Basel: Karger, 1993). It is believed that these small molecule inhibitors may be useful as agents to use in combination with the MAp19 inhibitory agents of the present invention.

Other naturally occurring complement inhibitors may be useful in combination with the MAp19 inhibitory agents of the present invention. Biological inhibitors of complement include soluble complement factor 1 (sCR1). This is a naturally occurring inhibitor that can be found on the outer membrane of human cells. Other membrane inhibitors include DAF, MCP and CD59. Recombinant forms have been tested for their anti-complement activity in vitro and in vivo. sCR1 has been shown to be effective in xenotransplantation, wherein the complement system (both alternative and classical) provides the trigger for a hyperactive rejection syndrome within minutes of perfusing blood through the newly transplanted organ (Platt, J. L., et al., Immunol Today 11:450-6, 1990; Marino, I. R., et al., Transplant Proc. 1071-6, 1990; Johnstone, P. S., et al., Transplantation 54:573-6, 1992). The use of sCR1 protects and extends the survival time of the transplanted organ, implicating the complement pathway in the pathogenesis of organ survival (Leventhal, J. R., et al., Transplantation 55:857-66, 1993; Pruitt, S. K., et al., Transplantation 57:363-70, 1994).

Suitable additional complement inhibitors for use in combination with the compositions of the present invention also include, by way of example, MoAbs such as those being developed by Alexion Pharmaceuticals, Inc., New Haven, Conn., and anti-properdin MoAbs.

When used in the treatment of arthritides (e.g., osteoarthritis and rheumatoid arthritis), the MAp19 inhibitory agent of the present invention may be coupled with one or more chondroprotective agents, which may include one or more promoters of cartilage anabolism and/or one or more inhibitors of cartilage catabolism, and suitably both an anabolic agent and a catabolic inhibitory agent. Suitable anabolic promoting chondroprotective agents include interleukin (IL) receptor agonists including IL-4, IL-10, IL-13, rhIL-4, rhIL-10 and rhIL-13, and chimeric IL-4, IL-10 or IL-13; Transforming growth factor-β superfamily agonists, including TGF-β, TGF-β1, TGF-β2, TGF-β3, bone morphogenic proteins including BMP-2, BMP-4, BMP-5, BMP-6, BMP-7 (OP-1), and OP-2/BMP-8, growth-differentiation factors including GDF-5, GDF-6 and GDF-7, recombinant TGF-βs and BMPs, and chimeric TGF-βs and BMPs; insulin-like growth factors including IGF-1; and fibroblast growth factors including bFGF. Suitable catabolic inhibitory chondroprotective agents include Interleukin-1 (IL-1) receptor antagonists (IL-1ra), including soluble human IL-1 receptors (shuIL-1R), rshuIL-1R, rhIL-1ra, anti-IL1-antibody, AF11567, and AF 12198; Tumor Necrosis Factor (TNF) Receptor Antagonists (TNF-α), including soluble receptors including sTNFR1 and sTNFR11, recombinant TNF soluble receptors, and chimeric TNF soluble receptors including chimeric rhTNFR:Fc, Fc fusion soluble receptors and anti-TNF antibodies; cyclooxygenase-2 (COX-2 specific) inhibitors, including DuP 697, SC-58451, celecoxib, rofecoxib, nimesulide, diclofenac, meloxicam, piroxicam, NS-398, RS-57067, SC-57666, SC-58125, flosulide, etodolac, L-745,337 and DFU-T-614; Mitogen-activated protein kinase (MAPK) inhibitors, including inhibitors of ERK1, ERK2, SAPK1, SPAK2a, SAPK2b, SAPK2d, SAPK3, including SB 203580, SB 203580 iodo, SB202190, SB 242235, SB 220025, RWJ 67657, RWJ 68354, FR 133605, L-167307, PD 98059, PD 169316; inhibitors of nuclear factor kappa B (NFκB), including caffeic acid phenylethyl ester (CAPE), DM-CAPE, SN-50 peptide, hymenialdisine and pyrolidone dithiocarbamate; nitric oxide synthase (NOS) inhibitors, including N^{G}-monomethyl-L-arginine, 1400W, diphenyleneiodium, S-methyl isothiourea, S-(aminoethyl) isothiourea, L-N⁶-(1-iminoethyl)lysine, 1,3-PBITU, 2-ethyl-2-thiopseudourea, aminoguanidine, N^{ω}-nitro-L-arginine, and N^{ω}-nitro-L-arginine methyl ester, inhibitors of matrix metalloproteinases (MMPs), including inhibitors of MMP-1, MMP-2, MMP-3, MMP-7, MMP-8, MMP-9, MMP-10, MMP-11, MMP-12, MMP-13, MMP-14 and MMP-15, and including U-24522, minocycline, 4-Abz-Gly-Pro-D-Leu-D-Ala-NHOH, Ac-Arg-Cys-Gly-Val-Pro-Asp-NH₂, rhuman TIMP1, rhuman TIMP2, and phosphoramidon; cell adhesion molecules, including integrin agonists and antagonists including αVβ3 MoAb LM 609 and echistatin; anti-chemotactic agents including F-Met-Leu-Phe receptors, IL-8 receptors, MCP-1 receptors and MIP1-I/RANTES receptors; intracellular signaling inhibitors, including (a) protein kinase inhibitors, including both (i) protein kinase C (PKC) inhibitors (isozyme) including calphostin C, G-6203 and GF 109203X and (ii) protein tyrosine kinase inhibitors, (b) modulators of intracellular protein tyrosine phosphatases (PTPases) and (c) inhibitors of SH2 domains (src Homology₂ domains).

For some applications, it may be beneficial to administer the MAp19 inhibitory agents of the present invention in combination with a spasm inhibitory agent. For example, for urogenital applications, it may be beneficial to include at least one smooth muscle spasm inhibitory agent and/or at least one anti-inflammation agent, and for vascular procedures it may be useful to include at least one vasospasm inhibitor and/or at least one anti-inflammation agent and/or at least one anti-restenosis agent. Suitable examples of spasm inhibitory agents include: serotonin₂ receptor subtype antagonists; tachykinin receptor antagonists; nitric oxide donors; ATP-sensitive potassium channel openers; calcium channel antagonists; and endothelin receptor antagonists.

### Pharmaceutical Carriers and Delivery Vehicles

In general, the MAp19 inhibitory agent compositions of the present invention, combined with any other selected therapeutic agents, are suitably contained in a pharmaceutically acceptable carrier. The carrier is non-toxic, biocompatible and is selected so as not to detrimentally affect the biological activity of the MAp19 inhibitory agent (and any other therapeutic agents combined therewith). Exemplary pharmaceutically acceptable carriers for peptides are described in U.S. Pat. No. 5,211,657 to Yamada. The anti-MAp19 antibodies and inhibitory peptides useful in the invention may be formulated into preparations in solid, semi-solid, gel, liquid or gaseous forms such as tablets, capsules, powders, granules, ointments, solutions, depositories, inhalants and injections allowing for oral, parenteral or surgical administration. The invention also contemplates local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting example, microparticles, microspheres, nanospheres or nanoparticles composed of proteins, liposomes, carbohydrates, synthetic organic compounds, inorganic compounds, polymeric or copolymeric hydrogels and polymeric micelles. Suitable hydrogel and micelle delivery systems include the PEO:PHB:PEO copolymers and copolymer/cyclodextrin complexes disclosed in WO 2004/009664 A2 and the PEO and PEO/cyclodextrin complexes disclosed in U.S. 2002/0019369 A1. Such hydrogels may be injected locally at the site of intended action, or subcutaneously or intramuscularly to form a sustained release depot.

For intra-articular delivery, the MAp19 inhibitory agent may be carried in above-described liquid or gel carriers that are injectable, above-described sustained release delivery vehicles that are injectable, or a hyaluronic acid or hyaluronic acid derivative.

For oral administration of non-peptidergic agents, the MAp19 inhibitory agent may be carried in an inert filler or diluent such as sucrose, cornstarch, or cellulose.

For topical administration, the MAp19 inhibitory agent may be carried in ointment, lotion, cream, gel, drop, suppository, spray, liquid or powder, or in gel or microcapsular delivery systems via a transdermal patch.

Various nasal and pulmonary delivery systems, including aerosols, metered-dose inhalers, dry powder inhalers, and nebulizers, are being developed and may suitably be adapted for delivery of the present invention in an aerosol, inhalant, or nebulized delivery vehicle, respectively.

For intrathecal (IT) or intracerebroventricular (ICV) delivery, appropriately sterile delivery systems (e.g., liquids; gels, suspensions, etc.) can be used to administer the present invention.

The compositions of the present invention may also include biocompatible excipients, such as dispersing or wetting agents, suspending agents, diluents, buffers, penetration enhancers, emulsifiers, binders, thickeners, flavouring agents (for oral administration).

### Pharmaceutical Carriers for Antibodies and Peptides

More specifically with respect to anti-MAp19 antibodies and inhibitory peptides, exemplary formulations can be parenterally administered as injectable dosages of a solution or suspension of the compound in a physiologically acceptable diluent with a pharmaceutical carrier that can be a sterile liquid such as water, oils, saline, glycerol or ethanol. Additionally, auxiliary substances such as wetting or emulsifying agents, surfactants, pH buffering substances and the like can be present in compositions comprising anti-MAp19 antibodies and inhibitory peptides. Additional components of pharmaceutical compositions include petroleum (such as of animal, vegetable or synthetic origin), for example, soybean oil and mineral oil. In general, glycols such as propylene glycol or polyethylene glycol are preferred liquid carriers for injectable solutions.

The anti-MAp19 antibodies and inhibitory peptides can also be administered in the form of a depot injection or implant preparation that can be formulated in such a manner as to permit a sustained or pulsatile release of the active agents.

Pharmaceutically Acceptable Carriers for Expression Inhibitors

More specifically with respect to expression inhibitors useful in the methods of the invention, compositions are provided that comprise an expression inhibitor as described above and a pharmaceutically acceptable carrier or diluent. The composition may further comprise a colloidal dispersion system.

Pharmaceutical compositions that include expression inhibitors may include, but are not limited to, solutions, emulsions, and liposome-containing formulations. These compositions may be generated from a variety of components that include, but are not limited to, preformed liquids, self-emulsifying solids and self-emulsifying semisolids. The preparation of such compositions typically involves combining the expression inhibitor with one or more of the following: buffers, antioxidants, low molecular weight polypeptides, proteins, amino acids, carbohydrates including glucose, sucrose or dextrins, chelating agents such as EDTA, glutathione and other stabilizers and excipients. Neutral buffered saline or saline mixed with non-specific serum albumin are examples of suitable diluents.

In some embodiments, the compositions may be prepared and formulated as emulsions which are typically heterogeneous systems of one liquid dispersed in another in the form of droplets (see Idson in Pharmaceutical Dosage Forms, Vol. 1, Rieger and Banker (eds.), Marcek Dekker, Inc., N.Y., 1988.). Examples of naturally occurring emulsifiers used in emulsion formulations include acacia, beeswax, lanolin, lecithin and phosphatides.

In one embodiment, compositions including nucleic acids can be formulated as microemulsions. A microemulsion, as used herein refers to a system of water, oil and amphiphile, which is a single optically isotropic and thermodynamically stable liquid solution (see Rosoff in Pharmaceutical Dosage Forms, Vol. 1). The method of the invention may also use liposomes for the transfer and delivery of antisense oligonucleotides to the desired site.

Pharmaceutical compositions and formulations of expression inhibitors for topical administration may include transdermal patches, ointments, lotions, creams, gels, drops, suppositories, sprays, liquids and powders. Conventional pharmaceutical carriers, as well as aqueous, powder or oily bases and thickeners and the like may be used.

Modes of Administration

The pharmaceutical compositions comprising MAp19 inhibitory agents may be administered in a number of ways depending on whether a local or systemic mode of administration is most appropriate for the condition being treated. Additionally, as described herein above with respect to extracorporeal reperfusion procedures, MAp19 inhibitory agents can be administered via introduction of the compositions of the present invention to recirculating blood or plasma. Further, the compositions of the present invention can be delivered by coating or incorporating the compositions on or into an implantable medical device.

Systemic Delivery

As used herein, the terms “systemic delivery” and “systemic administration” are intended to include but are not limited to oral and parenteral routes including intramuscular (IM), subcutaneous, intravenous (IV), intra-arterial, inhalational, sublingual, buccal, topical, transdermal, nasal, rectal, vaginal and other routes of administration that effectively result in dispersement of the delivered agent to a single or multiple sites of intended therapeutic action. Preferred routes of systemic delivery for the present compositions include intravenous, intramuscular, subcutaneous and inhalational. It will be appreciated that the exact systemic administration route for selected agents utilized in particular compositions of the present invention will be determined in part to account for the agent's susceptibility to metabolic transformation pathways associated with a given route of administration. For example, peptidergic agents may be most suitably administered by routes other than oral.

MAp19 inhibitory antibodies and polypeptides can be delivered into a subject in need thereof by any suitable means. Methods of delivery of MAp19 antibodies and polypeptides include administration by oral, pulmonary, parenteral (e.g., intramuscular, intraperitoneal, intravenous (IV) or subcutaneous injection), inhalation (such as via a fine powder formulation), transdermal, nasal, vaginal, rectal, or sublingual routes of administration, and can be formulated in dosage forms appropriate for each route of administration.

By way of representative example, MAp19 inhibitory antibodies and peptides can be introduced into a living body by application to a bodily membrane capable of absorbing the polypeptides, for example the nasal, gastrointestinal and rectal membranes. The polypeptides are typically applied to the absorptive membrane in conjunction with a permeation enhancer. (See, e.g., Lee, V. H. L., Crit. Rev. Ther. Drug Carrier Syst. 5:69, 1988; Lee, V. H. L., J. Controlled Release 13:213, 1990; Lee, V. H. L. (ed.), Peptide and Protein Drug Delivery, Marcel Dekker, New York, 1991; DeBoer, A. G., et al., J. Controlled Release, 13:241, 1990.) For example, STDHF is a synthetic derivative of fusidic acid, a steroidal surfactant that is similar in structure to the bile salts, and has been used as a permeation enhancer for nasal delivery. (Lee, W. A., Biopharm. 22, November/December 1990.)

The MAp19 inhibitory antibodies and polypeptides may be introduced in association with another molecule, such as a lipid, to protect the polypeptides from enzymatic degradation. For example, the covalent attachment of polymers, especially polyethylene glycol (PEG), has been used to protect certain proteins from enzymatic hydrolysis in the body and thus prolong half-life (Fuertges, F., et al., J. Controlled Release 11:139, 1990). Many polymer systems have been reported for protein delivery (Bae, Y. H., et al., J. Controlled Release 9:271, 1989; Hori, R., et al., Pharm. Res. 6:813, 1989; Yamakawa, I., et al., J. Pharm. Sci. 79:505, 1990; Yoshihiro, I., et al., J. Controlled Release 10:195, 1989; Asano, M., et al., J. Controlled Release 9:111, 1989; Rosenblatt, J., et al., J. Controlled Release 9:195, 1989; Makino, K., J. Controlled Release 12:235, 1990; Takakura, Y., et al., J. Pharm. Sci. 78:117, 1989; Takakura, Y., et al., J. Pharm. Sci. 78:219, 1989).

Recently, liposomes have been developed with improved serum stability and circulation half-times (see, e.g., U.S. Pat. No. 5,741,516 to Webb). Furthermore, various methods of liposome and liposome-like preparations as potential drug carriers have been reviewed (see, e.g., U.S. Pat. No. 5,567,434 to Szoka; U.S. Pat. No. 5,552,157 to Yagi; U.S. Pat. No. 5,565,213 to Nakamori; U.S. Pat. No. 5,738,868 to Shinkarenko and U.S. Pat. No. 5,795,587 to Gao).

For transdermal applications, the MAp19 inhibitory antibodies and polypeptides may be combined with other suitable ingredients, such as carriers and/or adjuvants. There are no limitations on the nature of such other ingredients, except that they must be pharmaceutically acceptable for their intended administration, and cannot degrade the activity of the active ingredients of the composition. Examples of suitable vehicles include ointments, creams, gels, or suspensions, with or without purified collagen. The MAp19 inhibitory antibodies and polypeptides may also be impregnated into transdermal patches, plasters, and bandages, preferably in liquid or semi-liquid form.

The compositions of the present invention may be systemically administered on a periodic basis at intervals determined to maintain a desired level of therapeutic effect. For example, compositions may be administered, such as by subcutaneous injection, every two to four weeks or at least frequent intervals. The dosage regimen will be determined by the physician considering various factors that may influence the action of the combination of agents. These factors will include the extent of progress of the condition being treated, the patient's age, sex and weight, and other clinical factors. The dosage for each individual agent will vary as a function of the MAp19 inhibitory agent that is included in the composition, as well as the presence and nature of any drug delivery vehicle (e.g., a sustained release delivery vehicle). In addition, the dosage quantity may be adjusted to account for variation in the frequency of administration and the pharmacokinetic behavior of the delivered agent(s).

Local Delivery

As used herein, the term “local” encompasses application of a drug in or around a site of intended localized action, and may include for example topical delivery to the skin or other affected tissues, ophthalmic delivery, intrathecal (IT), intracerebroventricular (ICV), intra-articular, intracavity, intracranial or intravesicular administration, placement or irrigation. Local administration may be preferred to enable administration of a lower dose, to avoid systemic side effects, and for more accurate control of the timing of delivery and concentration of the active agents at the site of local delivery. Local administration provides a known concentration at the target site, regardless of interpatient variability in metabolism, blood flow, etc. Improved dosage control is provided by the direct mode of delivery.

Local delivery of a MAp19 inhibitory agent may be achieved in the context of surgical methods for treating a disease or condition, such as for example during procedures such as arterial bypass surgery, atherectomy, laser procedures, ultrasonic procedures, balloon angioplasty and stent placement. For example, a MAp19 inhibitor can be administered to a subject in conjunction with a balloon angioplasty procedure. A balloon angioplasty procedure involves inserting a catheter having a deflated balloon into an artery. The deflated balloon is positioned in proximity to the atherosclerotic plaque and is inflated such that the plaque is compressed against the vascular wall. As a result, the balloon surface is in contact with the layer of vascular endothelial cells on the surface of the blood vessel. The MAp19 inhibitory agent may be attached to the balloon angioplasty catheter in a manner that permits release of the agent at the site of the atherosclerotic plaque. The agent may be attached to the balloon catheter in accordance with standard procedures known in the art. For example, the agent may be stored in a compartment of the balloon catheter until the balloon is inflated, at which point it is released into the local environment. Alternatively, the agent may be impregnated on the balloon surface, such that it contacts the cells of the arterial wall as the balloon is inflated. The agent may also be delivered in a perforated balloon catheter such as those disclosed in Flugelman, et al., Circulation 85:1110-1117, 1992. See also published PCT Application WO 95/23161 for an exemplary procedure for attaching a therapeutic protein to a balloon angioplasty catheter. Likewise, the MAp19 inhibitory agent may be included in a gel or polymeric coating applied to a stent, or may be incorporated into the material of the stent, such that the stent elutes the MAp19 inhibitory agent after vascular placement.

MAp19 inhibitory compositions used in the treatment of arthritides and other musculoskeletal disorders may be locally delivered by intra-articular injection. Such compositions may suitably include a sustained release delivery vehicle. As a further example of instances in which local delivery may be desired, MAp19 inhibitory compositions used in the treatment of urogenital conditions may be suitably instilled intravesically or within another urogenital structure.

Coatings on a Medical Device

MAp19 inhibitory agents such as antibodies and inhibitory peptides may be immobilized onto (or within) a surface of an implantable or attachable medical device. The modified surface will typically be in contact with living tissue after implantation into an animal body. By “implantable or attachable medical device” is intended any device that is implanted into, or attached to, tissue of an animal body, during the normal operation of the device (e.g., stents and implantable drug delivery devices). Such implantable or attachable medical devices can be made from, for example, nitrocellulose, diazocellulose, glass, polystyrene, polyvinylchloride, polypropylene, polyethylene, dextran, Sepharose, agar, starch, nylon, stainless steel, titanium and biodegradable and/or biocompatible polymers. Linkage of the protein to a device can be accomplished by any technique that does not destroy the biological activity of the linked protein, for example by attaching one or both ends of the protein to the device. Attachment may also be made at one or more internal sites in the protein. Multiple attachments (both internal and at the ends of the protein) may also be used. A surface of an implantable or attachable medical device can be modified to include functional groups (e.g., carboxyl, amide, amino, ether, hydroxyl, cyano, nitrido, sulfanamido, acetylinic, epoxide, silanic, anhydric, succinimic, azido) for protein immobilization thereto. Coupling chemistries include, but are not limited to, the formation of esters, ethers, amides, azido and sulfanamido derivatives, cyanate and other linkages to the functional groups available on MAp19 antibodies or inhibitory peptides. MAp19 antibodies or inhibitory fragments can also be attached non-covalently by the addition of an affinity tag sequence to the protein, such as GST (Smith, D. B., et al., Gene 67:31, 1988), polyhistidines (Hochuli, E., et al., J. Chromatog. 411:77, 1987), or biotin. Such affinity tags may be used for the reversible attachment of the protein to a device.

Proteins can also be covalently attached to the surface of a device body, for example by covalent activation of the surface of the medical device. By way of representative example, matricellular protein(s) can be attached to the device body by any of the following pairs of reactive groups (one member of the pair being present on the surface of the device body, and the other member of the pair being present on the matricellular protein(s): hydroxyl/carboxylic acid to yield an ester linkage; hydroxyl/anhydride to yield an ester linkage; hydroxyl/isocyanate to yield a urethane linkage. A surface of a device body that does not possess useful reactive groups can be treated with radio-frequency discharge plasma (RFGD) etching to generate reactive groups in order to allow deposition of matricellular protein(s) (e.g., treatment with oxygen plasma to introduce oxygen-containing groups; treatment with propyl amino plasma to introduce amine groups).

MAp19 inhibitory agents comprising nucleic acid molecules such as antisense, RNAi- or DNA-encoding peptide inhibitors can be embedded in porous matrices attached to a device body. Representative porous matrices useful for making the surface layer are those prepared from tendon or dermal collagen, as may be obtained from a variety of commercial sources (e.g., Sigma and Collagen Corporation), or collagen matrices prepared as described in U.S. Pat. No. 4,394,370 to Jefferies and U.S. Pat. No. 4,975,527 to Koezuka. One collagenous material is termed UltraFiber™, and is obtainable from Norian Corp. (Mountain View, Calif.).

Certain polymeric matrices may also be employed if desired, and include acrylic ester polymers and lactic acid polymers, as disclosed, for example, in U.S. Pat. No. 4,526,909 to Urist and U.S. Pat. No. 4,563,489 to Urist. Particular examples of useful polymers are those of orthoesters, anhydrides, propylene-cofumarates, or a polymer of one or more α-hydroxy carboxylic acid monomers, (e.g., α-hydroxy acetic acid (glycolic acid) and/or α-hydroxy propionic acid (lactic acid)).

Treatment Regimens

In prophylactic applications, the pharmaceutical compositions are administered to a subject susceptible to, or otherwise at risk of, a condition associated with lectin-dependent complement activation in an amount sufficient to eliminate or reduce the risk of developing symptoms of the condition. In therapeutic applications, the pharmaceutical compositions are administered to a subject suspected of, or already suffering from, a condition associated with lectin-dependent complement activation in a therapeutically effective amount sufficient to relieve, or at least partially reduce, the symptoms of the condition. In both prophylactic and therapeutic regimens, compositions comprising MAp19 inhibitory agents may be administered in several dosages until a sufficient therapeutic outcome has been achieved in the subject. Application of the MAp19 inhibitory compositions of the present invention may be carried out by a single administration of the composition, or a limited sequence of administrations, for treatment of an acute condition, e.g., reperfusion injury or other traumatic injury. Alternatively, the composition may be administered at periodic intervals over an extended period of time for treatment of chronic conditions, e.g., arthritides or psoriasis.

The methods and compositions of the present invention may be used to inhibit inflammation and related processes that typically result from diagnostic and therapeutic medical and surgical procedures. To inhibit such processes, the MAp19 inhibitory composition of the present invention may be applied periprocedurally. As used herein “periprocedurally” refers to administration of the inhibitory composition preprocedurally and/or intraprocedurally and/or postprocedurally, i.e., before the procedure, before and during the procedure, before and after the procedure, before, during and after the procedure, during the procedure, during and after the procedure, or after the procedure. Periprocedural application may be carried out by local administration of the composition to the surgical or procedural site, such as by injection or continuous or intermittent irrigation of the site, or by systemic administration. Suitable methods for local perioperative delivery of MAp19 inhibitory agent solutions are disclosed in U.S. Pat. No. 6,420,432 to Demopulos and U.S. Pat. No. 6,645,168 to Demopulos. Suitable methods for local delivery of chondroprotective compositions including MAp19 inhibitory agent(s) are disclosed in International PCT Patent Application WO 01/07067 A2. Suitable methods and compositions for targeted systemic delivery of chondroprotective compositions including MAp19 inhibitory agent(s) are disclosed in International PCT Patent Application WO 03/063799 A2.

### IX. EXAMPLES

### Example 1

This example describes the generation of a mouse strain deficient in MAp19 (MAp19−/−) that is designed to express MASP-2.

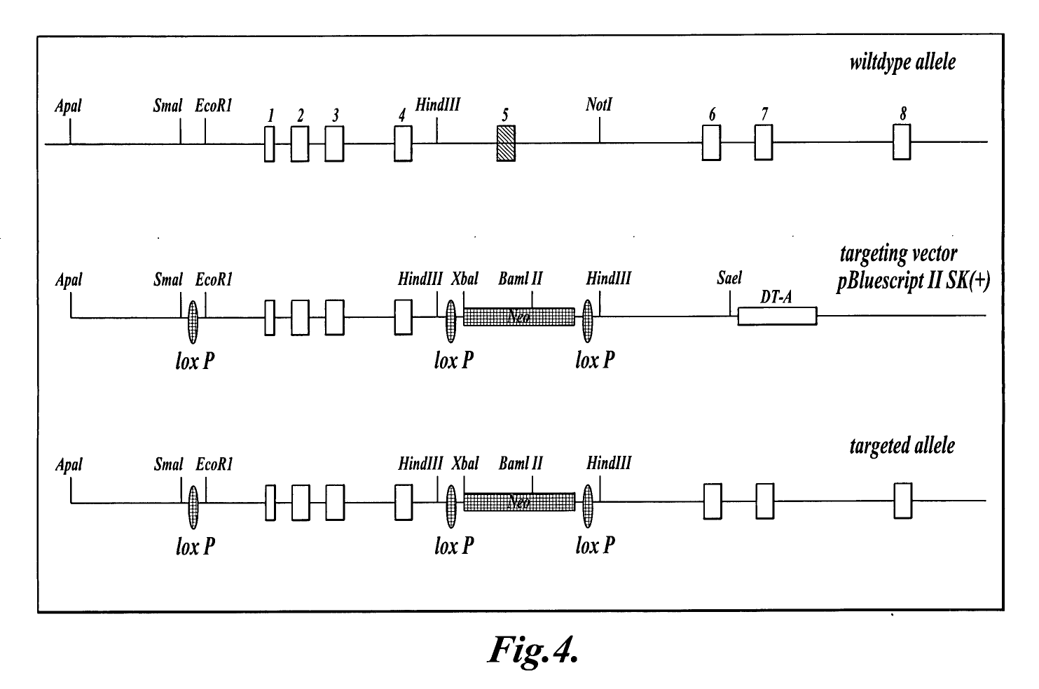

Materials and Methods: In order to generate a murine strain deficient in MAp19 but sufficient of MASP-2, a targeting vector was designed which included exons 1-4 of the BALB/c mouse MASP 2 gene and a neomycin resistance gene cassette instead of exon 5, a specific exon for MAp19 which encodes 4 amino acid residues at the C-terminal end as shown in FIG. 4. A diphtheria toxin A fragment gene (DTA) cassette was inserted into the 3′ flanking end of the vector and three lox p sites were inserted to perform conditional targeting. The targeting vector was electroporated into 129/Sv ES cells and transformants were selected for neomycin resistance. After selection of neomycin resistance, the Cre/lox system was used to excise the neomycin marker gene. A Cre construct was introduced into the transformed cells to achieve transient expression of Cre (as further described in Zou et al., Current Biology 4: 1099-2003 (1994). The resulting strain lacks exon 5 of the MASP 2 gene (and therefore is MAp19 deficient) but retains the remaining exons encoding MASP-2 (and therefore is expected to be MASP-2 sufficient). Positive ES clones were microinjected into C57BL/6 blastocytes and implanted into the uteri of foster mothers. Chimeric mice were mated with C57BL/6 female mice, and heterozygous (+/−) mice were backcrossed with C57BL/6 mice. Heterozygous (+/−) mice were intercrossed in order to obtain homozygous (−/−) mice in the C57BL/6 background.

Results and Phenotype: The resulting MAp19−/− mice developed normally and there was no significant difference in body weight between MAp19−/− and normal mice. In Northern blot analysis using poly(A)+ mRNA derived from MAp19−/− liver, the signal for MAp19 was not detected, whereas the MASP-2 mRNA was detected at a reduced level. In the serum of MAp19−/− mice, MAp19 was not detected by Western blot analysis. MASP-2 protein expression was reduced in the MAp19 deficient mice, as determined by Western blot analysis (data not shown). After incubation of MAp19−/− mouse serum with mannan-agarose, MAp19 and MASP-2 were not contained in the MBL-MASP complex coprecipitated with the agarose, although MASP-1 was coprecipitated. The plasma from homozygous MAp19−/− mice is deficient of lectin-pathway-mediated complement activation as further described in Example 2.

Generation of a MAp19−/− strain on a pure C57BL6 Background: The MAp19−/− mice are backcrossed with a pure C57BL6 line for nine generations prior to use of the MAp19−/− strain as an experimental animal model.

### Example 2

This example demonstrates that MAp19 may have a biological role in complement activation via the lectin pathway.

Methods and Materials:

Lectin pathway specific C4 cleavage assay: A C4 cleavage assay has been described by Petersen et al., J. Immunol. Methods 257:107, 2001, that measures lectin pathway activation resulting from lipoteichoic acid (LTA) from S. aureus which binds L-ficolin. The assay described in Example 14 was adapted to measure lectin pathway activation via MBL by coating the plate with LPS and mannan or zymosan prior to adding serum from MAp19−/− mice as described below. The assay was also modified to remove the possibility of C4 cleavage due to the classical pathway. This was achieved by using a sample dilution buffer containing 1 M NaCl, which permits high affinity binding of lectin pathway recognition components to their ligands, but prevents activation of endogenous C4, thereby excluding the participation of the classical pathway by dissociating the C1 complex. Briefly described, in the modified assay serum samples (diluted in high salt (1 M NaCl) buffer) are added to ligand-coated plates, followed by the addition of a constant amount of purified C4 in a buffer with a physiological concentration of salt. Bound recognition complexes containing MASP-2 cleave the C4, resulting in C4b deposition.

Assay Methods:

1) Nunc Maxisorb microtiter plates (Maxisorb, Nunc, cat. No. 442404, Fisher Scientific) were coated with 1 μg/ml mannan (M7504 Sigma) or any other ligand (e.g., such as those listed below) diluted in coating buffer (15 mM Na₂CO₃, 35 mM NaHCO₃, pH 9.6).

The following reagents were used in the assay:
a. Mannan (1 μg/well mannan (M7504 Sigma) in 100 μl coating buffer):
b. Zymosan (1 μg/well zymosan (Sigma) in 100 μl coating buffer);
c. LTA (1 μg/well in 100 ul coating buffer or 2 μg/well in 20 μl methanol)
d. 1 μg of the H-ficolin specific Mab 4H5 in coating buffer
e. PSA from Aerococcus viridans (2 μg/well in 100 μl coating buffer)
f. 100 μl/well of formalin-fixed S. aureus DSM20233 (OD₅₅₀=0.5) in coating buffer.

2) The plates were incubated overnight at 4° C.

3) After overnight incubation, the residual protein binding sites were saturated by incubated the plates with 0.1% HSA-TBS blocking buffer (0.1% (w/v) HSA in 10 mM Tris-CL, 140 mM NaCl, 1.5 mM NaN₃, pH 7.4) for 1-3 hours, then washing the plates 3× with TBS/tween/Ca²⁺ (TBS with 0.05% Tween 20 and 5 mM CaCl₂, 1 mM MgCl₂, pH 7.4).

4) Serum samples to be tested were diluted in MBL-binding buffer (1 M NaCl) and the diluted samples were added to the plates and incubated overnight at 4° C. Wells receiving buffer only were used as negative controls.

5) Following incubation overnight at 4° C., the plates were washed 3× with TBS/tween/Ca²⁺. Human C4 (100 μl/well of 1 μg/ml diluted in BBS (4 mM barbital, 145 mM NaCl, 2 mM CaCl₂, 1 mM MgCl₂, pH 7.4)) was then added to the plates and incubated for 90 minutes at 37° C. The plates were washed again 3× with TBS/tween/Ca²⁺.

6) C4b deposition was detected with an alkaline phosphatase-conjugated chicken anti-human C4c (diluted 1:1000 in TBS/tween/Ca²⁺), which was added to the plates and incubated for 90 minutes at room temperature. The plates were then washed again 3× with TBS/tween/Ca²⁺.

7) Alkaline phosphatase was detected by adding 100 μl of p-nitrophenyl phosphate substrate solution, incubating at room temperature for 20 minutes, and reading the OD₄₀₅ in a microtiter plate reader.

Results: As shown in FIG. 7, plasma from MAp19−/− mice is totally deficient in lectin-pathway-mediated complement activation on Mannan. This result is surprising and suggests that MAp19 may be a regulator of complement activation via the lectin-dependent pathway.

### Example 3

This example describes the generation of a mouse strain deficient in MASP-2 (MASP-2−/−) but sufficient of MAp19 (MAp19+/+).

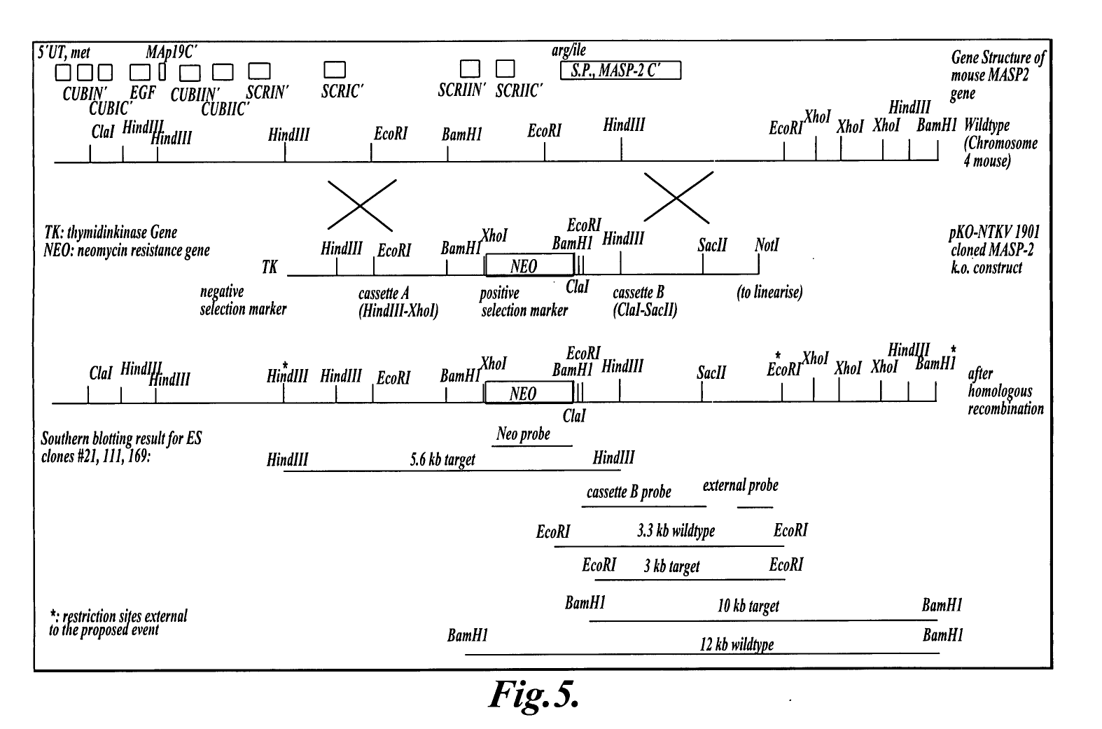

Materials and Methods: The targeting vector pKO-NTKV 1901 was designed to disrupt the three exons coding for the C-terminal end of murine MASP-2, including the exon that encodes the serine protease domain, as shown in FIG. 5. PKO-NTKV 1901 was used to transfect the murine ES cell line E14.1a (SV129 O1a). Neomycin resistant and Thymidine Kinase sensitive clones were selected. 600 ES clones were screened and of these, four different clones were identified and verified by southern blot to contain the expected selective targeting and recombination event as shown in FIG. 5. Chimeras were generated from these four positive clones by embryo transfer. The chimeras were then backcrossed in the genetic background C57/BL6 to create transgenic males. The transgenic males were crossed with females to generate F1s with 50% of the offspring showing heterozygosity for the disrupted MASP 2 gene. The heterozygous mice were intercrossed to generate homozygous MASP-2 deficient offspring, resulting in heterozygous and wild-type mice in the ration of 1:2:1, respectively.

Results and Phenotype: The resulting homozygous MASP-2−/− deficient mice were found to be viable and fertile and were verified to be MASP-2 deficient by southern blot to confirm the correct targeting event, by Northern blot to confirm the absence of MASP-2 mRNA, and by Western blot to confirm the absence of MASP-2 protein (data not shown). The presence of MAp19 mRNA and the absence of MASP-2 mRNA was further confirmed using time-resolved RT-PCR on a LightCycler machine. The MASP-2−/− mice do continue to express MAp19, MASP-1 and MASP-3 mRNA and protein as expected (data not shown). The presence and abundance of mRNA in the MASP-2−/− mice for Properidin, Factor B, Factor D, C4, C2 and C3 was assessed by LightCycler analysis and found to be identical to that of the wild-type littermate controls (data not shown). The plasma from homozygous MASP-2−/− mice is totally deficient of lectin-pathway-mediated complement activation and alternative pathway complement activation as further described in Example 4.

### Example 4

This example demonstrates that MASP-2 is required for complement activation via the alternative and the lectin pathway.

Methods and Materials:

Lectin pathway specific C4 cleavage assay: This assay was preformed as described in Example 2.

Results: As shown in FIGS. 8A-C, plasma from MASP-2−/− mice is totally deficient in lectin-pathway-mediated C4 complement activation on mannan (FIG. 8A) and on zymosan (FIG. 8B) coated plates in serum dilutions from MASP-2+/+ (crosses), MASP-2+/− (closed circles) and MASP-2−/− (closed triangles). FIG. 8C shows the relative C4 convertase activity on plates coated with zymosan (white bars) or mannan (shaded bars) from MASP-2−/+ mice (n=5) and MASP-2−/− mice (n=4) relative to wild-type mice (n=5) based on measuring the amount of C4b deposition normalized to wild-type serum. The error bars represent the standard deviation. These results clearly demonstrates that MASP-2, but not MASP-1 or MASP-3, is the effector component of the lectin pathway.

C3b Deposition Assay:
1) Nunc Maxisorb microtiter plates (Maxisorb, Nunc, cat. No. 442404, Fisher Scientific) are coated with 1 μg/well mannan (M7504 Sigma) or any other ligand diluted in coating buffer (15 mM Na₂CO₃, 35 mM NaHCO₃, pH 9.6) and incubated overnight at 4° C.

2) Residual protein binding sites are saturated by incubating the plate with 0.1% HSA-TBS blocking buffer (0.1% (w/v) HSA in 10 mM Tris-CL, 140 mM NaCl, 1.5 mM NaN₃, pH 7.4) for 1-3 hours.

3) Plates are washed in TBS/tw/Ca⁺⁺ (TBS with 0.05% Tween 20 and 5 mM CaCl₂) and diluted BBS is added to serum samples (4 mM barbital, 145 mM NaCl, 2 mM 1 mM MgCl₂, pH 7.4). Wells receiving only buffer are used as negative controls. A control set of serum samples obtained from wild-type or MASP-2−/− mice are C1q depleted prior to use in the assay. C1q-depleted mouse serum was prepared using protein-A-coupled Dynabeads (Dynal Biotech, Oslo, Norway) coated with rabbit anti-human C1q IgG (Dako, Glostrup, Denmark), according to the supplier's instructions.

4) Following incubation overnight at 4° C., and another wash with TBS/tw/Ca⁺⁺, converted and bound C3 is detected with a polyclonal anti-human-C3c Antibody (Dako A 062) diluted in TBS/tw/Ca⁺⁺ at 1:1000). The secondary antibody is goat anti-rabbit IgG (whole molecule) conjugated to alkaline-phosphatase (Sigma Immunochemicals A-3812) diluted 1:10,000 in TBS/tw/Ca⁺⁺. The presence of alternative complement pathway (AP) is determined by addition of 100 μl substrate solution (Sigma Fast p-Nitrophenyl Phosphate tablet sets, Sigma) and incubation at room temperature. Hydrolysis is monitored quantitatively by measuring the absorption at 405 nm in a microtiter plate reader. A standard curve is prepared for each analysis using serial dilutions of Plasma/serum samples.

Results: As shown in FIG. 9A-B, serum from MASP-2−/− mice tested in the C3b deposition assay results in very low levels of C3 activation on mannan (FIG. 9A) and on zymosan (FIG. 9B) coated plates. This result clearly demonstrates that MASP-2 is required to contribute the initial C3b to initiate the alternative pathway. This is a surprising result in view of the widely accepted view that complement factors C3, factor B, factor D and properdin form an independent functional alternative pathway in which C3 can undergo a spontaneous conformational change to a “C3b-like” form which then generates a fluid phase convertase iC3Bb and deposits C3b molecules on activation surfaces such as zymosan.

Recombinant MASP-2 Reconstitutes Lectin Pathway-Dependent C4 Activation in Serum from the MASP-2−/− Mice

In order to establish that the absence of MASP-2 was the direct cause of the loss of lectin pathway-dependent C4 activation in the MASP-2−/− mice, the effect of adding recombinant MASP-2 protein to serum samples was examined in the C4 cleavage assay described above. Functionally active murine MASP-2 and catalytically inactive murine MASP-2A (in which the active-site serine residue in the serine protease domain was substituted for the alanine residue) recombinant proteins were produced and purified. Pooled serum from 4 MASP-2−/− mice was pre-incubated with increasing protein concentrations of recombinant murine MASP-2 or inactive recombinant murine MASP-2A and C4 convertase activity was assayed as described above.

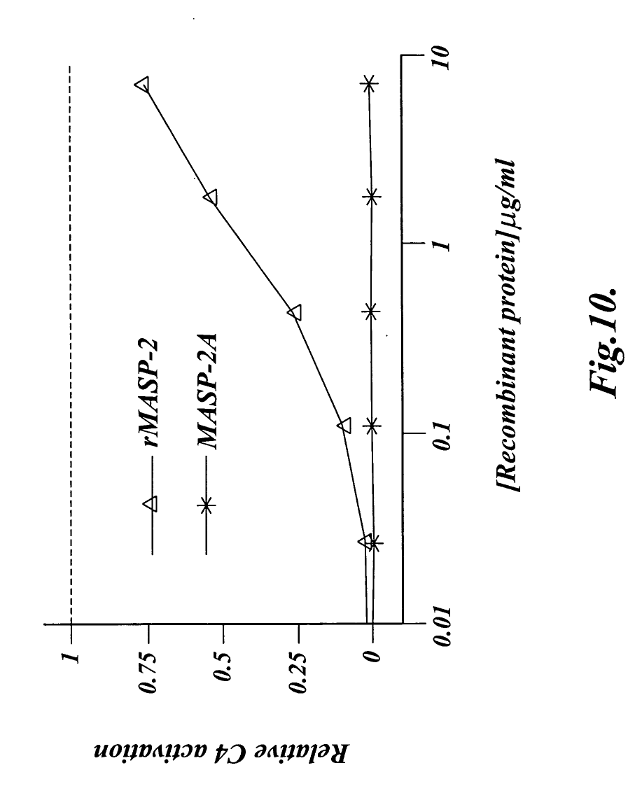

Results: As shown in FIG. 10, the addition of functionally active murine recombinant MASP-2 protein (shown as open triangles) to serum obtained from the MASP-2−/− mice restored lectin pathway-dependent C4 activation in a protein concentration dependent manner, whereas the catalytically inactive murine MASP-2A protein (shown as stars) did not restore C4 activation. The results shown in FIG. 10 are normalized to the C4 activation observed with pooled wild-type mouse serum (shown as a dotted line).

### Example 5

This example describes the generation of a transgenic mouse strain that is murine MAp19−/− and that expresses a human MAp19 transgene (SEQ ID NO: 42) (a murine MAp19 knockout and a human MAp19 knock-in).

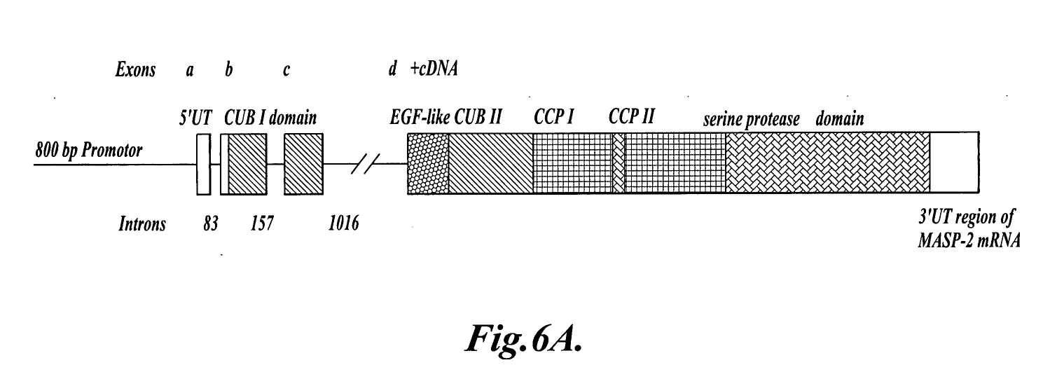

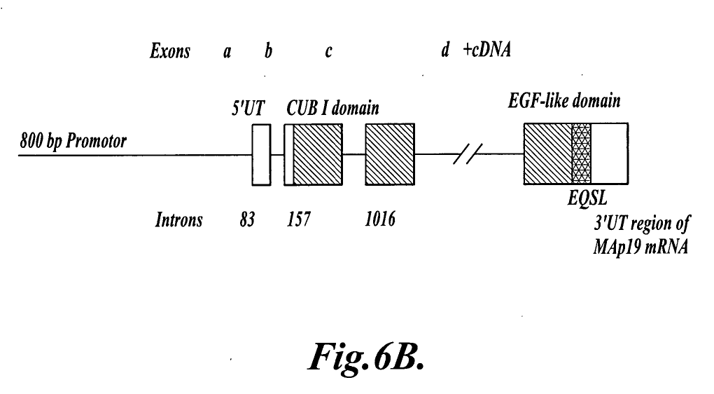

Materials and Methods: A minigene encoding human MAp19 called “mini hMAp19” (SEQ ID NO:42) as shown in FIG. 6B was constructed which includes the promoter region of the human MASP 2 gene, including the first 3 exons (exon 1 to exon 3) followed by the cDNA sequence that represents the coding sequence of the following 2 exons, thereby encoding the full-length MAp19 protein driven by its endogenous promoter. The mini hMAp19 construct was injected into fertilized eggs of MAp19−/− in order to replace the deficient murine MAp19 gene by transgenically expressed human MAp19.

### Example 6

This example describes the generation of a transgenic mouse strain that is murine MASP-2−/−, MAp19+/+ and that expresses a human MASP-2 transgene (SEQ ID NO: 41) (a murine MASP-2 knock-out and a human MASP-2 knock-in

Materials and Methods: A minigene encoding human MASP-2 called “mini hMASP-2” (SEQ ID NO:41) as shown in FIG. 6A was constructed which includes the promoter region of the human MASP 2 gene, including the first 3 exons (exon 1 to exon 3) followed by the cDNA sequence the represents the coding sequence of the following 8 exons, thereby encoding the full-length MASP-2 protein driven by its endogenous promoter. The mini hMASP-2 construct was injected into fertilized eggs of MASP-2−/− in order to replace the deficient murine MASP 2 gene by transgenically expressed human MASP-2.

### Example 7

This example describes the recombinant expression and protein production of recombinant full length human MAp19 and human MAp19 derived polypeptides.

Expression of Full-length human MAp19 in CHO Cells: The full length MAp19 protein can be expressed by transfecting the minigene construct (SEQ ID NO:42) shown in FIG. 6B (containing the promoter region of the human MASP 2 gene, including the first 3 exons (exon 1 to exon 3) followed by the cDNA sequence that represents the coding sequence of the full-length MAp19) into Chinese Hamster ovary cells (CHO). The human MAp19 protein is secreted into the culture media and purified as described below.

Expression of Full-length MAp19 in a baculovirus system: DNA fragments encoding the MASP-2 signal peptide followed either by aa 1-136 of MASP-2 (SEQ ID NO:4) (corresponding to the N-terminal CUB1 domain) or by aa 1-181 of MASP-2 (corresponding to the N-terminal CUB1 EGF domain), plus the EQSL residues corresponding to the MAp19 sequence were amplified by PCR using Vent_{R} polymerase and pBS-MASP-2 as a template, according to established procedures. The sequence of the sense primer (5′-CGGGATCCATGAGGCTGCTGACCCTC-3′ SEQ ID NO:28) introduced a BamHI restriction site (underlined) at the 5′ end of both PCR products.

The antisense primer for MAp19 (5′-GGGGTACCCTAGAGGCTCTGCTCTGAGCAGGTGCGCTT-3′ SEQ ID NO:29) introduced the EQSL amino acid sequence (boldface and underlined) followed by a stop codon (boldface) and a KpnI site (underlined) at the 3′ end of the PCR product. The amplified DNA fragments were digested with BamHI and KpnI, and BamHI and EcoRI for MAp19 and the CUB-EGF fragment, respectively, and cloned into the corresponding sites of the pFastBac1 vector. The resulting constructs were characterized by restriction mapping and checked by dsDNA sequencing (Genome Express, Grenoble, France).

Production of Recombinant human MAp19 Polypeptides

A method for producing recombinant MASP-2 derived polypeptides is described in Thielens, N. M., et al., J. Immunol. 166:5068-5077, 2001, which is modified to produce MAp19 polypeptides. Briefly, the Spodoptera frugiperda insect cells (Ready-Plaque Sf9 cells obtained from Novagen, Madison, Wis.) are grown and maintained in Sf900II serum-free medium (Life Technologies) supplemented with 50 IU/ml penicillin and 50 mg/ml streptomycin (Life Technologies). The Trichoplusia ni (High Five) insect cells (provided by Jadwiga Chroboczek, Institut de Biologie Structurale, Grenoble, France) are maintained in TC100 medium (Life Technologies) containing 10% FCS (Dominique Dutscher, Brumath, France) supplemented with 50 IU/ml penicillin and 50 mg/ml streptomycin. Recombinant baculoviruses are generated using the Bac-to-Bac system (Life Technologies). The bacmid DNA is purified using the Qiagen midiprep purification system (Qiagen) and is used to transfect Sf9 insect cells using cellfectin in Sf900 II SFM medium (Life Technologies) as described in the manufacturer's protocol. Recombinant virus particles are collected 4 days later, titrated by virus plaque assay, and amplified as described by King and Possee, in The Baculovirus Expression System: A Laboratory Guide, Chapman and Hall Ltd., London, pp. 111-114, 1992.

High Five cells (1.75×10⁷ cells/175-cm² tissue culture flask) are infected with the recombinant viruses containing MAp19 polypeptides at a multiplicity of infection of 2 in Sf900 II SFM medium at 28° C. for 96 h. The supernatants are collected by centrifugation and diisopropyl phosphorofluoridate is added to a final concentration of 1 mM.

The MAp19 polypeptides are secreted in the culture medium. A purification method for MAp19 protein has been described by Thielens, N. M., et al., J. Immunol. 166:5068-5077, 2001. Briefly, the MAp19 polypeptides are purified from cell culture supernatants by anion-exchange chromatography on a Q-Sepharose-Fast Flow column followed by gel filtration in the presence of Ca2+ ions on a TSK G3000 SWG column.

### Example 8

This example describes a method of producing polyclonal antibodies against MAp19 polypeptides.

Materials and Methods:

MAp19 Antigens: Polyclonal anti-human MAp19 antiserum is produced by immunizing rabbits with the following isolated MAp19 polypeptides: full length human MAp19 (SEQ ID NO:3) isolated as described in Example 7; and recombinant MAp19 polypeptides and synthesized peptides comprising the following: SEQ ID NO:8 (CUBI domain); SEQ ID NO:9 (CUBI peptide); SEQ ID NO:10 (CUBI peptide); SEQ ID NO:11 (CUBI peptide); SEQ ID NO:12 (peptide comprising SEQSL C-terminus) and SEQ ID NO:13 (MAp19 C-terminal peptide)

Polyclonal antibodies: Six week old rabbits, primed with BCG (bacillus Calmette-Guerin vaccine) are immunized by injecting 100 μg of MAp19 polypeptide at 100 μg/ml in sterile saline solution. Injections are done every 4 weeks, with antibody titer monitored by ELISA assay as described in Example 9. Culture supernatants are collected for antibody purification by protein A affinity chromatography.

### Example 9

This example describes a method for producing murine monoclonal antibodies against human MAp19 polypeptides.

Materials and Methods:

Male A/J mice (Harlan, Houston, Tex.), 8-12 weeks old, are injected subcutaneously with 100 μg human MAp19 polypeptide (made as described in Example 7) in complete Freund's adjuvant (Difco Laboratories, Detroit, Mich.) in 200 μl of phosphate buffered saline (PBS) pH7.4. At two-week intervals the mice are twice injected subcutaneously with 50 μg of human rMAp19 polypeptide in incomplete Freund's adjuvant. On the fourth week the mice are injected with 50 μg of human MAp19 polypeptide in PBS and are fused 4 days later.

For each fusion, single cell suspensions are prepared from the spleen of an immunized mouse and used for fusion with Sp2/0 myeloma cells. 5×10⁸ of the Sp2/0 and 5×10⁸ spleen cells are fused in a medium containing 50% polyethylene glycol (M.W. 1450) (Kodak, Rochester, N.Y.) and 5% dimethylsulfoxide (Sigma Chemical Co., St. Louis, Mo.). The cells are then adjusted to a concentration of 1.5×10⁵ spleen cells per 200 μl of the suspension in Iscove medium (Gibco, Grand Island, N.Y.), supplemented with 10% fetal bovine serum, 100 units/ml of penicillin, 100 μg/ml of streptomycin, 0.1 mM hypoxanthine, 0.4 μM aminopterin and 16 μM thymidine. Two hundred microliters of the cell suspension are added to each well of about twenty 96-well microculture plates. After about ten days culture supernatants are withdrawn for screening for reactivity with purified factor MAp19 in an ELISA assay.

ELISA Assay: Wells of Immulon 2 (Dynatech Laboratories, Chantilly, Va.) microtest plates are coated by adding 50 μl of purified hMAp19 at 50 ng/ml overnight at room temperature. The low concentration of MAp19 for coating enables the selection of high-affinity antibodies. After the coating solution is removed by flicking the plate, 200 μl of BLOTTO (non-fat dry milk) in PBS is added to each well for one hour to block the non-specific sites. An hour later, the wells are then washed with a buffer PBST (PBS containing 0.05% Tween 20). Fifty microliters of culture supernatants from each fusion well is collected and mixed with 50 μl of BLOTTO and then added to the individual wells of the microtest plates. After one hour of incubation, the wells are washed with PBST. The bound murine antibodies are then detected by reaction with horseradish peroxidase (HRP) conjugated goat anti-mouse IgG (Fc specific) (Jackson ImmunoResearch Laboratories, West Grove, Pa.) and diluted at 1:2,000 in BLOTTO. Peroxidase substrate solution containing 0.1% 3,3,5,5 tetramethyl benzidine (Sigma, St. Louis, Mo.) and 0.0003% hydrogen peroxide (Sigma) is added to the wells for color development for 30 minutes. The reaction is terminated by addition of 50 μl of 2M H₂SO₄ per well. The Optical Density at 450 nm of the reaction mixture is read with a BioTek ELISA Reader (BioTek Instruments, Winooski, Vt.).

Binding Assay for Cross-Reactivity to MASP-2 polypeptide: Culture supernatants that test positive in the MAp19 ELISA assay described above can be tested in a binding assay to determine if bind to MASP-2 using the following assay. A similar assay can also be used to determine if the MAp19 inhibitory agents bind to other antigens in the complement system.

Polystyrene microtiter plate wells (96-well medium binding plates, Corning Costar, Cambridge, Mass.) are coated with MASP-2 (20 ng/100 μl/well); Advanced Research Technology, San Diego, Calif.) in phosphate-buffered saline (PBS) pH 7.4 overnight at 4° C. After aspirating the MASP-2 solution, wells are blocked with PBS containing 1% bovine serum albumin (BSA; Sigma Chemical) for 2 h at room temperature. Wells without MASP-2 coating serve as the background controls. Aliquots of hybridoma supernatants or purified anti-MAp19 MoAbs, at varying concentrations in blocking solution, are added to the wells. Following a 2 h incubation at room temperature, the wells are extensively rinsed with PBS. MASP-2-bound anti-MAp19 MoAb is detected by the addition of peroxidase-conjugated goat anti-mouse IgG (Sigma Chemical) in blocking solution, which is allowed to incubate for 1 h at room temperature. The plate is rinsed again thoroughly with PBS, and 100 μl of 3,3′,5,5′-tetramethyl benzidine (TMB) substrate (Kirkegaard and Perry Laboratories, Gaithersburg, Md.) is added. The reaction of TMB is quenched by the addition of 100 μl of 1M phosphoric acid, and the plate is read at 450 nm in a microplate reader (SPECTRA MAX 250, Molecular Devices, Sunnyvale, Calif.).

Assay for Lectin Complement Inhibition: Once positive wells are identified that contain anti-MAp19 antibodies, the culture supernatants from these positive wells can be tested by the ability to inhibit complement activation in a functional assay such as the C4 cleavage assay as described in Example 2. The cells in positive wells are then cloned by limiting dilution. The MoAbs are tested again for reactivity with MAp19 in an ELISA assay as described above. The selected hybridomas are grown in spinner flasks and the spent culture supernatant collected for antibody purification by protein A affinity chromatography.

### Example 10

This example describes a method of producing human MAp19 antibodies that do not bind to human MASP-2.

Methods and Materials: The MAp19−/− murine strain (as described in Example 1) or a MAp19-MASP-2−/− murine strain expressing human MASP-2 obtained by crossing the MAp19−/− murine strain with the MASP-2−/− murine strain expressing the human MASP-2 minigene (obtained as described in Example 6) are injected with human MAp19 antigen as described in Example 9. Monoclonal murine anti-human MAp19 antibodies are produced using hybridomas as described in Example 9. Murine anti-human MAp19 antibodies that do not cross react with human MASP-2 are identified by screening using a binding assay described in Example 9 which can be confirmed by Western blot.

The novel methods of producing anti-MAp19 antibodies using the murine MAp19 knockout and/or the double MAp19, MASP-2 knockout and human MASP-2 knockin are useful because they provide the opportunity to identify specific MAp19 epitopes that are not present on MASP-2. This is due to the fact that the endogenous MASP-2 will be recognized as a self-antigen and non-immunogenic, whereas the MAp19 polypeptide will be recognized as a foreign antigen.

The anti-MAp19 antibodies identified that do not bind to MASP-2 can be screened in a functional assay, such as a C4 cleavage assay (as described in Example 2) for the ability to inhibit lectin-dependent complement activation. Anti-MAp19 antibodies that inhibit lectin-dependent complement activation can be humanized for therapeutic applications as described in Example 13.

### Example 11

This example describes the use of a MAp19−/− knockout mouse expressing human MAp19 for use as a model in which to screen for MAp19 inhibitory agents.

Materials and Methods: A MAp19−/− mouse which expresses MASP-2 (as described in Example 1) is transfected with a human MAp19 minigene construct (as described in Example 5), resulting in a murine strain that is murine MAp19−/−, human MAp19+ and murine MASP-2+. This murine strain is expected to have a functional lectin-dependent complement activation pathway due to the high level of homology between murine and human MAp19. This animal model can be used as for the identification and efficacy of MAp19 inhibitory agents such as human anti-MAp19 antibodies, MAp19 inhibitory peptides and nonpeptides, and compositions comprising MAp19 inhibitory agents by administering these agents in vivo, or by deriving a cell line testing in vitro. For example, the animal model is exposed to a compound or agent that is known to trigger lectin-dependent complement activation, and a MAp19 inhibitory agent is administered to the animal model at a sufficient time and concentration to elicit a reduction of disease symptoms in the exposed animal.

In addition, the murine MAp19−/−, human MAp19+, murine MASP-2+ mice may be used to generate cell lines containing one or more cell types involved in a disease associated with lectin-dependent complement activation which can be used as a cell culture model for that disorder. The generation of continuous cell lines from transgenic animals is well known in the art, for example see Small, J. A., et al., Mol. Cell Biol., 5:642-48, 1985.

### Example 12

This example describes a method of producing human antibodies against human MAp19 in a MAp19−/− knockout mouse that expresses human immunoglobulins.

Materials and Methods:

A MAp19−/− mouse was generated as described in Example 1. A homozygous MAp19−/− mouse is then each crossed with a mouse derived from an embryonic stem cell line engineered to contain targeted disruptions of the endogenous immunoglobulin heavy chain and light chain loci and expression of at least a segment of the human immunoglobulin locus. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination. The transgenic mammals resulting from this process are capable of functionally rearranging the immunoglobulin component sequences and expressing a repertoire of antibodies of various isotypes encoded by human immunoglobulin genes, without expressing endogenous immunoglobulin genes. The production and properties of mammals having these properties is described, for example in Thomson, A. D., Nature 148:1547-1553, 1994, and Sloane, B. F., Nature Biotechnology 14:826, 1996. Genetically engineered strains of mice in which the mouse antibody genes are inactivated and functionally replaced with human antibody genes is commercially available (e.g., XenoMouse®, available from Abgenix, Fremont, Calif.).

The resulting offspring mice are injected with MAp19 derived antigens as previously described and are capable of producing human MoAb against human MAp19 that are suitable for use in human therapy.

### Example 13

This example describes the generation and production of humanized murine anti MAp19 antibodies and antibody fragments.

A murine anti-MAp19 monoclonal antibody is generated in Male A/J mice as described in Example 9. The murine antibody is then humanized as described below to reduce its immunogenicity by replacing the murine constant regions with their human counterparts to generate a chimeric IgG and Fab fragment of the antibody, which is useful for inhibiting the adverse effects of MAp19-dependent complement activation in human subjects in accordance with the present invention.

1. Cloning of anti-MAp19 variable region genes from murine hybridoma cells. Total RNA is isolated from the hybridoma cells secreting anti-MAp19 MoAb (obtained as described in Example 9) using RNAzol following the manufacturer's protocol (Biotech, Houston, Tex.). First strand cDNA is synthesized from the total RNA using oligo dT as the primer. PCR is performed using the immunoglobulin constant C region-derived 3′ primers and degenerate primer sets derived from the leader peptide or the first framework region of murine V_{H} or V_{K} genes as the 5′ primers. Anchored PCR is carried out as described by Chen and Platsucas (Chen, P. F., Scand. J. Immunol. 35:539-549, 1992). For cloning the V_{K} gene, double-stranded cDNA is prepared using a NotI-MAK1 primer (5′-TGCGGCCGCTGTAGGTGCTGTCTTT-3′ SEQ ID NO:30). Annealed adaptors AD1 (5′-GGAATTCACTCGTTATTCTCGGA-3′ SEQ ID NO:31) and AD2 (5′-TCCGAGAATAACGAGTG-3′ SEQ ID NO:32) are ligated to both 5′ and 3′ termini of the double-stranded cDNA. Adaptors at the 3′ ends are removed by NotI digestion. The digested product is then used as the template in PCR with the AD1 oligonucleotide as the 5′ primer and MAK2 (5′-CATTGAAAGCTTTGGGGTAGAAGTTGTTC-3′ SEQ ID NO:33) as the 3′ primer. DNA fragments of approximately 500 bp are cloned into pUC19. Several clones are selected for sequence analysis to verify that the cloned sequence encompasses the expected murine immunoglobulin constant region. The NotI-MAK1 and MAK2 oligonucleotides are derived from the V_{K} region and are 182 and 84 bp, respectively, downstream from the first base pair of the C kappa gene. Clones are chosen that include the complete V_{K} and leader peptide.

For cloning the V_{H} gene, double-stranded cDNA is prepared using the NotI-MAG1 primer (5′-CGCGGCCGCAGCTGCTCAGAGTGTAGA-3′ SEQ ID NO:34). Annealed adaptors AD1 and AD2 are ligated to both 5′ and 3′ termini of the double-stranded cDNA. Adaptors at the 3′ ends are removed by NotI digestion. The digested product are used as the template in PCR with the AD1 oligonucleotide and MAG2 (5′-CGGTAAGCTTCACTGGCTCAGGGAAATA-3′ SEQ ID NO:35) as primers. DNA fragments of 500 to 600 bp in length are cloned into pUC19. The NotI-MAG1 and MAG2 oligonucleotides are derived from the murine Cγ.7.1 region, and are 180 and 93 bp, respectively, downstream from the first bp of the murine Cγ.7.1 gene. Clones are chosen that encompass the complete V_{H} and leader peptide.

2. Construction of Expression Vectors for Chimeric MAp19 IgG and Fab. The cloned V_{H} and V_{K} genes described above are used as templates in a PCR reaction to add the Kozak consensus sequence to the 5′ end and the splice donor to the 3′ end of the nucleotide sequence. After the sequences are analyzed to confirm the absence of PCR errors, the V_{H} and V_{K} genes are inserted into expression vector cassettes containing human Cγ1 and C kappa respectively, to give pSV2neoV_{H}-huCγ1 and pSV2neoV-huCγ. CsCl gradient-purified plasmid DNAs of the heavy- and light-chain vectors are used to transfect COS cells by electroporation. After 48 hours, the culture supernatant is tested by ELISA to confirm the presence of approximately 200 ng/ml of chimeric IgG. The cells are harvested and total RNA is prepared. First strand cDNA is synthesized from the total RNA using oligo dT as the primer. This cDNA is used as the template in PCR to generate the Fd and kappa DNA fragments. For the Fd gene, PCR is carried out using 5′-AAGAAGCTTGCCGCCACCATGGATTGGCTGTGGAACT-3′ (SEQ ID NO:36) as the 5′ primer and a CH1-derived 3′ primer (5′-CGGGATCCTCAAACTTTCTTGTCCACCTTGG-3′ SEQ ID NO:37). The DNA sequence is confirmed to contain the complete V_{H} and the CH1 domain of human IgG1. After digestion with the proper enzymes, the Fd DNA fragments are inserted at the HindIII and BamHI restriction sites of the expression vector cassette pSV2dhfr-TUS to give pSV2dhfrFd. The pSV2 plasmid is commercially available and consists of DNA segments from various sources: pBR322 DNA (thin line) contains the pBR322 origin of DNA replication (pBR ori) and the lactamase ampicillin resistance gene (Amp); SV40 DNA, represented by wider hatching and marked, contains the SV40 origin of DNA replication (SV40 ori), early promoter (5′ to the dhfr and neo genes), and polyadenylation signal (3′ to the dhfr and neo genes). The SV40-derived polyadenylation signal (pA) is also placed at the 3′ end of the Fd gene.

For the kappa gene, PCR is carried out using 5′-AAGAAAGCTTGCCGCCACCATGTTCTCACTAGCTCT-3′ (SEQ ID NO:38) as the 5′ primer and a C_{K}-derived 3′ primer (5′-CGGGATCCTTCTCCCTCTAACACTCT-3′ SEQ ID NO:39). DNA sequence is confirmed to contain the complete V_{K} and human C_{K} regions. After digestion with proper restriction enzymes, the kappa DNA fragments are inserted at the HindIII and BamHI restriction sites of the expression vector cassette pSV2neo-TUS to give pSV2neoK. The expression of both Fd and kappa genes are driven by the HCMV-derived enhancer and promoter elements. Since the Fd gene does not include the cysteine amino acid residue involved in the inter-chain disulfide bond, this recombinant chimeric Fab contains non-covalently linked heavy- and light-chains. This chimeric Fab is designated as cFab.

To obtain recombinant Fab with an inter-heavy and light chain disulfide bond, the above Fd gene may be extended to include the coding sequence for additional 9 amino acids (EPKSCDKTH, SEQ ID NO:40) from the hinge region of human IgG1. The BstEII-BamHI DNA segment encoding 30 amino acids at the 3′ end of the Fd gene may be replaced with DNA segments encoding the extended Fd, resulting in pSV2dhfrFd/9aa.

3. Expression and Purification of Chimeric Anti-MAp19 IgG

To generate cell lines secreting chimeric anti-MAp19 IgG, NSO cells are transfected with purified plasmid DNAs of pSV2neoV_{H}-huC.γ1 and pSV2neoV-huC kappa by electroporation. Transfected cells are selected in the presence of 0.7 mg/ml G418. Cells are grown in a 250 ml spinner flask using serum-containing medium.

Culture supernatant of 100 ml spinner culture is loaded on a 10-ml PROSEP-A column (Bioprocessing, Inc., Princeton, N.J.). The column is washed with 10 bed volumes of PBS. The bound antibody is eluted with 50 mM citrate buffer, pH 3.0. Equal volume of 1 M Hepes, pH 8.0 is added to the fraction containing the purified antibody to adjust the pH to 7.0. Residual salts are removed by buffer exchange with PBS by Millipore membrane ultrafiltration (M.W. cut-off: 3,000). The protein concentration of the purified antibody is determined by the BCA method (Pierce).

4. Expression and Purification of Chimeric Anti-MAp19 Fab

To generate cell lines secreting chimeric anti-MAp19-2 Fab, CHO cells are transfected with purified plasmid DNAs of pSV2dhfrFd (or pSV2dhfrFd/9aa) and pSV₂neokappa, by electroporation. Transfected cells are selected in the presence of G418 and methotrexate. Selected cell lines are amplified in increasing concentrations of methotrexate. Cells are single-cell subcloned by limiting dilution. High-producing single-cell subcloned cell lines are then grown in 100 ml spinner culture using serum-free medium.

Chimeric anti-MAp19 Fab is purified by affinity chromatography using a mouse anti-idiotypic MoAb to the MAp19 MoAb. An anti-idiotypic MAp19 MoAb can be made by immunizing mice with a murine anti-MAp19 MoAb conjugated with keyhole limpet hemocyanin (KLH) and screening for specific MoAb binding that can be competed with human MAp19. For purification, 100 ml of supernatant from spinner cultures of CHO cells producing cFab or cFab/9aa are loaded onto the affinity column coupled with an anti-idiotype MAp19 MoAb. The column is then washed thoroughly with PBS before the bound Fab is eluted with 50 mM diethylamine, pH 11.5. Residual salts are removed by buffer exchange as described above. The protein concentration of the purified Fab is determined by the BCA method (Pierce).

The ability of the chimeric MAp19 IgG, cFab, and cFAb/9aa to inhibit MAp19-dependent complement pathways may be determined by using the inhibitory assays described in Example 2 and Example 14.

### Example 14

This example describes an in vitro C4 cleavage assay used as a functional screen to identify MAp19 inhibitory agents capable of blocking lectin-dependent complement activation via L-ficolin/P35, H-ficolin, M-ficolin or mannan.

C4 Cleavage Assay: A C4 cleavage assay has been described by Petersen, S. V., et al., J. Immunol. Methods 257:107, 2001, which measures lectin pathway activation resulting from lipoteichoic acid (LTA) from S. aureus which binds L-ficolin.

Reagents: Formalin-fixed S. aureous (DSM20233) is prepared as follows: bacteria is grown overnight at 37° C. in tryptic soy blood medium, washed three times with PBS, then fixed for 1 h at room temperature in PBS/0.5% formalin, and washed a further three times with PBS, before being resuspended in coating buffer (15 mM Na₂CO₃, 35 mM NaHCO₃, pH 9.6).

Assay: The wells of a Nunc MaxiSorb microtiter plate (Nalgene Nunc International, Rochester, N.Y.) are coated with: 100 μl of formalin-fixed S. aureus DSM20233 (OD₅₅₀=0.5) in coating buffer with 1 μg of L-ficolin in coating buffer. After overnight incubation, wells are blocked with 0.1% human serum albumin (HSA) in TBS (10 mM Tris-HCl, 140 mM NaCl, pH 7.4), then are washed with TBS containing 0.05% Tween 20 and 5 mM CaCl₂ (wash buffer). Human serum samples are diluted in 20 mM Tris-HCl, 1 M NaCl, 10 mM CaCl₂, 0.05% Triton X-100, 0.1% HSA, pH 7.4, which prevents activation of endogenous C4 and dissociates the C1 complex (composed of C1q, C1r and C1s). MAp19 inhibitory agents, including anti-MAp19 MoAbs and inhibitory peptides are added to the serum samples in varying concentrations. The diluted samples are added to the plate and incubated overnight at 4° C. After 24 hours, the plates are washed thoroughly with wash buffer, then 0.1 μg of purified human C4 (obtained as described in Dodds, A. W., Methods Enzymol. 223:46, 1993) in 100 μl of 4 mM barbital, 145 mM NaCl, 2 mM CaCl₂, 1 mM MgCl₂, pH 7.4 is added to each well. After 1.5 h at 37° C., the plates are washed again and C4b deposition is detected using alkaline phosphatase-conjugated chicken anti-human C4c (obtained from Immunsystem, Uppsala, Sweden) and measured using the colorimetric substrate ρ-nitrophenyl phosphate.

C4 Assay on mannan: The assay described above is adapted to measure lectin pathway activation via MBL by coating the plate with LPS and mannan prior to adding serum mixed with various MAp19 inhibitory agents.

C4 assay on H-ficolin (Hakata Ag): The assay described above is adapted to measure lectin pathway activation via H-ficolin by coating the plate with LPS and H-ficolin prior to adding serum mixed with various MAp19 inhibitory agents.

### Example 15

The following assay demonstrates the presence of classical pathway activation in wild-type and MASP-2−/− mice.

Methods: Immune complexes were generated in situ by coating microtiter plates (Maxisorb, Nunc, cat. No. 442404, Fisher Scientific) with 0.1% human serum albumin in 10 mM Tris, 140 mM NaCl, pH 7.4 for 1 hour at room temperature, followed by overnight incubation at 4° C. with sheep anti-whole serum antiserum (Scottish Antibody Production Unit, Carluke, Scotland) diluted 1:1000 in TBS/tween/Ca²⁺. Serum samples were obtained from wild-type and MASP-2−/− mice and added to the coated plates. Control samples were prepared in which C1q was depleted from wild-type and MASP-2−/− serum samples. C1q-depleted mouse serum was prepared using protein-A-coupled Dynabeads (Dynal Biotech, Oslo, Norway) coated with rabbit anti-human C1q IgG (Dako, Glostrup, Denmark), according to the supplier's instructions. The plates were incubated for 90 minutes at 37° C. Bound C3b was detected with a polyclonal anti-human-C3c Antibody (Dako A 062) diluted in TBS/tw/Ca⁺⁺ at 1:1000. The secondary antibody was goat anti-rabbit IgG.

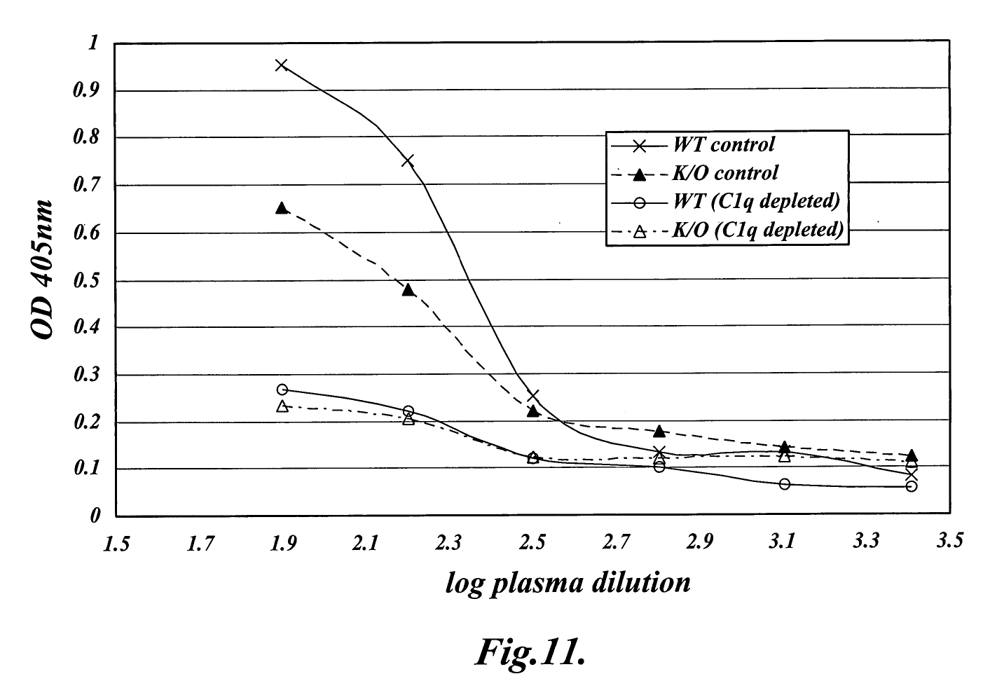

Results: FIG. 11 shows the relative C3b deposition levels on plates coated with IgG in wild-type serum, MASP-2−/− serum, C1q-depleted wild-type and C1q-depleted MASP-2−/− serum. These results demonstrate that the classical pathway is intact in the MASP-2−/− mouse strain.

### Example 16

The following assay is used to test whether a MAp19 inhibitory agent blocks the classical pathway by analyzing the effect of a MAp19 inhibitory agent under conditions in which the classical pathway is initiated by immune complexes.

Methods: To test the effect of a MAp19 inhibitory agent on conditions of complement activation where the classical pathway is initiated by immune complexes, triplicate 50 μl samples containing 90% NHS are incubated at 37° C. in the presence of 10 μg/ml immune complex (IC) or PBS, and parallel triplicate samples (+/−IC) are also included which contain 200 nM anti-properdin monoclonal antibody during the 37° C. incubation. After a two hour incubation at 37° C., 13 mM EDTA is added to all samples to stop further complement activation and the samples are immediately cooled to 5° C. The samples are then stored at −70° C. prior to being assayed for complement activation products (C3a and sC5b-9) using ELISA kits (Quidel, Catalog Nos. A015 and A009) following the manufacturer's instructions.

### Example 17

This example demonstrates that the lectin-dependent complement activation system is activated in the reperfusion phase following abdominal aortic aneurysm repair.

Experimental Rationale and Design: Patients undergoing abdominal aortic aneurysm (AAA) repair are subject to an ischemia-reperfusion injury which is largely mediated by complement activation. We investigated the role of the lectin pathway of complement activation in ischemia-reperfusion injury in patients undergoing AAA repair. The consumption of mannan-binding lectin (MBL) in serum was used to measure the amount of lectin pathway activation that occurred during reperfusion.

Patient Serum Sample Isolation: A total of 23 patients undergoing elective infrarenal AAA repair and 8 control patients undergoing major abdominal surgery were included in this study.

For the patients under going AAA repair, systemic blood samples were taken from each patient's radial artery (via an arterial line) at four defined time points during the procedure: time point 1: induction of anaesthesia; time point 2: just prior to aortic clamping; time point 3: just prior to aortic clamp removal; and time point 4: during reperfusion.

For the control patients undergoing major abdominal surgery, systemic blood samples were taken at induction of anaesthesia and at two hours after the start of the procedure.

Assay for levels of MBL: Each patient plasma sample was assayed for levels of mannan-binding lectin (MBL) using ELISA techniques.

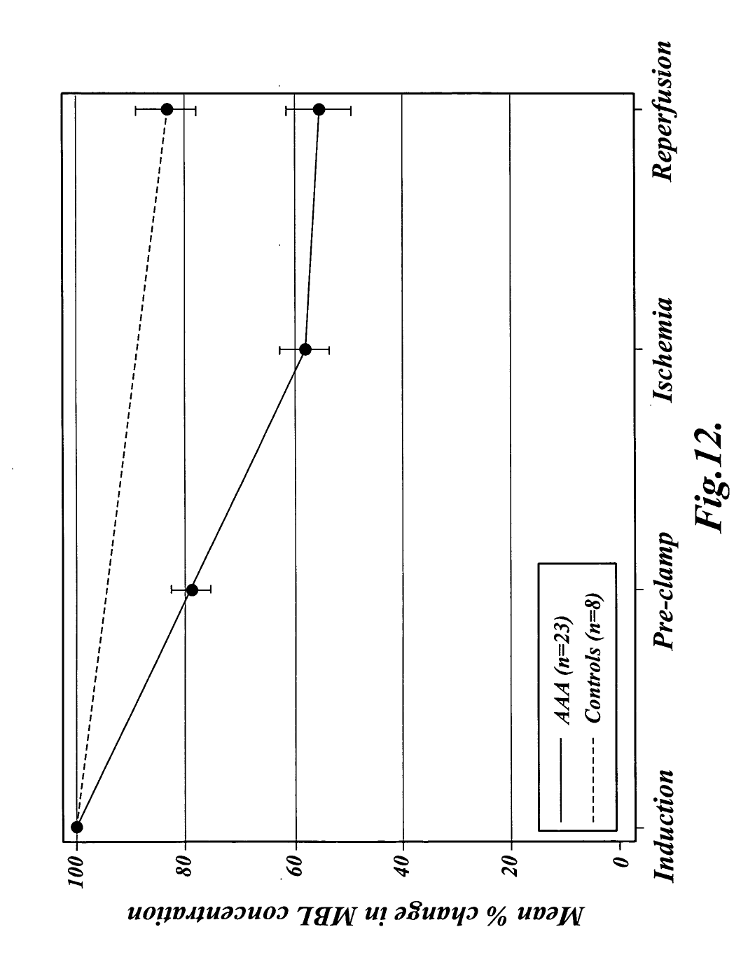

Results: The results of this study are shown in FIG. 12 which presents a graph showing the mean percentage change in MBL levels (y axis) at each of the various time points (x axis). Starting values for MBL are 100%, with relative decreases shown thereafter. As shown in FIG. 12, AAA patients (n=23) show a significant decrease in plasma MBL levels, averaging an approximate 41% decrease at time of ischemia/reperfusion following AAA. In contrast, in control patients (n=8) undergoing major abdominal surgery only a minor consumption of MBL was observed in the plasma samples.

The data presented provides a strong indication that the lectin-dependent pathway of the complement activation system is activated in the ischemia/reperfusion phase following AAA repair. The decrease in MBL levels appears to be associated with ischemia-reperfusion injury because the MBL levels drop significantly and rapidly when the clamped major vessel is reperfused after the end of the operation. In contrast, control sera of patients undergoing major abdominal surgery without a major ischemia-reperfusion insult only show a slight decrease in MBL plasma levels. In view of the well established contribution of complement activation in reperfusion injury, we conclude that activation of the lectin-dependent pathway on ischemic endothelial cells is a major factor in the pathology of reperfusion injury. Therefore, a specific transient blockade or reduction in the MASP-2-dependent lectin pathway of complement activation would be expected to have a significant beneficial therapeutic impact to improve the outcome of clinical procedures and diseases that involve a transient ischemic insult, e.g., myocardial infarction, gut infarction, burns, transplantation and stroke.

### Example 18

This example describes the use of the murine MAp19−/− strain expressing human MAp19 as an animal model for testing MAp19 inhibitory agents useful to treat Rheumatoid Arthritis.

Background and Rationale: Murine Arthritis Model: K/B×N T cell receptor (TCR) transgenic (tg) mice, a recently developed model of inflammatory arthritis (Kouskoff, V., et al., Cell 87:811-822, 1996; Korganow, A. S., et al., Immunity 10:451-461, 1999; Matsumoto, I., et al., Science 286:1732-1735, 1999; Maccioni, M., et al., J. Exp. Med. 195(8):1071-1077, 2002). The K/B×N mice spontaneously develop an autoimmune disease with most of the clinical, histological and immunological features of RA in humans (Ji, H., et al., Immunity 16:157-168, 2002). The murine disorder is joint specific, but is initiated then perpetuated by T, then B cell autoreactivity to glucose-6-phosphate isomerase (“GPI”), a ubiquitously expressed antigen. Further, transfer of serum (or purified anti-GPI Igs) from arthritic K/B×N mice into healthy animals provokes arthritis within several days. It has also been shown that polyclonal anti-GPI antibodies or a pool of monoclonal anti-GPI antibodies of the IgG1 isotype induce arthritis when injected into healthy recipients (Maccioni et al., 2002, supra). The murine model is relevant to human RA, because serum from RA patients has also been found to contain anti-GPI antibodies, which is not found in normal individuals. A C5-deficient mouse was tested in this system and found to block the development of arthritis (Ji, H., et al. Immunity 16:157-168, 2002). There was also strong inhibition of arthritis in C3 null mice, implicating the alternative pathway, however, MBP-A null mice did develop arthritis. In mice however, the presence of MBP-C may compensate for the loss of MBP-A.

Based on the observations described herein that MAp19 plays an essential role in the initiation of the lectin complement pathway, the K/B×N arthritic model is useful to screen for MAp19 inhibitory agents that are effective for use as a therapeutic agents to treat RA.

Methods: Serum from arthritic K/B×N mice is obtained at 60 days of age, pooled and injected (150-200 μl i.p.) into murine MAp19−/− recipients (obtained as described in Example 1), or murine MAp19−/− human MAp19+; and control littermates with or without MAp19 inhibitory agents (MoAb, inhibitory peptides and the like as described herein) at days 0 and 2. A group of normal mice are also pretreated with a MAp19 inhibitory agent for two days prior to receiving the injection of serum. A further group of mice receive an injection of serum at day 0, followed by a MAp19 inhibitory agent at day 6. A clinical index is evaluated over time with one point scored for each affected paw, ½ point scored for a paw with only mild swelling. Ankle thickness is also measured by a caliper (thickness is defined as the difference from day 0 measurement).

### Example 19

This example describes an assay for inhibition of complement-mediated tissue damage in an ex vivo model of rabbit hearts perfused with human plasma.

Background and Rationale: Activation of the complement system contributes to hyperacute rejection of xenografts. Previous studies have shown that hyperacute rejection can occur in the absence of anti-donor antibodies via activation of the alternative pathway (Johnston, P. S., et al., Transplant Proc. 23:877-879, 1991).

Methods: To determine whether isolated MAp19 inhibitory agents such as anti-MAp19 antibodies obtained as described in Example 9 are able to inhibit complement pathway in tissue damage, the anti-MAp19 MoAbs and antibody fragments may be tested using an ex vivo model in which isolated rabbit hearts are perfused with diluted human plasma. This model was previously shown to cause damage to the rabbit myocardium due to the activation of the alternative complement pathway (Gralinski, M. R., et al., Immunopharmacology 34:79-88, 1996).

### Example 20

This example describes an assay that measures neutrophil activation which is useful as a measure of an effective dose of a MAp19 inhibitory agent for the treatment of conditions associated with the lectin-dependent pathway in accordance with the methods of the invention.

Methods: A method for measuring neutrophil elastase has been described in Gupta-Bansal, R., et al., Mol. Immunol., 37:191-201, 2000. Briefly, the complex of elastase and serum α1-antitrypsin is measured with a two-site sandwich assay that utilizes antibodies against both elastase and α1-antitrypsin. Polystyrene microtiter plates are coated with a 1:500 dilution of anti-human elastase antibody (The Binding Site, Birmingham, UK) in PBS overnight at 4° C. After aspirating the antibody solution, wells are blocked with PBS containing 0.4% HAS for 2 h at room temperature. Aliquots (100 μl) of plasma samples that are treated with or without a MAp19 inhibitory agent are added to the wells. Following a 2 h incubation at room temperature, the wells are extensively rinsed with PBS. Bound elastase-α1-antitrypsin complex is detected by the addition of a 1:500 dilution of peroxidase conjugated-α1-antitrypsin antibody in blocking solution that is allowed to incubate for 1 h at room temperature. After washing the plate with PBS, 100 μl aliquots of TMB substrate are added. The reaction of TMB is quenched by the addition of 100 μl of phosphoric acid, and the plate is read at 450 nm in a microplate reader.

### Example 21

This example describes an animal model for testing MAp19 inhibitory agents useful to treat myocardial ischemia/reperfusion.

Methods: A myocardial ischemia-reperfusion model has been described by Vakeva et al., Circulation 97:2259-2267, 1998; and Jordan et al., Circulation 104(12):1413-1418, 2001. The described model may be modified for use in MAp19−/− MAp19+/+ mice as follows. Briefly, adult male mice are anesthetized. Jugular vein and trachea are cannulated and ventilation is maintained with 100% oxygen with a rodent ventilator adjusted to maintain exhaled CO₂ between 3.5% and 5%. A left thoracotomy is performed and a suture is placed 3 to 4 mm from the origin of the left coronary artery. Five minutes before ischemia, animals are given a MAp19 inhibitory agent, such as anti-MAp19 antibodies (e.g., in a dosage range of between 0.01 to 10 mg/kg). Ischemia is then initiated by tightening the suture around the coronary artery and maintained for 30 minutes, followed by four hours of reperfusion. Sham-operated animals are prepared identically without tightening the suture.

Analysis of Complement C3 Deposition: After reperfusion, samples for immunohistochemistry are obtained from the central region of the left ventricle, fixed and frozen at −80° C. until processed. Tissue sections are incubated with an HRP-conjugated goat anti-rat C3 antibody. Tissue sections are analyzed for the presence of C3 staining in the presence of anti-MAp19 inhibitory agents as compared with sham-operated control animals and MAp19−/− animals to identify MAp19 inhibitory agents that reduce C3 deposition in vivo.

### Example 22

This example describes the use of the MAp19−/− strain as an animal model for testing MAp19 inhibitory agents for the ability to protect transplanted tissue from ischemia/reperfusion injury.

Background/Rationale: It is known that ischemia/reperfusion injury occurs in a donor organ during transplantation. The extent of tissue damage is related to the length of ischemia and is mediated by complement, as demonstrated in various models of ischemia and through the use of complement inhibiting agents such as soluble receptor type 1 (CR1) (Weisman et al., Science 249:146-151, 1990; Mulligan et al., J. Immunol. 148:1479-1486, 1992; Pratt et al., Am. J. Path. 163(4):1457-1465, 2003). An animal model for transplantation has been described by Pratt et al., Am. J. Path. 163(4):1457-1465, which may be modified for use with the MAp19−/− mouse model and/or for use as a MAp19+/+ model system in which to screen MAp19 inhibitory agents for the ability to protect transplanted tissue from ischemia/reperfusion injury. The flushing of the donor kidney with perfusion fluid prior to transplantation provides an opportunity to introduce anti-MAp19 inhibitory agents into the donor kidney.

Methods: MAp19−/− and/or MAp19+/+ mice are anesthetized. The left donor kidney is dissected and the aorta is ligated cephalad and caudad to the renal artery. A portex tube catheter (Portex Ltd, Hythe, UK) is inserted between the ligatures and the kidney is perfused with 5 ml of Soltran Kidney Perfusion Solution (Baxter Health Care, UK) containing MAp19 inhibitory agents such as anti-MAp19 monoclonal antibodies (in a dosage range of from 0.01 mg/kg to 10 mg/kg) for a period of at least 5 minutes. Renal transplantation is then performed and the mice are monitored over time.

Analysis of Transplant Recipients: Kidney transplants are harvested at various time intervals and tissue sections are analyzed using anti-C3 to determine the extent of C3 deposition.

### Example 23

This example describes the use of a collagen-induced arthritis (CIA) animal model for testing MAp19 inhibitory agents useful to treat rheumatoid arthritis (RA).

Background and Rationale: Collagen-induced arthritis (CIA) represents an autoimmune polyarthritis inducible in susceptible strains of rodents and primates after immunization with native type II collagen and is recognized as a relevant model for human rheumatoid arthritis (RA) (see Courtney et al., Nature 283:666 (1980); Trenthan et al., J. Exp. Med. 146:857 (1977)). Both RA and CIA are characterized by joint inflammation, pannus formation and cartilage and bone erosion. The CIA susceptible murine strain DBA/1LacJ is a developed model of CIA in which mice develop clinically severe arthritis after immunization with Bovine type II collagen (Wang et al., J. Immunol. 164:4340-4347 (2000). A C5-deficient mouse strain was crossed with DBA/1LacJ and the resulting strain was found to be resistant to the development of CIA arthritis (Wang et al., 2000, supra).

Based on the observations described herein that MAp19 may play an essential role in the initiation of both the lectin and alternative pathways, the CIA arthritic model is useful to screen for MAp19 inhibitory agents that are effective for use as therapeutic agents to treat RA.

Methods: A MAp19−/− mouse is generated as described in Example 1. The MAp19−/− mouse is then crossed with a mouse derived from the DBA/1LacJ strain (The Jackson Laboratory). F1 and subsequent offspring are intercrossed to produce homozygous MAp19−/− in the DBA/1LacJ line.

Collagen immunization is carried out as described in Wang et al., 2000, supra. Briefly, wild-type DBA/1mice and MAp19−/− DBA/1LacJ mice are immunized with Bovine type II collagen (BCII) or mouse type II collagen (MCII) (obtained from Elastin Products, Owensville, Mo.), dissolved in 0.01 M acetic acid at a concentration of 4 mg/ml. Each mouse is injected intradermally at the base of the tail with 200 ug CII and 100 ug mycobacteria. Mice are re-immunized after 21 days and are examined daily for the appearance of arthritis. An arthritic index is evaluated over time with respect to the severity of arthritis in each affected paw.

MAp19 inhibitory agents are screened in the wild-type DBA/1LacJ CIA mice by injecting a MAp19 inhibitory agent such as anti-MAp19 monoclonal antibodies (in a dosage range of from 0.01 mg/kg to 10 mg/kg) at the time of collagen immunization, either systemically, or locally at one or more joints and an arthritic index is evaluated over time as described above. Anti-hMAp19 monoclonal antibodies as therapeutic agents can be easily evaluated in a MAp19−/−, hMAp19+/+ knock-in DBA/1LacJ CIA mouse model.

### Example 24

This example describes the use of a NZB/W F₁ animal model for testing MAp19 inhibitory agents useful to treat immune-complex mediated glomerulonephritis.

Background and Rationale: New Zealand black×New Zealand white (NZB/W) F₁ mice spontaneously develop an autoimmune syndrome with notable similarities to human immune-complex mediated glomerulonephritis. The NZB/W F₁ mice invariably succumb to glomerulonephritis by 12 months of age. As discussed above, it has been demonstrated that complement activation plays a significant role in the pathogenesis of immune-complex mediated glomerulonephritis. It has been further shown that the administration of an anti-C5 MoAb in the NZB/W F1 mouse model resulted in significant amelioration of the course of glomerulonepthritis (Wang et al., Proc. Natl. Acad. Sci. 93: 8563-8568 (1996)). Based on the observations described herein that MAp19 may play an essential role in the initiation of both the lectin and alternative pathways, the NZB/W F₁ animal model is useful to screen for MAp19 inhibitory agents that are effective for use as therapeutic agents to treat glomerulonephritis.

Methods: A MAp19−/− mouse is generated as described in Example 1. The MAp19−/− mouse is then seperately crossed with a mouse derived from the NZB and the NZW strains (The Jackson Laboratory). F1 and subsequent offspring are intercrossed to produce homozygous MAp19−/− in both the NZM and the NZB/W genetic backgrounds. To determine the role of MAp19 in the pathogenesis of glomerulonephritis in this model, the development of this disease in F1 animals resulting from crosses of either wild-type NZB×NZW mice or MAp19−/− NZB×MAp19−/−NZW mice are compared. At weekly intervals urine samples will be collected from the MAp19−/− and MAp19+/+ F1 mice and urine protein levels monitored for the presence of of anti-DNA antibodies (as described in Wang et al., 1996, supra). Histopathological analysis of the kidneys is also carried out to monitor the amount of mesangial matrix deposition.

The NZB/W F1 animal model will also be used to screen for MAp19 inhibitory agents that are effective for use as therapeutic agents to treat glomerulonephritis. At 18 weeks of age, wild-type NZB/W F1 mice are injected intraperitoneally with anti-MAp19 inhibitory agents, such as anti-MAp19 monoclonal antibodies (in a dosage range of from 0.01 mg/kg to 10 mg/kg) at a frequency of weekly or biweekly. The above-mentioned histopathological and biochemical markers of glomerulonephritis will be used to evaluate disease development in the mice treated with MAp19 inhibitory agents.

### Example 25

This example describes the use of a tubing loop as a model for testing MAp19 inhibitory agents useful to prevent tissue damage resulting from extracorporeal circulation (ECC) such as a cardiopulmonary bypass (CPB) circuit.

Background and Rationale: As discussed above, patients undergoing ECC during CPB suffer a systemic inflammatory reaction, which is partly caused by exposure of blood to the artificial surfaces of the extracorporeal circuit, but also by surface-independent factors like surgical trauma and ischemia-reperfusion injury (Butler, J., et al., Ann. Thorac. Surg. 55:552-9, 1993; Edmunds, L. H., Ann. Thorac. Surg. 66(Suppl):S112-6, 1998; Asimakopoulos, G., Perfusion 14:269-77, 1999). It has further been shown that the alternative complement pathway plays a predominant role in complement activation in CPB circuits, resulting from the interaction of blood with the artificial surfaces of the CPB circuits (see Kirklin et al., 1983, 1986, discussed supra). Therefore, based on the observations described herein that MAp19 may play an essential role in the initiation of both the lectin and alternative pathways, the tubing loop model is useful to screen for MAp19 inhibitory agents that are effective for use as therapeutic agents to prevent or treat an extracorporeal exposure-triggered inflammatory reaction.

Methods: A modification of a previously described tubing loop model for cardiopulmonary bypass circuits is utilized (see Gong et al., J. Clinical Immunol. 16(4):222-229 (1996)) as described in Gupta-Bansal et al., Molecular Immunology 37:191-201 (2000). Briefly, blood is freshly collected from a healthy subject in a 7 ml vacutainer tube (containing 7 units of heparin per ml of whole blood). Polyethylene tubing similar to what is used during CPB procedures (e.g., I.D. 2.92 mm; O.D. 3.73 mm, length: 45 cm) is filled with 1 ml of blood and closed into a loop with a short piece of silicone tubing. A control tubing containing heparinized blood with 10 mM EDTA was included in the study as a background control. Sample and control tubings were rotated vertically in a water bath for 1 hour at 37° C. After incubation, the blood samples were transferred into 1.7 ml microfuge tubes containing EDTA, resulting in a final concentration of 20 mM EDTA. The samples were centrifuged and the plasma was collected. MAp19 inhibitory agents, such as anti-MAp19 antibodies are added to the heparinized blood immediately before rotation. The plasma samples are then subjected to assays to measure the concentration C3a and soluble C5b-9 as described in Gupta-Bansal et al., 2000, supra.

### Example 26

This example describes the use of a rodent caecal ligation and puncture (CLP) model system for testing MAp19 inhibitory agents useful to treat sepsis or a condition resulting from sepsis, including severe sepsis, septic shock, acute respiratory distress syndrome resulting from sepsis and systemic inflammatory response syndrome.

Background and Rationale: As discussed above, complement activation has been shown in numerous studies to have a major role in the pathogenesis of sepsis (see Bone, R. C., Annals. Internal. Med. 115:457-469, 1991). The CLP rodent model is a recognized model that mimics the clinical course of sepsis in humans and is considered to be a reasonable surrogate model for sepsis in humans (see Ward, P., Nature Review Immunology 4:133-142 (2004). A recent study has shown that treatment of CLP animals with anti-C5a antibodies resulted in reduced bacteremia and greatly improved survival Huber-Lang et al., J. Immunol. 169:3223-3231 (2002). Therefore, based on the observations described herein that MAp19 may play an essential role in the initiation of both the lectin and alternative pathways, the CLP rodent model is useful to screen for MAp19 inhibitory agents that are effective for use as therapeutic agents to prevent or treat sepsis or a condition resulting from sepsis.

Methods: The CLP model is adapted from the model described in Huber-Lang et al., 2004, supra as follows. MAp19−/− and MAp19+/+ animals are anesthetized. A 2 cm midline abdominal incision is made and the cecum is tightly ligated below the ileocecal valve, avoiding bowel obstruction. The cecum is then punctured through and through with a 21-gauge needle. The abdominal incision was then closed in layers with silk suture and skin clips (Ethicon, Summerville, N.J.). Immediately after CLP, animals receive an injection of a MAp19 inhibitory agent such as anti-MAp19 monoclonal antibodies (in a dosage range of from 0.01 mg/kg to 10 mg/kg). Anti-hMAp19 monoclonal antibodies as therapeutic agents can be easily evaluated in a MAp19−/−, hMAp19+/+ knock-in CLP mouse model. The plasma of the mice are then analyzed for levels of complement-derived anaphylatoxins and respiratory burst using the assays described in Huber-Lang et al., 2004, supra.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of inhibiting lectin-dependent complement activation in a subject in need thereof, comprising administering to the subject an amount of a MAp19 inhibitory agent effective to inhibit lectin-dependent complement activation.

2. The method of claim 1 wherein the MAp19 inhibitory agent inhibits a protein complex comprising one or more MAp19 polypeptides.

3. The method of claim 1 wherein the MAp19 inhibitory agent specifically binds to a polypeptide comprising SEQ ID NO:3.

4. The method of claim 3 wherein the MAp19 inhibitory agent specifically binds to a polypeptide comprising SEQ ID NO:3 with an affinity of at least 10 times greater than it binds to other antigens in the complement system.

5. The method of claim 3 wherein the MAp19 inhibitory agent specifically binds to the polypeptide at a location within amino acid residues 160 to 170 of SEQ ID NO:3.

6. The method of claim 5 wherein the MAp19 inhibitory agent is selected from the group consisting of an antibody or fragment thereof, a polypeptide, a peptide, and a non-peptide agent.

7. The method of claim 6 wherein the MAp19 inhibitory agent is an antibody or fragment thereof that specifically binds to a portion of SEQ ID NO:3.

8. The method of claim 7 wherein the antibody or fragment thereof is monoclonal.

9. The method of claim 8 wherein the anti-MAp19 antibody or fragment thereof does not bind to MASP-2.

10. The method of claim 9 wherein the anti-MAp19 antibody is produced in a MAp19 deficient transgenic animal.

11. The method of claim 8 wherein the antibody is a chimeric, humanized or human antibody.

12. The method of claim 6 wherein the MAp19 inhibitory agent is a peptide derived from a polypeptide selected from the group consisting of human MASP-2, human MAp19, human MASP-1 that inhibits MAp19, human MBL that inhibits MAp19, human H-ficolin that inhibits MAp19, human M-ficolin, and human L-ficolin that inhibits MAp19.

13. The method of claim 6 wherein the MAp19 inhibitory agent is a non-peptide agent that specifically binds to a polypeptide comprising SEQ ID NO:3.

14. A method of inhibiting lectin-dependent complement activation in a subject in need thereof, comprising administering to the subject an amount of a MAp19 inhibitory agent effective to selectively inhibit lectin complement activation without substantially inhibiting C1q-dependent complement activation.

15. An anti-MAp19 antibody that does not bind to MASP-2.

16. The anti-MAp19 antibody of claim 15, wherein the antibody is monoclonal.

17. The anti-MAp19 antibody of claim 16 wherein the antibody inhibits lectin-dependent complement activation as measured in an in vitro assay.

18. A method of producing an anti-human MAp19 antibody that does not cross-react with human MASP-2 comprising generating a murine MAp19 deficient transgenic animal, integrating a human MASP-2 transgene into said murine MAp19 deficient animal, introducing a human MAp19 antigen into said animal, and selecting anti-human MAp19 antibodies that do not bind to human MASP-2.

19. The method of claim 18, further comprising the step of producing a monoclonal antibody.

20. An antibody obtained by the method of claim 18 or 19.

21. A composition for inhibiting lectin-dependent complement activation comprising a therapeutically effective amount of a MAp19 inhibitory agent and a pharmaceutically acceptable carrier.

22. A method of manufacturing a medicament for use in inhibiting the effects of lectin-dependent complement activation in living subjects in need thereof, comprising combining a therapeutically effective amount of a MAp19 inhibitory agent in a pharmaceutical carrier.

23. A method for treating a patient deficient in MAp19 by administering to the patient a polypeptide comprising SEQ ID NO:3, or a portion thereof.

24. A method of treating a subject suffering from a lectin-dependent complement mediated vascular condition comprising administering an amount of a MAp19 inhibitory agent effective to inhibit lectin-dependent complement activation.

25. The method of claim 24 wherein the vascular condition is selected from the group consisting of a cardiovascular condition, a cerebrovascular condition, a peripheral (e.g., musculoskeletal) vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy and percutaneous transluminal coronary angioplasty (PTCA).

26. A method of treating a subject suffering from a lectin-dependent complement mediated condition associated with an ischemia-reperfusion injury comprising administering an amount of a MAp19 inhibitory agent effective to inhibit lectin-dependent complement activation.

27. The method of claim 26 wherein the ischemia-reperfusion injury is associated with aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures.

28. A method of treating and/or preventing atherosclerosis in a subject in need thereof, comprising administering an amount of a MAp19 inhibitory agent effective to inhibit lectin-dependent complement activation.

29. A method of treating a subject suffering from a lectin-dependent complement mediated condition associated with an inflammatory gastrointestinal disorder comprising administering an amount of a MAp19 inhibitory agent effective to inhibit lectin-dependent complement activation.

30. The method of claim 29 wherein the inflammatory gastrointestinal disorder is selected from the group consisting of pancreatitis, Crohn's disease, ulcerative colitis, irritable bowel syndrome and diverticulitis.

31. A method of treating a subject suffering from a lectin-dependent complement mediated pulmonary condition comprising administering an amount of a MAp19 inhibitory agent effective to inhibit lectin-dependent complement activation.

32. The method of claim 31 wherein the pulmonary condition is selected from the group consisting of acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression and emphysema.

33. A method of inhibiting lectin-dependent complement activation in a subject that has undergone, is undergoing, or will undergo an extracorporeal reperfusion procedure comprising administering an amount of a MAp19 inhibitory agent effective to inhibit lectin-dependent complement activation.

34. The method of claim 33 wherein the extracorporeal reperfusion procedure is selected from the group consisting of hemodialysis, plasmapheresis, leukopheresis, extracorporeal membrane oxygenator (ECMO), heparin-induced extracorporeal membrane oxygenation LDL precipitation (HELP) and cardiopulmonary bypass (CPB).

35. A method of treating a subject suffering from a lectin-dependent complement mediated musculoskeletal condition comprising administering an amount of a MAp19 inhibitory agent effective to inhibit lectin-dependent complement activation.

36. The method of claim 35 wherein the musculoskeletal condition is selected from the group consisting of osteoarthritis, rheumatoid arthritis, gout, neuropathic arthropathy, psoriatic arthritis, juvenile rheumatoid arthritis, spondyloarthropathy, crystalline arthropathy and systemic lupus erythematosus (SLE).

37. A method of treating a subject suffering from a lectin-dependent complement mediated renal condition comprising administering an amount of a MAp19 inhibitory agent effective to inhibit lectin-dependent complement activation.

38. The method of claim 37 wherein the renal condition is selected from the group consisting of mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis and IgA nephropathy.

39. A method of treating a subject suffering from a lectin-dependent complement mediated skin condition comprising administering an amount of a MAp19 inhibitory agent effective to inhibit lectin-dependent complement activation.

40. The method of claim 39 wherein the skin condition is selected from the group consisting of psoriasis, autoimmune bullous dermatoses, eosinophilic spongiosis, bullous pemphigoid, epidermolysis bullosa acquisita, herpes gestationis, thermal burn injury and chemical burn injury.

41. A method of inhibiting lectin-dependent complement activation in a subject that has undergone, is undergoing, or will undergo an organ or tissue transplant procedure comprising administering an amount of a MAp19 inhibitory agent effective to inhibit lectin-dependent complement activation.

42. The method of claim 41 wherein the transplant procedure is selected from the group consisting of organ allotransplantation, organ xenotransplantation organ and tissue graft.

43. A method of treating a subject suffering from a lectin-dependent complement mediated condition associated with a nervous system disorder or injury comprising administering an amount of a MAp19 inhibitory agent effective to inhibit lectin-dependent complement activation.

44. The method of claim 43 wherein the nervous system disorder or injury is selected from the group consisting of multiple sclerosis, myasthenia gravis, Huntington's disease, amyotrophic lateral sclerosis, Guillain Barre syndrome, reperfusion following stroke, degenerative discs, cerebral trauma, Parkinson's disease, Alzheimer's disease, Miller-Fisher syndrome, cerebral trauma and/or hemorrhage, demyellination and meningitis.

45. A method of treating a subject suffering from a lectin-dependent complement mediated condition associated with a blood disorder comprising administering an amount of a MAp19 inhibitory agent effective to inhibit lectin-dependent complement activation.

46. The method of claim 45 wherein the blood disorder is selected from the group consisting of sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome.

47. A method of treating a subject suffering from a lectin-dependent complement mediated condition associated with a urogenital condition comprising administering an amount of a MAp19 inhibitory agent effective to inhibit lectin-dependent complement activation.

48. The method of claim 47 wherein the urogenital condition is selected from the group consisting of painful bladder disease, sensory bladder disease, chronic abacterial cystitis, interstitial cystitis, infertility, placental dysfunction and miscarriage and pre-eclampsia.

49. A method of treating a subject suffering from a lectin-dependent complement mediated condition associated with nonobese diabetes (Type-1 diabetes or Insulin-dependent diabetes mellitus) and/or complications associated with Type-1 or Type-2 (adult onset) diabetes comprising administering an amount of a MAp19 inhibitory agent effective to inhibit lectin-dependent complement activation.

50. The method of claim 49 wherein the complication associated with Type 1 or Type 2 diabetes is selected from the group consisting of angiopathy, neuropathy and retinopathy.

51. A method of inhibiting lectin-dependent complement activation in a subject that has undergone, is undergoing, or will undergo chemotherapeutic treatment and/or radiation therapy comprising administering an amount of a MAp19 inhibitory agent effective to inhibit lectin-dependent complement activation.

52. A method of treating a subject suffering from a malignancy comprising administering an amount of a MAp19 inhibitory agent effective to inhibit lectin-dependent complement activation.

53. A method of treating a subject suffering from an endocrine disorder comprising administering an amount of a MAp19 inhibitory agent effective to inhibit lectin-dependent complement activation.

54. The method of claim 53 wherein the endocrine disorder is selected from the group consisting of Hashimoto's thyroiditis, stress, anxiety and hormonal disorders involving regulated release of prolactin, growth or other insulin-like growth factor and adrenocorticotropin from the pituitary.

55. A method of treating a subject suffering from a complement mediated ophthalmologic condition comprising administering an amount of a MAp19 inhibitory agent effective to inhibit lectin-dependent complement activation.

56. The method of claim 55 wherein the ophthalmologic condition is age-related macular degeneration.
